(19) United States
(12) Patent Application Publication
(10) Pub. No.: US 2023/0226182 A1
(43) Pub. Date: Jul. 20, 2023

(54) CHIMERIC ANTIGEN RECEPTOR SPECIFIC FOR HLA

(71) Applicants: UCL BUSINESS LTD, London (GB); QUELL THERAPEUTICS LIMITED, London (GB)

(72) Inventors: Hans STAUSS, London (GB); Emma MORRIS, London (GB); Jenny MCGOVERN, London (GB); Elisa PETRIS, London Greater London (GB); Frederick DEAR, London Greater London (GB)

(21) Appl. No.: 17/771,191

(22) PCT Filed: Oct. 23, 2020
(86) PCT No.: PCT/GB2020/052695

§ 371 Date: Apr. 22, 2022

(30) Foreign Application Priority Data
Oct. 23, 2019 (GB) 1915384.0

(51) Int. Cl. A61K 39/00; C07K 14/47; C07K 14/725; C07K 16/28

(52) U.S. Cl. CPC A61K 39/4631; C07K 14/4703; C07K 14/7051; C07K 16/2833; A61K 39/46434; A61K 39/4611; A61K 39/4621; A61K 2239/13; A61K 2239/21; C07K 2319/03; A61K 2239/22; C07K 2319/02

(57) ABSTRACT

A vector comprising a first polynucleotide encoding a FOXP3 polypeptide and a second polynucleotide encoding a chimeric antigen receptor (CAR), wherein the CAR comprises an antigen recognition domain which specifically binds to a human leukocyte antigen (HLA), wherein the first polynucleotide and the second polynucleotide are operably linked to the same promoter, and wherein the first polynucleotide is upstream of the second polynucleotide.

### REFERENCE TO SEQUENCE LISTING

The Sequence Listing submitted Dec. 16, 2022, as a text file named “[FILE NAME],” created on Dec. 15, 2022, and having a size of [FILE SIZE] bytes is hereby incorporated by reference pursuant to 37 C.F.R. § 1.52(e)(5).

### FIELD OF THE INVENTION

The present invention relates to a vector encoding FOXP3 and a HLA-specific chimeric antigen receptor (CAR). The present invention also relates to engineered regulatory T cells comprising said vector and therapeutics uses thereof.

### BACKGROUND TO THE INVENTION

Allogeneic transplantation of foreign organs or tissues has lifesaving potential, but can lead to serious complications. After solid organ transplantation, immune-mediated rejection mandates the use of prolonged global immunosuppression and limits the life span of transplanted allografts. (Perkey, E. and Maillard, I., 2018. Annual Review of Pathology: Mechanisms of Disease, 13, pp. 219-245).

For example, acute cellular rejection occurs in 15-25% of liver transplant recipients on Tacrolimus based immunosuppression regimens (Choudhary, N. S., et al., 2017. Journal of clinical and experimental hepatology, 7(4), pp. 358-366). Acute immune-mediated rejection can be as high as 30-40% for kidney transplants (Roberts, D. M., et al., 2012. Transplantation, 94(8), pp. 775-783.),

While acute transplant rejection usually responds well to treatment, chronic rejection can represent a difficult situation. For example, a significant proportion of liver transplant patients do not respond to increased immunosuppression. Chronic rejection often leads to retransplantation or death (Choudhary, N. S., et al., 2017. Journal of clinical and experimental hepatology, 7(4), pp. 358-366).

Antigens present in the transplanted organ but not in the patient are a major cause of immune-mediated rejection. In particular, human leukocyte antigens (HLAs) present in the transplanted organ but not in the patient are an important cause of transplant rejection. HLA-A, HLA-B, and HLA-DR are major transplantation antigens and recent clinical data indicates that HLA matching also affects the clinical outcomes of HSCT. Acute rejection is primarily the result of a T cell-mediated response, whilst chronic rejection may also be due to antibody-mediated responses (Choo, S. Y., 2007. Yonsei medical journal, 48(1), pp. 11-23).

HLA typing can be used to match patients and donors for transplants and to reduce the risk of transplant rejection. HLA matching has, for example, had a large clinical impact in kidney and bone marrow transplantation. However, in heart, liver and lung transplantation allocation is based primarily on medical urgency, availability of donors, and waiting time (Sheldon, S. and Poulton, K., 2006. In Transplantation immunology, pp. 157-174, Humana Press; and Choo, S. Y., 2007. Yonsei medical journal, 48(1), pp. 11-23).

Moreover, HLA matching by Sanger Sequencing-Based Typing (SBT), the current standard of care, has important limitations. SBT typically only sequences a subset of HLA gene regions, precluding identification of potentially functional differences outside of those regions. In addition, DNA sequences generated by SBT have phase ambiguity, which affects as many as 53% of samples tested, providing a large source of potential error. HLA genotype ambiguities often require significant additional testing to ensure accurate matching of patient and potential donor (Allen, E. S., et al., 2018. Human immunology, 79(12), pp. 848-854).

Consequently, there remains a need for improved methods to down-regulate immune responses in transplant patients, in particular when there are antigens (e.g. HLAs) present in the transplanted organ but not in the patient.

### SUMMARY OF THE INVENTION

Regulatory T cells (Tregs) are a type of T cell that modulate the activity of the immune system. Generally, Tregs are immunosuppressive, down-regulating immune responses to stimuli. In particular, Tregs suppress activation and proliferation of conventional T cells, some types of which are directly involved in immune responses (e.g. cytotoxic T cells).

The suppressive effect of Tregs can be directed towards specific targets by expression of chimeric antigen receptors (CARs) that recognise antigens expressed on the surface of target cells. The present invention uses Tregs comprising CARs directed to an HLA antigen (e.g. HLA-A2) to induce tolerance to a transplant in a subject, or to treat and/or prevent transplant rejection.

However, there is a risk that engineered Tregs may lose the ability to suppress an immune response over time, which will reduce the efficacy of any Treg immunotherapy and/or require multiple infusions of the engineered Treg. There is also a risk that the engineered Tregs (i.e. Tregs expressing the CAR) may acquire an effector phenotype over time. Moreover, there is the potential to generate an engineered T effector cell as a by-product of generating of engineered Tregs, if, for example, T effector cells are present in the starting cell population.

This is problematic as, contrary to the immunosuppressive effect of Tregs, engineered T effector cells may increase or encourage immune-mediated damage of the target cells.

It has surprisingly been found that exogenous FOXP3 expression in regulatory T cells (Tregs) (which already express endogenous FOXP3) may enhance their regulatory function.

It has also surprisingly been found that exogenous FOXP3 expression in Tregs (which already express endogenous FOXP3) may reduce the risk of a Treg acquiring an effector phenotype and reduces the risk of generating an engineered T effector cell during generation of engineered Tregs.

Moreover, the inventors have determined that a configuration in which the polynucleotide encoding FOXP3 precedes that encoding the CAR in the 5′ to 3′ direction ensures that CAR expression can only occur when FOXP3 has been expressed and that expression of CAR without FOXP3 does not occur. This is a particular advantage in the present context of an engineered Treg, as it greatly reduces the risk of an engineered Treg acquiring an effector phenotype and/or reduces the risk associated with introducing the CAR into a T effector cell present in a starting population.

Accordingly, the present invention provides HLA-specific Tregs with enhanced efficacy and safety. The Tregs may be used for inducing tolerance to a transplant in a subject or for the treatment and/or prevention of transplant rejection.

In a first aspect, the present invention provides a vector comprising a first polynucleotide encoding a FOXP3 polypeptide and a second polynucleotide encoding a chimeric antigen receptor (CAR), wherein the CAR comprises an antigen recognition domain which specifically binds to a human leukocyte antigen (HLA), wherein the first polynucleotide and the second polynucleotide are operably linked to the same promoter, and wherein the first polynucleotide is upstream of the second polynucleotide.

Preferably, the antigen recognition domain specifically binds to HLA-A2.

The vector may comprise a polynucleotide encoding a cleavage site between the first polynucleotide and the second polynucleotide and/or an internal ribosome entry site (IRES) between the first polynucleotide and the second polynucleotide. Suitably, the vector may comprise a self-cleaving sequence between the first polynucleotide and the second polynucleotide, preferably wherein the self-cleaving sequence is a polynucleotide sequence encoding a 2A self-cleaving peptide. The 2A self-cleaving peptide may be selected from the group consisting of: P2A peptide, T2A peptide, E2A peptide, and F2A peptide.

The antigen recognition domain may be an antibody, an antibody fragment, or derived from an antibody. Suitably, the antigen recognition domain may be an antigen-binding fragment (Fab), a single chain antibody (scFv), or a single-domain antibody (sdAb). Preferably, the antigen recognition domain is a single chain antibody (scFv).

The CAR comprises a transmembrane (TM) domain and an intracellular signalling domain. The CAR may also comprise a hinge domain and/or one or more co-stimulatory domains. Preferably, the CAR comprises a CD8 hinge domain, a CD8 TM domain, a CD28 signalling domain, and a CD3 zeta signalling domain.

The vector may be a viral vector, preferably a retroviral vector or a lentiviral vector.

In another aspect the present invention provides an engineered T cell comprising a vector according to the present invention.

In another aspect the present invention provides an engineered regulatory T cell (Treg) comprising a vector according to the present invention.

In another aspect the present invention provides a polynucleotide encoding a FOXP3 polypeptide or a vector according to the present invention, for use in enhancing the ability of an engineered HLA-specific Treg to suppress an immune response, preferably an immune response against a cell expressing HLA.

The present invention also provides a method for enhancing the ability of an engineered HLA-specific Treg to suppress an immune response comprising introducing a polynucleotide encoding a FOXP3 polypeptide as described herein, or introducing a vector according to the present invention, into the Treg. Preferably the immune response is against a cell expressing the HLA.

The present invention also provides the use of a polynucleotide encoding a FOXP3 polypeptide as described herein, or the use of a vector as described herein, for enhancing the ability of an engineered HLA-specific Treg to suppress an immune response comprising introducing the polynucleotide encoding a FOXP3 polypeptide, or introducing the vector, into the Treg. Preferably the immune response is against a cell expressing the HLA.

In another related aspect the present invention provides a method for enhancing the ability of an engineered HLA-specific Treg to suppress an immune response comprising introducing a first polynucleotide encoding a FOXP3 polypeptide and a second polynucleotide encoding a HLA-specific CAR into the Treg.

In another related aspect the present invention provides a method for enhancing the ability of an engineered HLA-specific Treg to suppress an immune response comprising introducing a first polynucleotide encoding a FOXP3 polypeptide and a second polynucleotide encoding a HLA-specific CAR into a cell-containing sample, wherein:
(a) the cell-containing sample comprises or consists of Tregs; and/or
(b) the cell-containing sample comprises or consists of peripheral blood mononuclear cells (PBMCs) and Tregs are enriched from the cell-containing sample prior to or after introducing the first polynucleotide and/or second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs)) and Tregs are differentiated from the PSCs prior to or after introducing the first polynucleotide and/or the second polynucleotide.

Preferably the cell-containing sample has been isolated from the body.

In another aspect the present invention provides a polynucleotide encoding a FOXP3 polypeptide or a vector according to the present invention for use in reducing the risk of an engineered HLA-specific Treg acquiring an effector phenotype.

The present invention also provides a method for reducing the risk of an engineered HLA-specific Treg acquiring an effector phenotype comprising introducing a polynucleotide encoding a FOXP3 polypeptide, or introducing a vector according to the present invention, into the Treg.

The present invention also provides use of a polynucleotide encoding a FOXP3 polypeptide, or use of a vector as described herein, for reducing the risk of an engineered HLA-specific Treg acquiring an effector phenotype comprising introducing the polynucleotide encoding a FOXP3 polypeptide, or introducing the vector, into the Treg.

In another related aspect the present invention provides a method for reducing the risk of an engineered HLA-specific Treg acquiring an effector phenotype comprising introducing a first polynucleotide encoding a FOXP3 polypeptide and a second polynucleotide encoding a HLA-specific CAR into the Treg.

In another related aspect the present invention provides a method for reducing the risk of an engineered HLA-specific Treg acquiring an effector phenotype comprising introducing a first polynucleotide encoding a FOXP3 polypeptide and a second polynucleotide encoding a HLA-specific CAR into a cell-containing sample, wherein:
(a) the cell-containing sample comprises or consists of Tregs; and/or
(b) the cell-containing sample comprises or consists of PBMCs and Tregs are enriched from the cell-containing sample prior to or after introducing the first polynucleotide and/or the second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs)) and Tregs are differentiated from the PSCs prior to or after introducing the first polynucleotide and/or the second polynucleotide.

Preferably the cell-containing sample has been isolated from the body.

In another aspect the present invention provides a FOXP3 polypeptide or a vector according to the present invention for use in reducing the risk of generating an engineered HLA-specific T effector cell during generation of engineered HLA-specific Tregs.

The present invention also provides a method for reducing the risk of generating an engineered HLA-specific T effector cell during generation of engineered HLA-specific Tregs comprising introducing a polynucleotide encoding a FOXP3 polypeptide as described herein, or introducing a vector as described herein, into the Treg.

The present invention also provides use of a polynucleotide encoding a FOXP3 polypeptide as described herein, or use of a vector as described herein, for reducing the risk of generating an engineered HLA-specific T effector cell during generation of engineered HLA-specific Tregs comprising introducing the polynucleotide encoding the FOXP3 polypeptide, or introducing the vector, into the Treg.

In another related aspect the present invention provides a method for reducing the risk of generating an engineered HLA-specific T effector cell during generation of engineered HLA-specific Tregs, comprising introducing a first polynucleotide encoding a FOXP3 polypeptide and a second polynucleotide encoding a HLA-specific CAR into the Treg.

In another related aspect the present invention provides a method for reducing the risk of generating an engineered HLA-specific T effector cell during generation of engineered HLA-specific Tregs, comprising introducing a first polynucleotide encoding a FOXP3 polypeptide and a second polynucleotide encoding a HLA-specific CAR into a cell-containing sample, wherein:
(a) the cell-containing sample comprises Tregs and/or T effector cells; and/or
(b) the cell-containing sample comprises or consists of PBMCs and Tregs are enriched from the cell-containing sample prior to or after introducing the first polynucleotide and/or the second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably the cell-containing sample has been isolated from the body.

According to the present invention, the HLA referred to herein is preferably HLA-A2, e.g. preferably the HLA-specific CAR is a HLA-A2-specific CAR, the engineered HLA-specific Tregs are engineered HLA-A2-specific Tregs, and the engineered HLA-specific T effector cells are engineered HLA-A2-specific T effector cells.

In these aspects, the first polynucleotide and/or the second polynucleotide may be introduced by viral transduction, for example retroviral or lentiviral transduction. Preferably, the first polynucleotide and the second polynucleotide are introduced in a single vector, optionally wherein the first polynucleotide and the second polynucleotide are operably linked to the same promoter. More preferably, the single vector is a vector according to the present invention.

In another aspect the present invention provides an engineered Treg obtainable or obtained by a method according to the present invention.

In another aspect the present invention provides a pharmaceutical composition comprising a vector, an engineered T cell or an engineered Treg according to the present invention.

In another aspect the present invention provides a vector, an engineered T cell or Treg, or a pharmaceutical composition according to the present invention for use in induction of tolerance to a transplant in a subject, or for use in treatment and/or prevention of transplant rejection or graft-versus-host disease (GvHD) in a subject, or for use in treatment and/or prevention of an autoimmune or allergic disease in a subject, or for use in promoting tissue repair and/or tissue regeneration in a subject, or for use in ameliorating chronic inflammation in a subject. Preferably the subject is human.

In another related aspect the present invention provides a method of inducing tolerance to a transplant in a subject, or treating and/or preventing transplant rejection or GvHD in a subject, comprising administering a vector, an engineered T cell or Treg, or a pharmaceutical composition according to the present invention to the subject. Preferably the subject is human.

The method may comprise the following steps:
(i) isolation or provision of a cell-containing sample (e.g. from the subject), wherein the cell-containing sample comprises or consists of PBMCs; and
(ii) introducing a vector according to the invention into the cell-containing sample;
   wherein the cell-containing sample comprises or consists of Tregs; and/or wherein Tregs are enriched from the cell-containing sample prior to or after introducing the vector; and/or wherein Tregs are generated from the cell-containing sample prior to or after introducing the vector; and/or the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the vector.

### DESCRIPTION OF DRAWINGS

FIG. 1—the proliferation of Tconv cells (donor 1)

FIG. 1 shows the proliferation of Tconv cells transduced with a TCR construct, with and without peptide (shown with “*”), and the proliferation of the same cells in the presence of mock-transduced Tregs (white bars), Tregs transduced with a TCR construct (black bars) or Tregs transduced with a TCR construct and FOXP3 (grey bars) at different Treg:Tconv ratios.

FIG. 2—IL-2 production of Tconv cells (donor 1)

FIG. 2 shows the IL-2 production of Tconv cells transduced with a TCR construct, with and without peptide (shown with “*”), and the IL-2 production of the same cells in the presence of mock-transduced Tregs (white bars), Tregs transduced with a TCR construct (black bars) or Tregs transduced with a TCR construct and FOXP3 (grey bars) at different Treg:Tconv ratios.

FIG. 3—proliferation of Tconv cells (donor 2)

FIG. 3 shows the proliferation of Tconv cells (from a different donor to FIG. 1) transduced with a TCR construct, with and without peptide (shown with “*”), and the proliferation of the same cells in the presence of mock-transduced Tregs (white bars), Tregs transduced with a TCR construct (black bars) or Tregs transduced with a TCR construct and FOXP3 (grey bars) at different Treg:Tconv ratios.

FIG. 4—IL-2 production of Tconv cells (donor 2)

FIG. 4 shows the IL-2 production of Tconv cells (from a different donor to FIG. 2) transduced with a TCR construct with and without peptide (shown with “*”) and the proliferation of the same cells in the presence of mock-transduced Tregs (white bars), Tregs transduced with a TCR construct (black bars) or Tregs transduced with a TCR construct and FOXP3 (grey bars) at different Treg:Tconv ratios.

FIG. 5—mean fluorescence intensity (MFI) of Treg markers

FIG. 5 shows the mean fluorescence intensity (MFI) of Treg markers (FOXP3, CD25 and CTLA-4) in mock-transduced Treg or Treg transduced with TCR or TCR+FOXP3 analysed by flow cytometry at d7-10 after transduction. Dots are representative of individual experiments. 1-way ANOVA was used for statistical analysis p<0.05*, p<0.005**.

FIG. 6—MFI of FOXP3, CD25 and CTLA-4 in transduced Tregs

FIG. 6 shows the MFI of FOXP3, CD25 and CTLA-4 in transduced Tregs. Each line represents a single experiment showing MFI of markers on the same Treg transduced with TCR or TCR+FOXP3.

FIG. 7—proliferation of Tconv cells (donor 3)

FIG. 7 shows the proliferation of Tconv cells (from a different donor to FIGS. 1 and 3) transduced with a TCR construct, with and without peptide, and the proliferation of the same cells in the presence of mock-transduced Tregs (white bars), Tregs transduced with a TCR construct (black bars), Tregs transduced with a TCR construct and FOXP3 (grey bars) or Tconv cells transduced with a TCR construct and FOXP3, i.e. induced Tregs (red bars, right-hand bar of each data set), at different Treg:Tconv ratios.

FIG. 8—IL-2 production by Tconv cells (donor 3)

FIG. 8 shows the level of IL-2 production by Tconv cells (from the same donor as FIG. 7) transduced with a TCR construct, with and without peptide, and IL-2 production by the same cells in the presence of mock-transduced Tregs (white bars), Tregs transduced with a TCR construct (black bars), Tregs transduced with a TCR construct and FOXP3 (grey bars) or Tconv transduced with a TCR construct and FOXP3, i.e. induced Tregs (red bars right-hand bar of each data set), at different Treg:Tconv ratios.

FIG. 9—TCR transduced regulatory T cells can engraft into irradiated hosts but require exogenous FOXP3 expression to prevent accumulation of TCR+FOXP3− cells.

Thy1.1+CD4+CD25+Treg were isolated from lymph nodes and splenocytes of HLA-DRB*0401 transgenic mice by bead sort. Treg were transduced TCR, TCR+murine FOXP3 or cultured with virus-free supernatant (mock). 1 day after transduction TCR or TCR+FOXP3 transduced cells were injected into HLA-DRB*0401 transgenic hosts conditioned with 4Gy irradiation. 7 weeks later flow cytometry was used to determine the engraftment of transduced Treg A. Transduction efficiency was determined through expression of human variable 2.1 and murine Foxp3 on dl post-transduction B. Splenocytes from mice that received Treg transduced with TCR or TCR+FOXP3 were stained with Thy1.1 to identify transferred cells (top panel) and FOXP3 and TCR (bottom panel) C. Cumulative data showing fold change in transduction efficiency (left panel) and fold change in absolute number of transduced cells (right panel) relative to day of injection for Treg transduced with TCR or TCR+FOXP3 (n=3). Error bars show standard error of the mean. Statistical analysis by unpaired t test D. Representative expression of FOXP3 within transduced cells 7 weeks after transfer. Graphs show cumulative of percentage FOXP3+ cells within the transduced population at week 7 (left) and the fold change in FOXP3+ cells relative to the day of injection (n=3). Error bars show standard error of the mean. *p=>0.05, **p=>0.01 determined by unpaired t test.

FIG. 10—Treg expressing exogenous FOXP3 retain Treg functionality after 7 weeks in vivo whilst Tregs not expressing exogenous FOXP3 acquire the ability to produce effector cytokines

A Splenocytes were cultured for 4 hours with CD86+HLA-DR4+CHO cells pulsed with irrelevant peptide or 10 uM MBP. Production of IL-2 and IFNg was determined by flow cytometry. FACS plots show CD45.1 cells (top panel) containing Treg expressing TCR alone and Thy1.1 cells containing Treg expressing TCR+FOXP3. B Graphs show cumulative IL-2 and IFNg production by TCR-expressing (dark grey) and TCR+FOXP3-expressing (light grey) Treg. Error bars show standard deviation of the mean (n=3).

FIG. 11A—Illustrative HLA-A2-specific CAR construct designs

Schematic diagram of an illustrative vectors encoding a HLA-A2-specific CAR (denoted as A2 CAR): Construct F-C: illustrates a construct encoding 5′-FOXP3-P2A-A2 CAR-3′; Construct R-C: illustrates a construct encoding 5′-R-P2A-A2 CAR-3′, where R is another gene; Construct C: illustrates a construct encoding the A2 CAR only; Construct C-R: illustrates a construct encoding 5′-A2 CAR-P2A-R-3′, where R is another gene.

FIG. 11B—Generation of FOXP3/HLA-A2 CAR-Tregs

Schematic illustration showing the generation and expansion of the FOXP3/HLA-A2 CAR-Tregs. Phoenix-GP (P.gp) cells, a retroviral packaging line stably expressing gag pol, were seeded at 1×10⁶ cells/10 mm cell culture dish. The next day CD4+CD25hiCD127^{low} cells were isolated and activated with anti-CD3/CD28 beads in the absence of IL-2. On the same day P.gp cells were transfected with envelope and constructs encoding FOXP3/HLA.A2-CAR using Fugene transfection reagent. Two days after activation Tregs were transduced with g-retrovirus containing and supplemented with IL-2. Every 2 days cells were supplemented with additional media and IL-2. HLA.A2 dextramers were used to assess transduction efficacy at day 6. Tregs were further expanded with fresh anti-CD3/CD28 beads.

FIG. 12—HLA-A2-specific CAR and FOXP3 expression in transduced Tregs

FIG. 12 shows the HLA-A2-specific CAR (A2 CAR) expression levels, FOXP3 expression levels and the expression levels of another gene, R, in Tregs transduced with Construct F-C, Construct R-C, Construct C, and Construct C-R, compared to mock control Tregs, as determined by flow cytometry.

FIG. 12A shows CD3+CD4+ gated FACS plots for Dextramer (HLA) against SSC-A for the mock Tregs and for Tregs transduced with Construct F-C, Construct R-C, Construct C, and Construct C-R. Tregs transduced with each construct expressed the HLA-A2-specific CAR.

FIG. 12B shows CD3+CD4+Dextramer+ gated FACS plots for expression of FOXP3 against expression of another gene, R, for the mock Tregs and for Tregs transduced with Construct F-C, Construct R-C, Construct C, and Construct C-R. The gene, R, was expressed in high levels in Tregs transduced with both Construct R-C and Construct C-R. FOXP3 was expressed in all Tregs, but was noticeably higher in Construct F-C, particularly when compared to expression of the HLA-A2-specific CAR alone (Construct C).

FIG. 13—HLA-A2-specific CAR and FOXP3 expression in transduced and expanded Tregs

FIG. 13 shows the HLA-A2-specific CAR (A2 CAR) expression levels, FOXP3 expression levels and the expression levels of another gene, R, in expanded Tregs transduced with Construct F-C, Construct R-C, Construct C, and Construct C-R, compared to mock control Tregs, as determined by flow cytometry.

FIG. 13A shows CD3+CD4+ gated FACS plots for Dextramer (HLA) against SSC-A for the mock expanded Tregs and for Tregs transduced with Construct F-C, Construct R-C, Construct C, and Construct C-R and subsequently expanded. Tregs transduced with each construct still expressed the HLA-A2-specific CAR after expansion.

FIG. 13B shows CD3+CD4+Dextramer+ gated FACS plots for expression of FOXP3 against expression of another gene, R, for the mock expanded Tregs and for Tregs transduced with Construct F-C, Construct R-C, Construct C, and Construct C-R and subsequently expanded. FOXP3 expression decreased in Tregs transduced with Construct R-C, Construct C, and Construct C-R after expansion. In contrast, FOXP3 expression did not decrease in Tregs transduced Construct F-C after expansion. Consequently, FOXP3 expression was substantially higher in Construct F-C after expansion, particularly when compared to expression of the HLA-A2-specific CAR alone (Construct C).

FIG. 14—Phenotypic lineage of Tregs transduced with a construct encoding FOXP3 and an HLA-A2 specific CAR

FIG. 14 shows phenotypic lineage marker expression in Tregs transduced with Construct F-C, Construct R-C, Construct C, and Construct C-R.

Tregs transduced with construct F-C maintain Treg phenotypic lineage while enhancing FOXP3 expression.

### DETAILED DESCRIPTION

The suppressive properties of Tregs can be exploited therapeutically to improve and/or prevent immune-mediated organ damage in transplantation. The suppressive effect of Tregs can be directed towards specific targets by expression of chimeric antigen receptors (CARs) that recognise antigens expressed on the surface of target cells. In transplant rejection the CARs may be directed to an HLA antigen (e.g. HLA-A2) that is present in the graft (transplant) donor but not in the graft (transplant) recipient, whilst in GvHD the CARs may be directed to an HLA antigen (e.g. HLA-A2) that is present in the recipient but not in the graft (transplant) donor.

The present inventors have surprisingly found that exogenous FOXP3 expression in regulatory T cells (Tregs) (which already express endogenous FOXP3) may enhance their regulatory function.

The present inventors have surprisingly found that exogenous FOXP3 expression in Tregs (which already express endogenous FOXP3) may reduce the risk of a Treg acquiring an effector phenotype and reduces the risk of generating an engineered T effector cell during generation of engineered Tregs. In particular, a configuration in which FOXP3 precedes the CAR in the 5′ to 3′ direction ensures that CAR expression can only occur when FOXP3 has been expressed and that expression of CAR without FOXP3 does not occur.

Accordingly, the present invention provides HLA-specific Tregs (particularly HLA-A2-specific Tregs) with enhanced efficacy and safety. The Tregs may be used for the treatment and/or prevention of transplant rejection or graft-versus-host disease.

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Forkhead Box P3 Protein (FOXP3)

In the present invention, FOXP3 expression is increased in cells (e.g. Tregs) by introducing into the cells a polynucleotide (sometimes referred to herein as a first polynucleotide) encoding a FOXP3 polypeptide.

“FOXP3” is the abbreviated name of the forkhead box P3 protein. FOXP3 is a member of the FOX protein family of transcription factors and functions as a master regulator of the regulatory pathway in the development and function of regulatory T cells.

“Increasing FOXP3 expression” means to increase the levels of FOXP3 mRNA and/or protein in a cell (or population of cells) in comparison to a corresponding cell which has not been modified (or population of cells). For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a Treg or the population of cells is a population of Tregs.

Suitably, the level of FOXP3 mRNA and/or protein in a cell modified by according to the present invention (or a population of such cells) may be increased to at least 1.5-fold greater, 2-fold greater, or 5-fold greater than the level in a corresponding cell which has not been modified according to the present invention (or population of such cells). Preferably the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e. a polypeptide able to bind FOXP3 target DNA and function as a transcription factor regulating development and function of Tregs. Particularly, a FOXP3 polypeptide may have the same or similar activity to wildtype FOXP3 (SEQ ID NO.1), e.g. may have at least 40, 50, 60, 70, 80, 90, 95, 100, 110, 120, 130, 140 or 150% of the activity of the wildtype FOXP3 polypeptide. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Siegler et al. Annu. Rev. Immunol. 2006, 24: 209-26, incorporated herein by reference).

A “functional fragment of FOXP3” may refer to a portion or region of a FOXP3 polypeptide or a polynucleotide encoding a FOXP3 polypeptide that has the same of similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1(6), pp. 665-675; Lopes, J. E., et al., 2006. The Journal of Immunology, 177(5), pp. 3133-3142; and Lozano, T., et al, 2013. Frontiers in oncology, 3, p. 294.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide. FOXP3 variants may have the same or similar activity to a wildtype FOXP3 polypeptide or polynucleotide, e.g. may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions.

FOXP3 Polypeptide Sequences

Suitably, the FOXP3 polypeptide may comprise the polypeptide sequence of a human FOXP3, such as UniProtKB accession Q9BZS1 (SEQ ID NO: 1), or a functional fragment thereof:

FOXP3, UniProtKB accession Q9BZS1 (SEQ ID NO: 1):
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQQLVLEK
EKLSAMQAHLAGKMALTKASSVASSDKGSCCIVAAGSQGPVVPAWSGPRE
APDSLFAVRRHLWGSHGNSTFPEFLHNMDYFKFHNMRPPFTYATLIRWAI
LEAPEKQRTLNEIYHWFTRMFAFFRNHPATWKNAIRHNLSLHKCFVRVES
EKGAVWTVDELEFRKKRSQRPSRCSNPTPGP

In some embodiments of the invention, the FOXP3 polypeptide comprises an amino acid sequence which is at least 70% identical to SEQ ID NO: 1 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 1 or a functional fragment thereof. In some embodiments, the FOXP3 polypeptide comprises or consists of SEQ ID NO: 1 or a functional fragment thereof.

Suitably, the FOXP3 polypeptide may be a variant of SEQ ID NO: 1, for example a natural variant. Suitably, the FOXP3 polypeptide is an isoform of SEQ ID NO: 1. For example, the FOXP3 polypeptide may comprise a deletion of amino acid positions 72-106 relative to SEQ ID NO: 1. Alternatively, the FOXP3 polypeptide may comprise a deletion of amino acid positions 246-272 relative to SEQ ID NO: 1.

Suitably, the FOXP3 polypeptide comprises SEQ ID NO: 2 or a functional fragment thereof:

Illustrative FOXP3 polypeptide (SEQ ID NO: 2):
MPNPRPGKPSAPSLALGPSPGASPSWRAAPKASDLLGARGPGGTFQGRDL
RGGAHASSSSLNPMPPSQLQLPTLPLVMVAPSGARLGPLPHLQALLQDRP
HFMHQLSTVDAHARTPVLQVHPLESPAMISLTPPTTATGVFSLKARPGLP
PGINVASLEWVSREPALLCTFPNPSAPRKDSTLSAVPQSSYPLLANGVCK
WPGCEKVFEEPEDFLKHCQADHLLDEKGRAQCLLQREMVQSLEQVEELSA
MQAHLAGKMALTKASSVASSDKGSCCIVAAGSQGPVVPAWSGPREAPDSL
FAVRRHLWGSHGNSTFPEFLHNMDYFKFHNMRPPFTYATLIRWAILEAPE
KQRTLNEIYHWFTRMFAFFRNHPATWKNAIRHNLSLHKCFVRVESEKGAV
WTVDELEFRKKRSQRPSRCSNPTPGPEGRGSLLTCGDVEEN

Suitably the FOXP3 polypeptide comprises an amino acid sequence which is at least 70% identical to SEQ ID NO: 2 or a functional fragment thereof. Suitably, the FOXP3 polypeptide comprises an amino acid sequence which is at least 80%, at least 85%, at least 90%, at least 95%, at least 98% or at least 99% identical to SEQ ID NO: 2 or a functional fragment thereof. In some embodiments, the FOXP3 polypeptide comprises or consists of SEQ ID NO: 2 or a functional fragment thereof.

Suitably, the FOXP3 polypeptide may be a variant of SEQ ID NO: 2, for example a natural variant. Suitably, the FOXP3 polypeptide is an isoform of SEQ ID NO: 2 or a functional fragment thereof. For example, the FOXP3 polypeptide may comprise a deletion of amino acid positions 72-106 relative to SEQ ID NO: 2. Alternatively, the FOXP3 polypeptide may comprise a deletion of amino acid positions 246-272 relative to SEQ ID NO: 2.

FOXP3 Polynucleotide Sequences

Suitably, the polynucleotide encoding a FOXP3 polypeptide comprises or consists of a polynucleotide sequence set forth in SEQ ID NO: 3:

Illustrative FOXP3 polynucleotide (SEQ ID NO: 3):
ATGCCCAACCCCAGGCCTGGCAAGCCCTCGGCCCCTTCCTTGGCCCTTGG
CCCATCCCCAGGAGCCTCGCCCAGCTGGAGGGCTGCACCCAAAGCCTCAG
ACCTGCTGGGGGCCCGGGGCCCAGGGGGAACCTTCCAGGGCCGAGATCTT
CGAGGCGGGGCCCATGCCTCCTCTTCTTCCTTGAACCCCATGCCACCATC
GCAGCTGCAGCTGCCCACACTGCCCCTAGTCATGGTGGCACCCTCCGGGG
CACGGCTGGGCCCCTTGCCCCACTTACAGGCACTCCTCCAGGACAGGCCA
CATTTCATGCACCAGCTCTCAACGGTGGATGCCCACGCCCGGACCCCTGT
GCTGCAGGTGCACCCCCTGGAGAGCCCAGCCATGATCAGCCTCACACCAC
CCACCACCGCCACTGGGGTCTTCTCCCTCAAGGCCCGGCCTGGCCTCCCA
CCTGGGATCAACGTGGCCAGCCTGGAATGGGTGTCCAGGGAGCCGGCACT
GCTCTGCACCTTCCCAAATCCCAGTGCACCCAGGAAGGACAGCACCCTTT
CGGCTGTGCCCCAGAGCTCCTACCCACTGCTGGCAAATGGTGTCTGCAAG
TGGCCCGGATGTGAGAAGGTCTTCGAAGAGCCAGAGGACTTCCTCAAGCA
CTGCCAGGCGGACCATCTTCTGGATGAGAAGGGCAGGGCACAATGTCTCC
TCCAGAGAGAGATGGTACAGTCTCTGGAGCAGCAGCTGGTGCTGGAGAAG
GAGAAGCTGAGTGCCATGCAGGCCCACCTGGCTGGGAAAATGGCACTGAC
CAAGGCTTCATCTGTGGCATCATCCGACAAGGGCTCCTGCTGCATCGTAG
CTGCTGGCAGCCAAGGCCCTGTCGTCCCAGCCTGGTCTGGCCCCCGGGAG
GCCCCTGACAGCCTGTTTGCTGTCCGGAGGCACCTGTGGGGTAGCCATGG
AAACAGCACATTCCCAGAGTTCCTCCACAACATGGACTACTTCAAGTTCC
ACAACATGCGACCCCCTTTCACCTACGCCACGCTCATCCGCTGGGCCATC
CTGGAGGCTCCAGAGAAGCAGCGGACACTCAATGAGATCTACCACTGGTT
CACACGCATGTTTGCCTTCTTCAGAAACCATCCTGCCACCTGGAAGAACG
CCATCCGCCACAACCTGAGTCTGCACAAGTGCTTTGTGCGGGTGGAGAGC
GAGAAGGGGGCTGTGTGGACCGTGGATGAGCTGGAGTTCCGCAAGAAACG
GAGCCAGAGGCCCAGCAGGTGTTCCAACCCTACACCTGGCCCCTGA

In some embodiments of the invention, the polynucleotide encoding the FOXP3 polypeptide or variant comprises a polynucleotide sequence which is at least 70% identical to SEQ ID NO: 3 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 3 or a functional fragment thereof. In some embodiments of the invention, the polynucleotide encoding the FOXP3 polypeptide or variant comprises or consists of SEQ ID NO: 3 or a functional fragment thereof.

Suitably, the polynucleotide encoding a FOXP3 polypeptide comprises or consists of a polynucleotide sequence set forth in SEQ ID NO: 4:

Illustrative FOXP3 polynucleotide (SEQ ID NO: 4):
GAATTCGTCGACATGCCCAACCCCAGACCCGGCAAGCCTTCTGCCCCTTC
TCTGGCCCTGGGACCATCTCCTGGCGCCTCCCCATCTTGGAGAGCCGCCC
CTAAAGCCAGCGATCTGCTGGGAGCTAGAGGCCCTGGCGGCACATTCCAG
GGCAGAGATCTGAGAGGCGGAGCCCACGCCTCTAGCAGCAGCCTGAATCC
CATGCCCCCTAGCCAGCTGCAGCTGCCTACACTGCCTCTCGTGATGGTGG
CCCCTAGCGGAGCTAGACTGGGCCCTCTGCCTCATCTGCAGGCTCTGCTG
CAGGACCGGCCCCACTTTATGCACCAGCTGAGCACCGTGGACGCCCACGC
CAGAACACCTGTGCTGCAGGTGCACCCCCTGGAAAGCCCTGCCATGATCA
GCCTGACCCCTCCAACCACAGCCACCGGCGTGTTCAGCCTGAAGGCCAGA
CCTGGACTGCCCCCTGGCATCAATGTGGCCAGCCTGGAATGGGTGTCCCG
CGAACCTGCCCTGCTGTGCACCTTCCCCAATCCTAGCGCCCCCAGAAAGG
ACAGCACACTGTCTGCCGTGCCCCAGAGCAGCTATCCCCTGCTGGCTAAC
GGCGTGTGCAAGTGGCCTGGCTGCGAGAAGGTGTTCGAGGAACCCGAGGA
CTTCCTGAAGCACTGCCAGGCCGACCATCTGCTGGACGAGAAAGGCAGAG
CCCAGTGCCTGCTGCAGCGCGAGATGGTGCAGTCCCTGGAACAGCAGCTG
GTGCTGGAAAAAGAAAAGCTGAGCGCCATGCAGGCCCACCTGGCCGGAAA
GATGGCCCTGACAAAAGCCAGCAGCGTGGCCAGCTCCGACAAGGGCAGCT
GTTGTATCGTGGCCGCTGGCAGCCAGGGACCTGTGGTGCCTGCTTGGAGC
GGACCTAGAGAGGCCCCCGATAGCCTGTTTGCCGTGCGGAGACACCTGTG
GGGCAGCCACGGCAACTCTACCTTCCCCGAGTTCCTGCACAACATGGACT
ACTTCAAGTTCCACAACATGAGGCCCCCCTTCACCTACGCCACCCTGATC
AGATGGGCCATTCTGGAAGCCCCCGAGAAGCAGCGGACCCTGAACGAGAT
CTACCACTGGTTTACCCGGATGTTCGCCTTCTTCCGGAACCACCCCGCCA
CCTGGAAGAACGCCATCCGGCACAATCTGAGCCTGCACAAGTGCTTCGTG
CGGGTGGAAAGCGAGAAGGGCGCCGTGTGGACAGTGGACGAGCTGGAATT
TCGGAAGAAGCGGTCCCAGAGGCCCAGCCGGTGTAGCAATCCTACACCTG
GCCCTGAGGGCAGAGGAAGTCTGCTAACATGCGGTGACGTCGAGGAGAAT
CC

In some embodiments of the invention, the polynucleotide encoding the FOXP3 polypeptide or variant comprises a polynucleotide sequence which is at least 70% identical to SEQ ID NO: 4 or a functional fragment thereof. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO: 4 or a functional fragment thereof. In some embodiments of the invention, the polynucleotide encoding the FOXP3 polypeptide or variant comprises or consists of SEQ ID NO: 4 or a functional fragment thereof.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

HLA Specific Chimeric Antigen Receptor

In the present invention, HLA-specific cells are generated by introducing into the cells a polynucleotide (sometimes referred to herein as a second polynucleotide) encoding a HLA-specific chimeric antigen receptor (CAR).

“Chimeric antigen receptor” or “CAR” or “CARs” as used herein refers to engineered receptors which confer an antigen specificity onto cells, e.g. Tregs. CARs are also known as artificial T-cell receptors, chimeric T-cell receptors or chimeric immunoreceptors. The CARs of the invention comprise a binding domain specific for a HLA, preferably HLA-A2, optionally a hinge domain, a transmembrane domain, and an endodomain (comprising an intracellular signalling domain and optionally one or more co-stimulatory domains).

CAR-encoding polynucleotides may be transferred to cells using, for example, retroviral vectors. In this way, a large number of antigen-specific T cells can be generated for adoptive cell transfer. When the CAR binds the target-antigen, this results in the transmission of an activating signal to the cell (e.g. Treg) it is expressed on. Thus, the CAR may direct an engineered Treg towards cells expressing the targeted antigen, thereby supressing an immune response against the antigen or a cell comprising the antigen.

Antigen Recognition Domain

The CAR of the invention comprises an antigen recognition domain.

The “antigen recognition domain” as used herein refers to the extracellular part of the CAR that defines the antigen-binding capability of the CAR. In certain aspects of the invention, the antigen recognition domain provides the CAR with the ability to bind to a HLA. Thus, the antigen recognition domain targets an HLA.

The human leukocyte antigen (HLA) system or complex is a gene complex encoding the major histocompatibility complex (MHC) proteins in humans. HLAs are responsible for the regulation of the immune system in humans.

Suitably, the HLA is selected from the group consisting of: HLA-A2, HLA-A1, HLA-00701, HLA-A3, HLA-A11, and HLA-A2402. Preferably the HLA is HLA-A2.

“HLA-A2” may also be referred to as HLA-A*02, HLA-A02, and HLA-A*2. HLA-A*02 is one particular class I major histocompatibility complex (MHC) allele group at the HLA-A locus.

Suitably, the CAR may comprise an antigen binding domain which is capable of binding to a HLA (preferably HLA-A2), that is present in a graft (transplant) donor but not in a graft (transplant) recipient or vice versa. For example, where the transplant is an organ transplant, the HLA (preferably HLA-A2) may be present in the transplanted organ but not in the patient. Where the transplant is a HSCT (e.g. a bone marrow transplant), the HLA (preferably HLA-A2) may be present in the patient but not in the transplant.

The antigen recognition domain may bind, suitably specifically bind, one or more region or epitope within a HLA (preferably HLA-A2). An epitope, also known as antigenic determinant, is the part of an antigen that is recognised by an antigen recognition domain (e.g. an antibody). In other words, the epitope is the specific piece of the antigen to which an antibody binds.

Suitably, the antigen recognition domain binds, suitably specifically binds, to one region or epitope within a HLA (preferably HLA-A2). It will be appreciated by a skilled person that specific binding may occur at more than one region within the HLA (preferably HLA-A2), e.g. due to protein/polypeptide folding.

The antigen recognition domain used in the present invention may selectively or specifically bind to a HLA (preferably HLA-A2), and thus may have a greater binding affinity for a HLA (preferably HLA-A2) as compared to its binding affinity for other proteins/molecules. Suitably, “specifically binds” as used herein means that the antigen recognition domain does not bind to other proteins or binds with a greatly reduced affinity compared to the binding to a HLA (preferably HLA-A2), to which it specifically binds (e.g. with an affinity of at least 10, 50, 100, 500, 1000 or 10000 times less than its affinity for the HLA (preferably HLA-A2)). Thus, the antigen recognition domain as referred to herein may bind to a HLA (preferably HLA-A2) with at least 10, 50, 100, 500, 1000 or 10000 times the affinity of its binding to other proteins. The binding affinity of the antigen recognition domain can be determined using methods well known in the art such, for example using the Lineweaver-Burk method, or by using commercially available binding model software, such as the 1:1 binding model in the BIAcore 1000 Evaluation software. Suitably, the HBS-P buffer system (0.01M Hepes, pH 7.4, 0.15M NaCl, 0.05% surfactant P20) is used.

The antigen recognition domain (also known as an antigen-specific targeting domain) may be any protein or peptide that possesses the ability to specifically recognise and bind to a HLA (preferably HLA-A2). The antigen recognition domain includes any naturally occurring, synthetic, semi-synthetic, or recombinantly produced binding partner for a HLA (preferably HLA-A2). Illustrative antigen recognition domains include antibodies, antibody fragments or derivatives, extracellular domains of receptors, ligands for cell surface molecules/receptors, or receptor binding domains thereof, and tumour binding proteins.

Preferably, the antigen recognition domain is, or is derived from, an antibody (Ab). An antibody-derived antigen recognition domain can be a fragment of an antibody or a genetically engineered product of one of more fragments of the antibody, which fragment is involved in binding with the antigen. Examples include a camelid antibody (VHH), an antigen-binding fragment (Fab), a variable region (Fv), a single chain antibody (scFv), a single-domain antibody (sdAb), a heavy chain variable region (VH), a light chain variable region (VL), and a complementarity determining region (CDR).

In preferred embodiments, the antigen recognition domain is a single chain antibody (scFv).

An antibody recognises an antigen via the fragment antigen-binding (Fab) variable region. Antibodies are glycoproteins belonging to the immunoglobulin superfamily. They constitute most of the gamma globulin fraction of the blood proteins. They are typically made of basic structural units, each with two large heavy chains and two small light chains. Camelid antibodies (VHH) lack light chains, and consist of two heavy chains attached to variable domains.

“Antigen-binding fragment” (Fab) refers to a region on an antibody that binds to antigens. It is composed of one constant and one variable region of each of the heavy and the light chain.

“Fv” refers to the smallest fragment of an antibody to bear the complete antigen binding site. An Fv fragment consists of the variable regions of a single light chain bound to the variable region of a single heavy chain.

“Single chain antibody” (scFv) refers to an engineered antibody consisting of a light chain variable region and a heavy chain variable region connected to one another directly or via a peptide linker sequence. Suitable linkers can be readily selected and can be of any suitable length, such as from 1 amino acid (e.g. Gly) to 30 amino acids, e.g. from any one of 2, 3, 4, 5, 6, 7, 8, 9, or 10 amino acids to any one of 12, 15, 18, 20, 21, 25, 30 amino acids, for example, 5-30, 5-25, 6-25, 10-15, 12-25, 15 to 25 etc. The peptide linker sequence is usually about 10 to 25 amino acids in length, rich in glycine for flexibility, and serine or threonine for solubility. Exemplary flexible linkers include glycine polymers (G)n, where n is an integer of at least one, glycine-serine polymers, glycine-alanine polymers, alanine-serine polymers, and other flexible linkers known in the art. The linker may comprise 1 or more “GS” domains. Linkers can have different characteristics and for example may be flexible, rigid or cleavable. The peptide linker sequence can either connect the N-terminus of the heavy chain variable region with the C-terminus of the light chain variable region, or vice versa. The heavy chain variable region and the light chain variable region may be linked via a linker sequence of (X)n, where X is any amino acid and n is an integer of between 1 and 30. The linker sequence may be any linker sequence known in the art.

“Single-domain antibody” (sdAb), also known as a nanobody, refers to an antibody fragment consisting of a single monomeric variable antibody domain. Accordingly, a sdAb may be a heavy chain variable region (VH) or a light chain variable region (VL).

“Heavy chain variable region” or “VH” refers to the fragment of the heavy chain of an antibody that contains three CDRs interposed between flanking stretches known as framework regions, which are more highly conserved than the CDRs and form a scaffold to support the CDRs.

“Light chain variable region” or “VL” refers to the fragment of the light chain of an antibody that contains three CDRs interposed between framework regions.

“Complementarity determining region” or “CDR” with regard to antibody or antigen-binding fragment thereof refers to a highly variable loop in the variable region of the heavy chain of the light chain of an antibody. CDRs can interact with the antigen conformation and largely determine binding to the antigen (although some framework regions are known to be involved in binding). The heavy chain variable region and the light chain variable region each contain 3 CDRs (heavy chain CDRs 1, 2 and 3 and light chain CDRs 1, 2 and 3, numbered from the amino to the carboxy terminus).

The CDRs of the variable regions of a heavy and light chain of an antibody can be predicted from the heavy and light chain variable region sequences of the antibody, using prediction software available in the art, e.g. using the Abysis algorithm, or using the IMGT/V-QUEST software, e.g. the IMGT algorithm (ImMunoGeneTics) which can be found at www.IMGT.org, (see for example Lefranc et al, 2009 NAR 37:D1006-D1012 and Lefranc 2003, Leukemia 17: 260-266). CDR regions identified by either algorithm are considered to be equally suitable for use in the invention. CDRs may vary in length, depending on the antibody from which they are predicted and between the heavy and light chains. Thus, the three heavy chain CDRs of an intact antibody may be of different lengths (or may be of the same length) and the three light chain CDRs of an intact antibody may be of different lengths (or may be of the same length). A CDR for example, may range from 2 or 3 amino acids in length to 5, 6, 7, 8, 9, 10, 11, 12, 13, 14, 15, 16, 17, 18, 19 or 20 amino acids in length. Particularly, a CDR may be from 3-14 amino acids in length, e.g. at least 3 amino acids and less than 15 amino acids.

It should be note that the Kabat nomenclature is followed herein where necessary, in order to define the positioning of the CDRs (Kabat et al, 1991, 5th Ed. Public Health Service, National Institutes of Health, Bethesda, Md., 647-669).

Antibodies, and derivatives and fragments thereof, that specifically bind to a HLA (preferably HLA-A2) can be prepared using methods well known by those of skill in the art. Such methods include phage display, methods to generate human or humanized antibodies, or methods using transgenic animals or plants engineered to produce human antibodies. Phage display libraries of partially or fully synthetic antibodies are available and can be screened for an antibody or fragment thereof that can bind to a HLA (preferably HLA-A2). Phage display libraries of human antibodies are also available. Once identified, the amino acid sequence or polynucleotide sequence encoding for the antibody (or derivative or fragment thereof) can be isolated and/or determined. The sequence of the antibody can be used to design suitable derivatives or fragments thereof.

Examples of antibodies, and fragments and derivatives thereof that can be used in the invention are further described below.

The antigen recognition domain may comprise at least one CDR (e.g. CDR3), which can be predicted from an antibody (or antibody fragment) which binds to a HLA (preferably HLA-A2) or a variant of such a predicted CDR (e.g. a variant with one, two or three amino acid substitutions). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13: 1882-91).

In this respect, the antigen recognition domain may comprise one or more variable heavy chain CDRs, e.g. one, two or three variable heavy chain CDRs. Alternatively, or additionally, the antigen recognition domain may comprise one or more variable light chain CDRs, e.g. one, two or three variable light chain CDRs. The antigen recognition domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to a HLA (preferably HLA-A2) or may be selected from one of the CDR sequences provided below.

The antigen recognition domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen recognition domain comprises three variable heavy chain CDRs (CDR1, CDR2 and CDR3) and/or three variable light chain CDRs (CDR1, CDR2 and CDR3).

The one or more CDRs present within the antigen recognition domain may not all be from the same antibody, as long as the domain has the binding activity described above. Thus, one CDR may be predicted from the heavy or light chains of an antibody which binds to a HLA (preferably HLA-A2) whilst another CDR present may be predicted from a different antibody which also binds to a HLA (preferably HLA-A2). In this instance, it may be preferred that CDR3 be predicted from an antibody that binds to a HLA (preferably HLA-A2). Particularly however, if more than one CDR is present in the antigen recognition domain, it is preferred that the CDRs are predicted from antibodies which bind to a HLA (preferably HLA-A2), particularly the same region or epitope of the HLA. A combination of CDRs may be used from different antibodies, particularly from antibodies that bind to the same region or epitope.

In a particularly preferred embodiment, the antigen recognition domain comprises three CDRs predicted from the variable heavy chain sequence of an antibody (or antibody fragment) which binds to a HLA (preferably HLA-A2) and/or three CDRs predicted from the variable light chain sequence of an antibody (or antibody fragment) which binds to a HLA (preferably HLA-A2) (preferably the same antibody or antibody fragment).

In some embodiments, the antigen recognition domain is, or is derived from an antibody (e.g. is a Fab, scFv, or sdAb) wherein the antibody comprises one or more CDR regions, selected from SEQ ID NOs: 5-133, or derivatives thereof (e.g. derivatives comprising 1, 2 or 3 substitutions, preferably one substitution). In other words, in some embodiments the antigen recognition domain comprises one or more CDR regions, selected from SEQ ID NOs: 5-133, or derivatives thereof (e.g. derivatives comprising 1, 2 or 3 substitutions, preferably one substitution). Suitably, the antigen recognition domain comprises three CDR regions selected from SEQ ID NOs: 5-133, or derivatives thereof.

| Name | CDR1 | CDR2 | CDR3 |
|---|---|---|---|
| VH CDRs 1 | DYGMH | FIRNDGSDKYYADSVKG | NGESGPLDYWYFDL |
| | (SEQ ID NO: 5) | (SEQ ID NO: 6) | (SEQ ID NO: 7) |
| VH CDRs 2 | DYGMH | FIRNDGSDKYYADSVKG | NGESGPLDYWYLDL |
| | (SEQ ID NO: 8) | (SEQ ID NO: 9) | (SEQ ID NO: 10) |
| VH CDRs 3 | DYGMH | FIRNDGSDKYYADSVRG | NGESGPLDYWYFDL |
| | (SEQ ID NO: 11) | (SEQ ID NO: 12) | (SEQ ID NO: 13) |
| VH CDRs 4 | DYGMH | FIRNDGSDKYYADSVKG | NGESGPLDYWYFDL |
| | (SEQ ID NO: 14) | (SEQ ID NO: 15) | (SEQ ID NO: 16) |
| VL CDRs 1 | QASQDISNYLN | DASNLET | QQYDNLPPT |
| | (SEQ ID NO: 17) | (SEQ ID NO: 18) | (SEQ ID NO: 19) |
| VL CDRs 2 | QSSLDISHYLN | DASNLET | QQYDNLPLT |
| | (SEQ ID NO: 20) | (SEQ ID NO: 21) | (SEQ ID NO: 22) |
| VL CDRs 3 | RASHGINNYLA | AASTLQS | QQYDSYPPT |
| | (SEQ ID NO: 23) | (SEQ ID NO: 24) | (SEQ ID NO: 25) |
| VL CDRs 4 | QASQDISNYLN | DASNLET | QQYSSFPLT |
| | (SEQ ID NO: 26) | (SEQ ID NO: 27) | (SEQ ID NO: 28) |
| VL CDRs 5 | QASQDISNYLN | DETHLDS | QQYDSLPPT |
| | (SEQ ID NO: 29) | (SEQ ID NO: 30) | (SEQ ID NO: 31) |
| VL CDRs 6 | QASQDISNYLN | DASNLET | QQYDNLPIT |
| | (SEQ ID NO: 32) | (SEQ ID NO: 33) | (SEQ ID NO: 34) |
| VL CDRs 7 | QASQDISNYLN | DASNLET | QQYDNLPST |
| | (SEQ ID NO: 35) | (SEQ ID NO: 36) | (SEQ ID NO: 37) |
| VL CDRs 8 | QASQDISNYLN | DASNLET | QQYNTYPLT |
| | (SEQ ID NO: 38) | (SEQ ID NO: 39) | (SEQ ID NO: 40) |
| VL CDRs 9 | QASQDISNYLN | DASNLET | QQYHTYPLT |
| | (SEQ ID NO: 41) | (SEQ ID NO: 42) | (SEQ ID NO: 43) |
| VL CDRs 10 | QASQDISNYLN | DASNLET | QQYDNLPLT |
| | (SEQ ID NO: 44) | (SEQ ID NO: 45) | (SEQ ID NO: 46) |
| VL CDRs 11 | RTSQGISSALA | DASSLES | QQFNNYPLT |
| | (SEQ ID NO: 47) | (SEQ ID NO: 48) | (SEQ ID NO: 49) |
| VL CDRs 12 | QASQDISNYLA | AASNLQS | LQDSSYPPT |
| | (SEQ ID NO: 50) | (SEQ ID NO: 51) | (SEQ ID NO: 52) |
| VL CDRs 13 | RASQSISSWLA | KASNLQS | QQYSNYPLT |
| | (SEQ ID NO: 53) | (SEQ ID NO: 54) | (SEQ ID NO: 55) |
| VL CDRs 14 | RASHGISNYFA | ATSTLOS | QQYSSYPLT |
| | (SEQ ID NO: 56) | (SEQ ID NO: 57) | (SEQ ID NO: 58) |
| VL CDRs 15 | RASRGISNYLA | ATSTLQS | QQYDSYPPT |
| | (SEQ ID NO: 59) | (SEQ ID NO: 60) | (SEQ ID NO: 61) |
| VH CDRs 5 | SYAIS | GIIPIFGTANYAQKFQG | GGYDSRGSYYYMDV |
| | (SEQ ID NO: 62) | (SEQ ID NO: 63) | (SEQ ID NO: 64) |
| VH CDRs 6 | SYAMH | VISYDGSNKYYADSVKG | DREELLALFGGMDV |
| | (SEQ ID NO: 65) | (SEQ ID NO: 66) | (SEQ ID NO: 67) |
| VH CDRs 7 | SYAIS | GIIPIFGTANYAQKFQG | PQSRWLQSGDAFDI |
| | (SEQ ID NO: 68) | (SEQ ID NO: 69) | (SEQ ID NO: 70) |
| VH CDRs 8 | SYAMH | RINAGNGNTKYSQKFQG | DLTGTLLFDY |
| | (SEQ ID NO: 71) | (SEQ ID NO: 72) | (SEQ ID NO: 73) |
| VH CDRs 9 | SYAIS | GIIPIFGTANYAQKFQG | RAERWLHLSGAFDI |
| | (SEQ ID NO: 74) | (SEQ ID NO: 75) | (SEQ ID NO: 76) |
| VH CDRs 10 | SYAMS | AISGSGGSTYYADSVKG | GHYGDYV |
| | (SEQ ID NO: 77) | (SEQ ID NO: 78) | (SEQ ID NO: 79) |
| VH CDRs 11 | SYAMH | WINAGNGNTKYSQKFQG | VSSGGAFDI |
| | (SEQ ID NO: 80) | (SEQ ID NO: 81) | (SEQ ID NO: 82) |
| VH CDRs 12 | SYGFS | EIIPMFGTANYAQKLQG | VPRSSSGYNYGMDV |
| | (SEQ ID NO: 83) | (SEQ ID NO: 84) | (SEQ ID NO: 85) |
| VH CDRs 13 | TNSGAWS | RTYYRSKWSTDYALSLQS | ENWNSGGFDY |
| | (SEQ ID NO: 86) | (SEQ ID NO: 87) | (SEQ ID NO: 88) |
| VH CDRs 14 | SYAIS | GIIPIFGTANYAQKFQG | AASRWEPGDAFDI |
| | (SEQ ID NO: 89) | (SEQ ID NO: 90) | (SEQ ID NO: 91) |
| VH CDRs 15 | SYDIS | WISAYNGNTNYAQKLQG | GGRWLRSASSFDY |
| | (SEQ ID NO: 92) | (SEQ ID NO: 93) | (SEQ ID NO: 94) |
| VH CDRs 16 | DHYMS | WISAYNGNTNYAQKLQG | GLDSSAYQGRAFDI |
| | (SEQ ID NO: 95) | (SEQ ID NO: 96) | (SEQ ID NO: 97) |
| VL CDRs 16 | SGSSSNIGGNAVN | SNNQRPS | TAWDDSLRGYL |
| | (SEQ ID NO: 98) | (SEQ ID NO: 99) | (SEQ ID NO: 100) |
| VL CDRs 17 | GGNNIGSKSVH | DDSDRPS | HVWDAKTNHQV |
| | (SEQ ID NO: 101) | (SEQ ID NO: 102) | (SEQ ID NO: 103) |
| VL CDRs 18 | TGTSSDVGGYNRVS | EVSNRPS | SSYTSSSTVV |
| | (SEQ ID NO: 104) | (SEQ ID NO: 105) | (SEQ ID NO: 106) |
| VL CDRs 19 | SGSSSNIGSNGVK | RDYQRPS | AAWDDSLNVV |
| | (SEQ ID NO: 107) | (SEQ ID NO: 108) | (SEQ ID NO: 109) |
| VL CDRs 20 | SGSSSNVGSNTVN | RDYQRPS | GAWDDSLNGYV |
| | (SEQ ID NO: 110) | (SEQ ID NO: 111) | (SEQ ID NO: 112) |
| VL CDRs 21 | SGSSSNIGSNTVN | SNNQRPS | AAWDDSLNGPV |
| | (SEQ ID NO: 113) | (SEQ ID NO: 114) | (SEQ ID NO: 115) |
| VL CDRs 22 | TGTGSDVGGYKYVS | DVNYWPS | SSYRTGDTWV |
| | (SEQ ID NO: 116) | (SEQ ID NO: 117) | (SEQ ID NO: 118) |
| VL CDRs 23 | RASQGISNYLA | AASTLQS | QKYNSAPRT |
| | (SEQ ID NO: 119) | (SEQ ID NO: 120) | (SEQ ID NO: 121) |
| VL CDRs 24 | TGTSSDVGGYNYVS | EVSKRPS | SSYAGSNNYV |
| | (SEQ ID NO: 122) | (SEQ ID NO: 123) | (SEQ ID NO: 124) |
| VL CDRs 25 | GGDNIGGKSVH | HDTDRPS | AVWDASLGGSWL |
| | (SEQ ID NO: 125) | (SEQ ID NO: 126) | (SEQ ID NO: 127) |
| VL CDRs 26 | TGSSSNIGAAYDVH | GDSNRPS | QSFDSSLSGSRV |
| | (SEQ ID NO: 128) | (SEQ ID NO: 129) | (SEQ ID NO: 130) |
| VL CDRs 27 | SGSSSNIGSNPVH | RNNQRPS | AAWDVSLSGVV |
| | (SEQ ID NO: 131) | (SEQ ID NO: 132) | (SEQ ID NO: 133) |

Preferably, the antigen binding domain comprises CDRs (CDR1, CDR2, and CDR3), or derivatives thereof, selected from the same variable chain. For example, the antigen binding domain may comprise SEQ ID NOs: 5-7; SEQ ID NOs: 8-10; SEQ ID NOs: 11-13; SEQ ID NOs: 14-16; SEQ ID NOs: 17-19; SEQ ID NOs: 20-22; SEQ ID NOs: 23-25; SEQ ID NOs: 26-28; SEQ ID NOs: 29-31; SEQ ID NOs: 32-34; SEQ ID NOs: 35-37; SEQ ID NOs: 38-40; SEQ ID NOs: 41-43; SEQ ID NOs: 44-46; SEQ ID NOs: 47-49; SEQ ID NOs: 50-52; SEQ ID NOs: 53-55; SEQ ID NOs: 56-58; SEQ ID NOs: 59-61; SEQ ID NOs: 62-64; SEQ ID NOs: 65-67; SEQ ID NOs: 68-70; SEQ ID NOs: 71-73; SEQ ID NOs: 74-76; SEQ ID NOs: 77-79; SEQ ID NOs: 80-82; SEQ ID NOs: 83-85; SEQ ID NOs: 86-88; SEQ ID NOs: 89-91; SEQ ID NOs: 92-94; SEQ ID NOs: 95-97; SEQ ID NOs: 98-100; SEQ ID NOs: 101-103; SEQ ID NOs: 104-106; SEQ ID NOs: 107-109; SEQ ID NOs: 110-112; SEQ ID NOs: 113-115; SEQ ID NOs: 116-118; SEQ ID NOs: 119-121; SEQ ID NOs: 122-124; SEQ ID NOs: 125-127; SEQ ID NOs: 128-130; and/or SEQ ID NOs: 131-133, or derivatives thereof.

In preferred embodiments, the antigen recognition domain comprises a combination variable heavy and variable light CDRs as follows:
(i) SEQ ID NOs: 5-7 and SEQ ID NOs: 17-19, or derivatives thereof;
(ii) SEQ ID NOs: 8-10 and SEQ ID NOs: 20-22, or derivatives thereof;
(iii) SEQ ID NOs: 11-13 and SEQ ID NOs: 23-25, or derivatives thereof;
(iv) SEQ ID NOs: 14-16 and SEQ ID NOs: 26-28, or derivatives thereof;
(v) SEQ ID NOs: 14-16 and SEQ ID NOs: 29-31, or derivatives thereof;
(vi) SEQ ID NOs: 14-16 and SEQ ID NOs: 32-34, or derivatives thereof;
(vii) SEQ ID NOs: 14-16 and SEQ ID NOs: 35-37, or derivatives thereof;
(viii) SEQ ID NOs: 14-16 and SEQ ID NOs: 38-40, or derivatives thereof;
(ix) SEQ ID NOs: 14-16 and SEQ ID NOs: 41-43, or derivatives thereof;
(x) SEQ ID NOs: 14-16 and SEQ ID NOs: 44-46, or derivatives thereof;
(xi) SEQ ID NOs: 14-16 and SEQ ID NOs: 47-49, or derivatives thereof;
(xii) SEQ ID NOs: 14-16 and SEQ ID NOs: 50-52, or derivatives thereof;
(xiii) SEQ ID NOs: 14-16 and SEQ ID NOs: 53-55, or derivatives thereof;
(xiv) SEQ ID NOs: 14-16 and SEQ ID NOs: 56-58, or derivatives thereof;
(xv) SEQ ID NOs: 14-16 and SEQ ID NOs: 59-61, or derivatives thereof;
(xvi) SEQ ID NOs: 62-64 and SEQ ID NOs: 98-100, or derivatives thereof;
(xvii) SEQ ID NOs: 65-67 and SEQ ID NOs: 101-103, or derivatives thereof;
(xviii) SEQ ID NOs: 68-70 and SEQ ID NOs: 104-106, or derivatives thereof;
(xix) SEQ ID NOs: 71-73 and SEQ ID NOs: 107-109, or derivatives thereof;
(xx) SEQ ID NOs: 74-76 and SEQ ID NOs: 110-112, or derivatives thereof;
(xxi) SEQ ID NOs: 77-79 and SEQ ID NOs: 113-115, or derivatives thereof;
(xxii) SEQ ID NOs: 80-82 and SEQ ID NOs: 116-118, or derivatives thereof;
(xxiii) SEQ ID NOs: 83-85 and SEQ ID NOs: 119-121, or derivatives thereof;
(xxiv) SEQ ID NOs: 86-88 and SEQ ID NOs: 122-124, or derivatives thereof;
(xxv) SEQ ID NOs: 89-91 and SEQ ID NOs: 125-127, or derivatives thereof;
(xxvi) SEQ ID NOs: 92-94 and SEQ ID NOs: 128-130, or derivatives thereof; or
(xxvii) SEQ ID NOs: 95-97 and SEQ ID NOs: 131-133, or derivatives thereof.

The antigen binding domain may comprise or consist of a variable heavy domain selected from SEQ ID NOs: 134-149 or a variant which is at least 80% identical to one or more of SEQ ID NOs: 134-149. The variant may be at least 85%, at least 90%, at least 95%, at least 97%, at least 98% or at least 99% identical to one or more of SEQ ID NOs: 134-149.

| Name | Sequence |
|---|---|
| Variable heavy domain 1 | VQLVQSGGGVVQPGGSLRVSCAASGVTLSDYGMHWVRQAP |
| (SEQ ID NO: 134) | GKGLEWMAFIRNDGSDKYYADSVKGRFTISRDNSKKTVSLQ |
| | MSSLRAEDTAVYYCAKNGESGPLDYWYFDLWGRGTLVTV |
| Variable heavy domain 2 | QVQLVQSGGGVVQPGGSLRVSCAASGVTLSDYGMHWVRQ |
| (SEQ ID NO: 135) | APGKGLEWVAFIRNDGSDKYYADSVKGRFTISRDNSEKTVSL |
| | QMSSLRAEDTAVYYCAKNGESGPLDYWYLDLWGRGT |
| Variable heavy domain 3 | QVQLVQSGGGVVQPGGSMRVSCAASGVTLSDYGMHWVRQ |
| (SEQ ID NO: 136) | APGKGLEWVAFIRNDGSDKYYADSVRGRFTISRDNSKKTVFL |
| | QMNSLRAEDTAVYYCAKNGESGPLDYWYFDLWGRGT |
| Variable heavy domain 4 | QVQLVQSGGGVVQPGGSLRVSCAASGVTLSDYGMHWVRQ |
| (SEQ ID NO: 137) | APGKGLEWMAFIRNDGSDKYYADSVKGRFTISRDNSKKTVSL |
| | QMSSLRAEDTAVYYCAKNGESGPLDYWYFDLWGRGT |
| Variable heavy domain 5 | EVQLVQSGAEVKKPGSSVKVSCKASGGTFSSYAISWVRQAP |
| (SEQ ID NO: 138) | GQGLEWMGGIIPIFGTANYAQKFQGRVTITADESTSTAYMELS |
| | SLRSEDTAVYYCARGGYDSRGSYYYMDVWGKGTTVTVSS |
| Variable heavy domain 6 | QVQLVESGGGVVQPGRSLRLSCAASGFTFSSYAMHWVRQA |
| (SEQ ID NO: 139) | PGKGLEWVAVISYDGSNKYYADSVKGRFTISRDNSKNTLYLQ |
| | MNSLRAEDTAVYYCARDREELLALFGGMDVWGQGTTVTVSS |
| Variable heavy domain 7 | EVQLVQSGAEVKKPGSSVKVSCKASGGTFSSYAISWVRQAP |
| (SEQ ID NO: 140) | GQGLEWMGGIIPIFGTANYAQKFQGRVTITADKSTSTAYMELS |
| | SLRSEDTAVYYCARPQSRWLQSGDAFDIWGQGTMVTVSS |
| Variable heavy domain 8 | QVQLQQSGAEVKKPGASVKVSCKASGYTFTSYAMHWVRQA |
| (SEQ ID NO: 141) | PGQRLEWMGRINAGNGNTKYSQKFQGRVTITRDTSASTAYM |
| | ELSSLRSEDTAVYYCARDLTGTLLFDYWGQGTLVTVSS |
| Variable heavy domain 9 | QVQLVQSGAEVKKPGSSVKVSCKASGGTFSSYAISWVRQAP |
| (SEQ ID NO: 142) | GQGLEWMGGIIPIFGTANYAQKFQGRATITADESTSTAYMELS |
| | SLRSEDTAVYYCARRAERWLHLSGAFDIWGQGTMVTVSS |
| Variable heavy domain 10 | QVQLVQSGGGLVQPGGSLRLSCAASGFTFSSYAMSWVRQA |
| (SEQ ID NO: 143) | PGKGLEWVSAISGSGGSTYYADSVKGRFTMSRDNAKNSLYL |
| | QMNSLRVEDSAVYYCATGHYGDYVWGQGALVTVSS |
| Variable heavy domain 11 | QVQLQQSGAEVKKPGASVKVSCKASGYTFTSYAMHWVRQA |
| (SEQ ID NO: 144) | PGQRLEWMGWINAGNGNTKYSQKFQGRVTITRDTSASTAYM |
| | ELSSLRSEDTAVYYCARVSSGGAFDIWGQGTVVTVSS |
| Variable heavy domain 12 | QVQLVQSGAEVKKPGSSVKVSCKASGGTFSSYGFSWVRQA |
| (SEQ ID NO: 145) | PGQGLEWMGEIIPMFGTANYAQKLQGRVTITAETSTSTVYME |
| | LSSLRSEDTATYYCARVPRSSSGYNYGMDVWGQGTTVTVSS |
| Variable heavy domain 13 | QVQLQQSGPGLLKPSQTLSLTCAVSGDSVSTNSGAWSWIRQ |
| (SEQ ID NO: 146) | SPSRGLEWLGRTYYRSKWSTDYALSLQSRVTIKSDRSKNQF |
| | SLQLDSVTPEDTAIYYCARENWNSGGFDYWGQGTLVTVPS |
| Variable heavy domain 14 | EVQLVESGAEVKKPGSSVKVSCKASGGTFSSYAISWVRQAP |
| (SEQ ID NO: 147) | GQGLEWMGGIIPIFGTANYAQKFQGRVTITADKSTSTAYMELS |
| | SLRSEDTAVYYCARAASRWEPGDAFDIWGQGTMVTVSS |
| Variable heavy domain 15 | QVQLVQSGAEVKKPGASVKVSCKTSGYTFTSYDISWVRQAP |
| (SEQ ID NO: 148) | GQGLEWMGWISAYNGNTNYAQKLQGRVTMTTDTSTSTAYM |
| | ELRRLRSDDTAVYYCARGGRWLRSASSFDYWGQGTLVTVS |
| | S |
| Variable heavy domain 16 | EVQLVESGGGLVKPGGSLRLSCAASGFTFSDHYMSWVRQA |
| (SEQ ID NO: 149) | PGKGLEWVSYITSGGSSIYYADSVKGRFTISRDNAKNSLYLQ |
| | MNSLRAEDTAVYYCARGLDSSAYQGRAFDIWGQGTMVTVSS |

The antigen binding domain may comprise or consist of a variable light domain selected from SEQ ID NOs: 150-176 or a variant which is at least 80% identical to one or more of SEQ ID NOs: 150-176. The variant may be at least 85%, at least 90%, at least 95%, at least 97%, at least 98% or at least 99% identical to one or more of SEQ ID NOs: 150-176.

| Name | Sequence |
|---|---|
| Variable light domain 1 | DVVMTQSPSSLSASVGDRVTITCQASQDISNYLNWYQQKPGK |
| (SEQ ID NO: 150) | APKLLIYDASNLETGVPSRFSGSGSGTDFTFTISSLQPEDIATYY |
| | CQQYDNLPPTFGGGTKLTVLG |
| Variable light domain 2 | DVVMTQSPSSLSASVGDRVTITCQSSLDISHYLNWYQQKPGKA |
| (SEQ ID NO: 151) | PKLLIYDASNLETGVPSRFSGSGSGTHFTFTISSLQPEDFATYY |
| | CQQYDNLPLTFGGGTKLEIK |
| Variable light domain 3 | DIVLMQSPSFLSASVGDRVTITCRASHGINNYLAWYQQKPGKA |
| (SEQ ID NO: 152) | PKLLIYAASTLQSGVPSRFSGSGSGTEFTLTISSLQPEDFATYY |
| | CQQYDSYPPTFGRTKVEIKR |
| Variable light domain 4 | DVVMTQSPSSLSASVGDRVTITCQASQDISNYLNWYQQKPGK |
| (SEQ ID NO: 153) | APKLLIYDASNLETGVPSRFSGSGSGTDFTFTISSLQPEDFATY |
| | YCQQYSSFPLTFGGGTKVDIK |
| Variable light domain 5 | DVVMTQSPSSLSASVGDRVTITCQASQDISNYLNWYQQEPGK |
| (SEQ ID NO: 154) | APKLLIYDETHLDSGVPSRFTGSRSGTDFTLTISSLQPEDFATY |
| | YCQQYDSLPPTFGGGTKVDIK |
| Variable light domain 6 | DVVMTQSPSSLSASVGDRVTITCQASQDISNYLNWYQQKPGK |
| (SEQ ID NO: 155) | APKLLIYDASNLETGVPSRFSGSGSGTDFTFTISSLQPEDIATYY |
| | CQQYDNLPITFGGGTKVDIK |
| Variable light domain 7 | DVVMTQSPSSLSASVGDRVTITCQASQDISNYLNWYQQKPGK |
| (SEQ ID NO: 156) | APKLLIYDASNLETGVPSRFSGSGSGTDFTFTISSLQPEDIATYY |
| | CQQYDNLPSTFGGGTKVDIK |
| Variable light domain 8 | DVVMTQSPSSLSASVGDRVTITCQASQDISNYLNWYQQKPGK |
| (SEQ ID NO: 157) | APKLLIYDASNLETGVPSRFSGSGSGTDFTFTISSLQPEDFGTY |
| | YCQQYNTYPLTFGGGTKVDIK |
| Variable light domain 9 | DVVMTQSPSSLTASVGDRVTITCQASQDISNYLNWYQQKPGK |
| (SEQ ID NO: 158) | APKLLIYDASNLETGVPSRFSGSGSGTDFTLSIDSLQPEDFATY |
| | YCQQYHTYPLTFGGGTKVDIK |
| Variable light domain 10 | DVVMTQSPSSLSASVGDRVTITCQASQDISNYLNWYQQKPGK |
| (SEQ ID NO: 159) | APKLLIYDASNLETGVPSRFSGSGSGTDFTFTISSLQPEDIATYY |
| | CQQYDNLPLTFGGGTKVDIK |
| Variable light domain 11 | DVVMTQSPSSLSASVGDRVTITCRTSQGISSALAWYQQKPGK |
| (SEQ ID NO: 160) | APKLLIYDASSLESGVPSRFSGSGSGTDFTLTISSLQPEDFATY |
| | YCQQFNNYPLTFGGGTKVDIK |
| Variable light domain 12 | DVVMTQSPSSLSASVGDRVTITCQASQDISNYLAWYQQKPGR |
| (SEQ ID NO: 161) | APTLLIFAASNLQSGVPSRFSGSGSGTEFTLTISGLQPEDFATY |
| | YCLQDSSYPPTFGGGTKVDIK |
| Variable light domain 13 | DVVMTQSPSTLSASVGDRVTITCRASQSISSWLAWYQQKPGR |
| (SEQ ID NO: 162) | APTLLIYKASNLQSGVPSRFSGSGSGTEFTLTISSLQPDDFASY |
| | YCQQYSNYPLTFGGGTKVDIK |
| Variable light domain 14 | DVVMTQSPSFLSASVGDRVTITCRASHGISNYFAWYQQKPGK |
| (SEQ ID NO: 163) | APKLLIYATSTLQSGVPSRFSGSGSGTEFTLTISGLQPEDFATY |
| | YCQQYSSYPLTFGGGTKVDIK |
| Variable light domain 15 | DVVMTQSPSTLSAYVGDRITITCRASRGISNYLAWYQQKPGKA |
| (SEQ ID NO: 164) | PKLLIYATSTLQSGVPLRFSGSGSGTEFTLTISGLQPEDFATYY |
| | CQQYDSYPPTFGGGTKVDIK |
| Variable light domain 16 | QSVLTQPPSTSGTPGQRVTISCSGSSSNIGGNAVNWYQHFPG |
| (SEQ ID NO: 165) | TAPTLLIYSNNQRPSGVPERFSGSKSGTSASLTVSGLQAEDEA |
| | DYYCTAWDDSLRGYLFGTGTKVTVL |
| Variable light domain 17 | QPVLTQPSSVSVAPGQTARITCGGNNIGSKSVHWYQQKPGQA |
| (SEQ ID NO: 166) | PVLVVYDDSDRPSGIPERFSGSNSGNTATLTISRVEARDEADY |
| | YCHVWDAKTNHQVFGGGTRLTVQ |
| Variable light domain 18 | QPVLTQPRSVSGSPGQSVTISCTGTSSDVGGYNRVSWYQQTP |
| (SEQ ID NO: 167) | GTAPKLMIYEVSNRPSGVSNRFSGSKSGNTASLTISGLQAEDE |
| | ADYYCSSYTSSSTVVFGGGTKLTVL |
| Variable light domain 19 | QSVLTQPPSASGTPGQRVTISCSGSSSNIGSNGVKWYQQLPG |
| (SEQ ID NO: 168) | TAPKLVIYRDYQRPSGVPDRFSGSKSGTSASLAISGLQSEDEA |
| | KYYCAAWDDSLNVVFGGGTQLTVL |
| Variable light domain 20 | QPVLTQSSSASGTPGQRVAISCSGSSSNVGSNTVNWYQQSP |
| (SEQ ID NO: 169) | GTAPKLLISSNHQRPSGVPDRFSGSKFGTSASLAISGLQSEDE |
| | ADYYCGAWDDSLNGYVFGSGTKVTVL |
| Variable light domain 21 | QAGLTQPPSASGTPGQRVTISCSGSSSNIGSNTVNWYQQLPG |
| (SEQ ID NO: 170) | TAPKLLIYSNNQRPSGVPDRFSGSKSGTSASLAISGLQSEDEA |
| | DYYCAAWDDSLNGPVFGGGTKLTVL |
| Variable light domain 22 | QSALTQPASVSGSPGQSITISCTGTGSDVGGYKYVSWYQHHP |
| (SEQ ID NO: 171) | GKAPRLIIYDVNYWPSGVSHRFSGSKSGNTASLTISGLQSEDE |
| | ADYYCSSYRTGDTWVFGGGTKLTVL |
| Variable light domain 23 | DIQMTQSPSSLSASVGDRVTITCRASQGISNYLAWYQQKPGKV |
| (SEQ ID NO: 172) | PKLLIYAASTLQSGVPSRFSGSGSGTDFTLTISSLQPEDVATYY |
| | CQKYNSAPRTFGQGTKVEIK |
| Variable light domain 24 | QPVLTQSSSASGSPGQSVTISCTGTSSDVGGYNYVSWYQQHP |
| (SEQ ID NO: 173) | GKAPKLMIYEVSKRPSGVPDRFSGSKSGSTASLTVSGLQAEDE |
| | AEYYCSSYAGSNNYVFGTGTKVTVL |
| Variable light domain 25 | QPVLTQSSSVSVAPGKTARVTCGGDNIGGKSVHWYQQRAGQ |
| (SEQ ID NO: 174) | APVLVISHDTDRPSGIPERFSGSKSGTSASLAISGLRSEDEADY |
| | YCAVWDASLGGSWLFGGGTKLTVL |
| Variable light domain 26 | AGLTQPPSVSGAPGQRVTISCTGSSSNIGAAYDVHWYQQLPG |
| (SEQ ID NO: 175) | AAPKLLIFGDSNRPSGVPDRFSGSKSDTSASLAITGLQAEDEAD |
| | YYCQSFDSSLSGSRVFGGGTKLTVL |
| Variable light domain 27 | LPVLTQPPSASGTPGQRVTISCSGSSSNIGSNPVHWYQQLPGT |
| (SEQ ID NO: 176) | APKLLVYRNNQRPSGVPDRFSGSKSGTSASLAISGLRSEDEAD |
| | YYCAAWDVSLSGVVFGGGTKLTVL |

Suitably, the antigen recognition domain comprises a combination of a variable heavy domain and a variable light domain. Preferably, the antigen recognition domain comprises a combination of a variable heavy domain and a variable light domain selected from:
(i) an amino acid sequence which has at least 80%, at least 85%, at least 90%, at least 95%, at least 98%, at least 99%, or 100% identity to SEQ ID NO: 134 and an amino acid sequence which has at least 80%, at least 85%, at least 90%, at least 95%, at least 98%, at least 99%, or 100% identity to SEQ ID NO: 150;
(ii) an amino acid sequence which has at least 80%, at least 85%, at least 90%, at least 95%, at least 98%, at least 99%, or 100% identity to SEQ ID NO: 135 and an amino acid sequence which has at least 80%, at least 85%, at least 90%, at least 95%, at least 98%, at least 99%, or 100% identity to SEQ ID NO: 151;
(iii) an amino acid sequence which has at least 80%, at least 85%, at least 90%, at least 95%, at least 98%, at least 99%, or 100% identity to SEQ ID NO: 136 and an amino acid sequence which has at least 80%, at least 85%, at least 90%, at least 95%, at least 98%, at least 99%, or 100% identity to SEQ ID NO: 152;
(iv) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 153;
(v) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 154;
(vi) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 155;
(vii) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 156;
(viii) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 157;
(ix) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 158;
(x) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 159;
(xi) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 160;
(xii) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 161;
(xiii) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 162;
(xiv) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 163;
(xv) an amino acid sequence which has at least 80%, at least 85%, at least 90%, at least 95%, at least 98%, at least 99%, or 100% identity to SEQ ID NO: 137 and an amino acid sequence which has at least 80%, at least 85%, at least 90%, at least 95%, at least 98%, at least 99%, or 100% identity to SEQ ID NO: 164;
(xvi) an amino acid sequence which has at least 80%, at least 85%, at least 90%, at least 95%, at least 98%, at least 99%, or 100% identity to SEQ ID NO: 138 and an amino acid sequence which has at least 80%, at least 85%, at least 90%, at least 95%, at least 98%, at least 99%, or 100% identity to SEQ ID NO: 165;
(xvii) an amino acid sequence which has at least 80%, at least 85%, at least 90%, at least 95%, at least 98%, at least 99%, or 100% identity to SEQ ID NO: 139 and an amino acid sequence which has at least 80%, at least 85%, at least 90%, at least 95%, at least 98%, at least 99%, or 100% identity to SEQ ID NO: 166;
(xviii) an amino acid sequence which has at least 80%, at least 85%, at least 90%, at least 95%, at least 98%, at least 99%, or 100% identity to SEQ ID NO: 140 and an amino acid sequence which has at least 80%, at least 85%, at least 90%, at least 95%, at least 98%, at least 99%, or 100% identity to SEQ ID NO: 167;
(xix) an amino acid sequence which has at least 80%, at least 85%, at least 90%, at least 95%, at least 98%, at least 99%, or 100% identity to SEQ ID NO: 141 and an amino acid sequence which has at least 80%, at least 85%, at least 90%, at least 95%, at least 98%, at least 99%, or 100% identity to SEQ ID NO: 168;
(xx) an amino acid sequence which has at least 80%, at least 85%, at least 90%, at least 95%, at least 98%, at least 99%, or 100% identity to SEQ ID NO: 142 and an amino acid sequence which has at least 80%, at least 85%, at least 90%, at least 95%, at least 98%, at least 99%, or 100% identity to SEQ ID NO: 169;
(xxi) an amino acid sequence which has at least 80%, at least 85%, at least 90%, at least 95%, at least 98%, at least 99%, or 100% identity to SEQ ID NO: 143 and an amino acid sequence which has at least 80%, at least 85%, at least 90%, at least 95%, at least 98%, at least 99%, or 100% identity to SEQ ID NO: 170;
(xxii) an amino acid sequence which has at least 80%, at least 85%, at least 90%, at least 95%, at least 98%, at least 99%, or 100% identity to SEQ ID NO: 144 and an amino acid sequence which has at least 80%, at least 85%, at least 90%, at least 95%, at least 98%, at least 99%, or 100% identity to SEQ ID NO: 171;
(xxiii) an amino acid sequence which has at least 80%, at least 85%, at least 90%, at least 95%, at least 98%, at least 99%, or 100% identity to SEQ ID NO: 145 and an amino acid sequence which has at least 80%, at least 85%, at least 90%, at least 95%, at least 98%, at least 99%, or 100% identity to SEQ ID NO: 172;
(xxiv) an amino acid sequence which has at least 80%, at least 85%, at least 90%, at least 95%, at least 98%, at least 99%, or 100% identity to SEQ ID NO: 146 and an amino acid sequence which has at least 80%, at least 85%, at least 90%, at least 95%, at least 98%, at least 99%, or 100% identity to SEQ ID NO: 173;
(xxv) an amino acid sequence which has at least 80%, at least 85%, at least 90%, at least 95%, at least 98%, at least 99%, or 100% identity to SEQ ID NO: 147 and an amino acid sequence which has at least 80%, at least 85%, at least 90%, at least 95%, at least 98%, at least 99%, or 100% identity to SEQ ID NO: 174;
(xxvi) an amino acid sequence which has at least 80%, at least 85%, at least 90%, at least 95%, at least 98%, at least 99%, or 100% identity to SEQ ID NO: 148 and an amino acid sequence which has at least 80%, at least 85%, at least 90%, at least 95%, at least 98%, at least 99%, or 100% identity to SEQ ID NO: 175; or
(xxvii) an amino acid sequence which has at least 80%, at least 85%, at least 90%, at least 95%, at least 98%, at least 99%, or 100% identity to SEQ ID NO: 149 and an amino acid sequence which has at least 80%, at least 85%, at least 90%, at least 95%, at least 98%, at least 99%, or 100% identity to SEQ ID NO: 176.

The antigen binding domain may comprise or consist of an amino acid sequence selected from SEQ ID NOs: 177-203 or a variant which is at least 80% identical to one or more of SEQ ID NOs: 177-203. The variant may be at least 85%, at least 90%, at least 95%, at least 97%, at least 98% or at least 99% identical to one or more of SEQ ID NOs: 177-203. The antigen binding domain may comprise a linker sequence of (X)n, where X is any amino acid and n is an integer of between 1 and 30. The linker sequence may be any linker sequence known in the art.

Illustrative antigen binding domain 1 (SEQ ID NO: 177, 251-279):
QVQLVQSGGGVVQPGGSLRVSCAASGVTLSDYGMHWVRQAPGKGLEWVAFIRNDGSDK
YYADSVKGRFTISRDNSEKTVSLQMSSLRAEDTAVYYCAKNGESGPLDYWYLDLWG-(X)n-
DVVMTQSPSSLSASVGDRVTITCQSSLDISHYLNWYQQKPGKAPKLLIYDASNLETGVPSRF
SGSGSGTHFTFTISSLQPEDFATYYCQQYDNLPLTFGGGTKLEIK
Illustrative antigen binding domain 2 (SEQ ID NO: 178):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDIATYYCQQYDNLPPTFGGGTKLTVLG
Illustrative antigen binding domain 3 (SEQ ID NO: 179, 280-308):
QVQLVQSGGGVVQPGGSMRVSCAASGVTLSDYGMHWVRQAPGKGLEWVAFIRNDGSDK
YYADSVRGRFTISRDNSKKTVFLQMNSLRAEDTAVYYCAKNGESGPLDYWYFDLWGRGT-
(X)n-
DIVLMQSPSFLSASVGDRVTITCRASHGINNYLAWYQQKPGKAPKLLIYAASTLQSGVPSRF
SGSGSGTEFTLTISSLQPEDFATYYCQQYDSYPPTFGRTKVEIKR
Illustrative antigen binding domain 4 (SEQ ID NO: 180, 309-337):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDFATYYCQQYSSFPLTFGGGTKVDIK
Illustrative antigen binding domain 5 (SEQ ID NO: 181,338-366):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQEPGKAPKLLIYDETHLDSGVPSR
FTGSRSGTDFTLTISSLQPEDFATYYCQQYDSLPPTFGGGTKVDIK
Illustrative antigen binding domain 6 (SEQ ID NO: 182, 367-395):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDIATYYCQQYDNLPITFGGGTKVDIK
Illustrative antigen binding domain 7 (SEQ ID NO: 183, 396-424):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDIATYYCQQYDNLPSTFGGGTKVDIK
Illustrative antigen binding domain 8 (SEQ ID NO: 184, 425-453):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDFGTYYCQQYNTYPLTFGGGTKVDIK
Illustrative antigen binding domain 9 (SEQ ID NO: 185, 454-482):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLTASVGDRVTITCQASQDISNYLNWYQQKPGKAPKLLIYDASNLETGVPSR
FSGSGSGTDFTLSIDSLQPEDFATYYCQQYHTYPLTFGGGTKVDIK
Illustrative antigen binding domain 10 (SEQ ID NO: 186, 483-511):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLNWYQQKPGKAPKLLIYDASNLETGVPSR
FSGSGSGTDFTFTISSLQPEDIATYYCQQYDNLPLTFGGGTKVDIK
Illustrative antigen binding domain 11 (SEQ ID NO: 187, 512-540):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCRTSQGISSALAWYQQKPGKAPKLLIYDASSLESGVPSR
FSGSGSGTDFTLTISSLQPEDFATYYCQQFNNYPLTFGGGTKVDIK
Illustrative antigen binding domain 12 (SEQ ID NO: 188, 541-569):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSSLSASVGDRVTITCQASQDISNYLAWYQQKPGRAPTLLIFAASNLQSGVPSR
FSGSGSGTEFTLTISGLQPEDFATYYCLQDSSYPPTFGGGTKVDIK
Illustrative antigen binding domain 13 (SEQ ID NO: 189, 570-598):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSTLSASVGDRVTITCRASQSISSWLAWYQQKPGRAPTLLIYKASNLQSGVPSR
FSGSGSGTEFTLTISSLQPDDFASYYCQQYSNYPLTFGGGTKVDIK
Illustrative antigen binding domain 14 (SEQ ID NO: 190, 599-627):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSFLSASVGDRVTITCRASHGISNYFAWYQQKPGKAPKLLIYATSTLQSGVPSR
FSGSGSGTEFTLTISGLQPEDFATYYCQQYSSYPLTFGGGTKVDIK
Illustrative antigen binding domain 15 (SEQ ID NO: 191,628-656):
QVQLVQSGGGVVQPGGSLRVSCAASGVTLSDYGMHWVRQAPGKGLEWMAFIRNDGSDK
YYADSVKGRFTISRDNSKKTVSLQMSSLRAEDTAVYYCAKNGESGPLDYWYFDLWGRGT-
(X)n-
DVVMTQSPSTLSAYVGDRITITCRASRGISNYLAWYQQKPGKAPKLLIYATSTLQSGVPLRF
SGSGSGTEFTLTISGLQPEDFATYYCQQYDSYPPTFGGGTKVDIK
Illustrative antigen binding domain 16 (SEQ ID NO: 192, 657-685):
EVQLVQSGAEVKKPGSSVKVSCKASGGTFSSYAISWVRQAPGQGLEWMGGIIPIFGTANYA
QKFQGRVTITADESTSTAYMELSSLRSEDTAVYYCARGGYDSRGSYYYMDVWGKGTTVTV
SS-(X)n-
QSVLTQPPSTSGTPGQRVTISCSGSSSNIGGNAVNWYQHFPGTAPTLLIYSNNQRPSGVPE
RFSGSKSGTSASLTVSGLQAEDEADYYCTAWDDSLRGYLFGTGTKVTVL
Illustrative antigen binding domain 17 (SEQ ID NO: 193, 686-714):
QVQLVESGGGVVQPGRSLRLSCAASGFTFSSYAMHWVRQAPGKGLEWVAVISYDGSNKY
YADSVKGRFTISRDNSKNTLYLQMNSLRAEDTAVYYCARDREELLALFGGMDVWGQGTTV
TVSS-(X)n-
QPVLTQPSSVSVAPGQTARITCGGNNIGSKSVHWYQQKPGQAPVLVVYDDSDRPSGIPER
FSGSNSGNTATLTISRVEARDEADYYCHVWDAKTNHQVFGGGTRLTVQ
Illustrative antigen binding domain 18 (SEQ ID NO: 194, 715-743):
EVQLVQSGAEVKKPGSSVKVSCKASGGTFSSYAISWVRQAPGQGLEWMGGIIPIFGTANYA
QKFQGRVTITADKSTSTAYMELSSLRSEDTAVYYCARPQSRWLQSGDAFDIWGQGTMVTV
SS-(X)n-
QPVLTQPRSVSGSPGQSVTISCTGTSSDVGGYNRVSWYQQTPGTAPKLMIYEVSNRPSGV
SNRFSGSKSGNTASLTISGLQAEDEADYYCSSYTSSSTVVFGGGTKLTVL
Illustrative antigen binding domain 19 (SEQ ID NO: 195, 744-772):
QVQLQQSGAEVKKPGASVKVSCKASGYTFTSYAMHWVRQAPGQRLEWMGRINAGNGNT
KYSQKFQGRVTITRDTSASTAYMELSSLRSEDTAVYYCARDLTGTLLFDYWGQGTLVTVSS
-(X)n-
QSVLTQPPSASGTPGQRVTISCSGSSSNIGSNGVKWYQQLPGTAPKLVIYRDYQRPSGVP
DRFSGSKSGTSASLAISGLQSEDEAKYYCAAWDDSLNVVFGGGTQLTVL
Illustrative antigen binding domain 20 (SEQ ID NO: 196, 773-801):
QVQLVQSGAEVKKPGSSVKVSCKASGGTFSSYAISWVRQAPGQGLEWMGGIIPIFGTANY
AQKFQGRATITADESTSTAYMELSSLRSEDTAVYYCARRAERWLHLSGAFDIWGQGTMVT
VSS-(X)n-
QPVLTQSSSASGTPGQRVAISCSGSSSNVGSNTVNWYQQSPGTAPKLLISSNHQRPSGVP
DRFSGSKFGTSASLAISGLQSEDEADYYCGAWDDSLNGYVFGSGTKVTVL
Illustrative antigen binding domain 21 (SEQ ID NO: 197, 802-830):
QVQLVQSGGGLVQPGGSLRLSCAASGFTFSSYAMSWVRQAPGKGLEWVSAISGSGGSTY
YADSVKGRFTMSRDNAKNSLYLQMNSLRVEDSAVYYCATGHYGDYVWGQGALVTVSS-
(X)n-
QAGLTQPPSASGTPGQRVTISCSGSSSNIGSNTVNWYQQLPGTAPKLLIYSNNQRPSGVPD
RFSGSKSGTSASLAISGLQSEDEADYYCAAWDDSLNGPVFGGGTKLTVL
Illustrative antigen binding domain 22 (SEQ ID NO: 198, 831-859):
QVQLQQSGAEVKKPGASVKVSCKASGYTFTSYAMHWVRQAPGQRLEWMGWINAGNGNT
KYSQKFQGRVTITRDTSASTAYMELSSLRSEDTAVYYCARVSSGGAFDIWGQGTVVTVSS-
(X)n-
QSALTQPASVSGSPGQSITISCTGTGSDVGGYKYVSWYQHHPGKAPRLIIYDVNYWPSGVS
HRFSGSKSGNTASLTISGLQSEDEADYYCSSYRTGDTWVFGGGTKLTVL
Illustrative antigen binding domain 23 (SEQ ID NO: 199, 860-888):
QVQLVQSGAEVKKPGSSVKVSCKASGGTFSSYGFSWVRQAPGQGLEWMGEIIPMFGTAN
YAQKLQGRVTITAETSTSTVYMELSSLRSEDTATYYCARVPRSSSGYNYGMDVWGQGTTV
TVSS-(X)n-
DIQMTQSPSSLSASVGDRVTITCRASQGISNYLAWYQQKPGKVPKLLIYAASTLQSGVPSRF
SGSGSGTDFTLTISSLQPEDVATYYCQKYNSAPRTFGQGTKVEIK
Illustrative antigen binding domain 24 (SEQ ID NO: 200, 889-917):
QVQLQQSGPGLLKPSQTLSLTCAVSGDSVSTNSGAWSWIRQSPSRGLEWLGRTYYRSKW
STDYALSLQSRVTIKSDRSKNQFSLQLDSVTPEDTAIYYCARENWNSGGFDYWGQGTLVTV
PS-(X)n-
QPVLTQSSSASGSPGQSVTISCTGTSSDVGGYNYVSWYQQHPGKAPKLMIYEVSKRPSGV
PDRFSGSKSGSTASLTVSGLQAEDEAEYYCSSYAGSNNYVFGTGTKVTVL
Illustrative antigen binding domain 25 (SEQ ID NO: 201,918-946):
EVQLVESGAEVKKPGSSVKVSCKASGGTFSSYAISWVRQAPGQGLEWMGGIIPIFGTANYA
QKFQGRVTITADKSTSTAYMELSSLRSEDTAVYYCARAASRWEPGDAFDIWGQGTMVTVS
S-(X)n-
QPVLTQSSSVSVAPGKTARVTCGGDNIGGKSVHWYQQRAGQAPVLVISHDTDRPSGIPER
FSGSKSGTSASLAISGLRSEDEADYYCAVWDASLGGSWLFGGGTKLTVL
Illustrative antigen binding domain 26 (SEQ ID NO: 202, 947-975):
QVQLVQSGAEVKKPGASVKVSCKTSGYTFTSYDISWVRQAPGQGLEWMGWISAYNGNTN
YAQKLQGRVTMTTDTSTSTAYMELRRLRSDDTAVYYCARGGRWLRSASSFDYWGQGTLV
TVSS-(X)n-
QAGLTQPPSVSGAPGQRVTISCTGSSSNIGAAYDVHWYQQLPGAAPKLLIFGDSNRPSGVP
DRFSGSKSDTSASLAITGLQAEDEADYYCQSFDSSLSGSRVFGGGTKLTVL
Illustrative antigen binding domain 27(SEQ ID NO: 203, 976-1004):
EVQLVESGGGLVKPGGSLRLSCAASGFTFSDHYMSWVRQAPGKGLEWVSYITSGGSSIYY
ADSVKGRFTISRDNAKNSLYLQMNSLRAEDTAVYYCARGLDSSAYQGRAFDIWGQGTMVT
VSS-(X)n-
LPVLTQPPSASGTPGQRVTISCSGSSSNIGSNPVHWYQQLPGTAPKLLVYRNNQRPSGVP
DRFSGSKSGTSASLAISGLRSEDEADYYCAAWDVSLSGVVFGGGTKLTVL

The antigen recognition domain variants described herein retain antigen-binding ability. For example, the variants may be capable of binding HLA-A2 to at least 10%, at least 20%, at least 30%, at least 40%, at least 50%, at least 60%, at least 70%, at least 80%, or at least 90% of the level of the corresponding reference amino acid sequence. The variant may be capable of binding HLA-A2 to a similar or the same level as the corresponding reference amino acid sequence or may be capable of binding HLA-A2 to a greater level than the corresponding reference amino acid sequence (e.g. increased by at least 10%, at least 20%, at least 30%, at least 40% or at least 50%). Accordingly, the antigen recognition domain may comprise or consist of an amino acid sequence comprising one or more (e.g. one, two, three, four, five, or six) of the CDRs of SEQ ID NOs: 5-133 (shown above, underlined in SEQ ID NOs: 134-176).

Substitutions, variations, modifications, replacements, deletions and/or additions of one (or more) amino acid residues may occur in the framework region.

Thus, in some embodiments the antigen recognition domain comprises or consists of:
(i) an amino acid sequence which has at least 80%, at least 85%, at least 90%, at least 95%, at least 98%, at least 99%, or 100% identity to SEQ ID NO: 134, wherein the amino acid sequence comprises CDR1, CDR2 and CDR3 regions consisting of SEQ ID NOs: 5-7, respectively, and/or an amino acid sequence which has at least 80%, at least 85%, at least 90%, at least 95%, at least 98%, at least 99%, or 100% identity to SEQ ID NO: 150, wherein the amino acid sequence comprises CDR1, CDR2 and CDR3 regions consisting of SEQ ID NOs: 17-19, respectively;
(ii) an amino acid sequence which has at least 80%, at least 85%, at least 90%, at least 95%, at least 98%, at least 99%, or 100% identity to SEQ ID NO: 135, wherein the amino acid sequence comprises CDR1, CDR2 and CDR3 regions consisting of SEQ ID NOs: 8-10, respectively, and/or an amino acid sequence which has at least 80%, at least 85%, at least 90%, at least 95%, at least 98%, at least 99%, or 100% identity to SEQ ID NO: 151, wherein the amino acid sequence comprises CDR1, CDR2 and CDR3 regions consisting of SEQ ID NOs: 20-22, respectively;
(iii) an amino acid sequence which has at least 80%, at least 85%, at least 90%, at least 95%, at least 98%, at least 99%, or 100% identity to SEQ ID NO: 136, wherein the amino acid sequence comprises CDR1, CDR2 and CDR3 regions consisting of SEQ ID NOs: 11-13, respectively, and/or an amino acid sequence which has at least 80%, at least 85%, at least 90%, at least 95%, at least 98%, at least 99%, or 100% identity to SEQ ID NO: 152, wherein the amino acid sequence comprises CDR1, CDR2 and CDR3 regions consisting of SEQ ID NOs: 23-25, respectively;
(iv) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 153, wherein the amino acid sequence comprises CDR1, CDR2 and CDR3 regions consisting of SEQ ID NOs: 26-28, respectively;
(v) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 154, wherein the amino acid sequence comprises CDR1, CDR2 and CDR3 regions consisting of SEQ ID NOs: 29-31, respectively;
(vi) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 155, wherein the amino acid sequence comprises CDR1, CDR2 and CDR3 regions consisting of SEQ ID NOs: 32-34, respectively;
(vii) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 156, wherein the amino acid sequence comprises CDR1, CDR2 and CDR3 regions consisting of SEQ ID NOs: 35-37, respectively;
(viii) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 157, wherein the amino acid sequence comprises CDR1, CDR2 and CDR3 regions consisting of SEQ ID NOs: 38-40, respectively;
(ix) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 158, wherein the amino acid sequence comprises CDR1, CDR2 and CDR3 regions consisting of SEQ ID NOs: 41-43, respectively;
(x) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 159, wherein the amino acid sequence comprises CDR1, CDR2 and CDR3 regions consisting of SEQ ID NOs: 44-46, respectively;
(xi) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 160, wherein the amino acid sequence comprises CDR1, CDR2 and CDR3 regions consisting of SEQ ID NOs: 47-49, respectively;
(xii) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 161, wherein the amino acid sequence comprises CDR1, CDR2 and CDR3 regions consisting of SEQ ID NOs: 50-52, respectively;
(xiii) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 162, wherein the amino acid sequence comprises CDR1, CDR2 and CDR3 regions consisting of SEQ ID NOs: 53-55, respectively;
(xiv) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 163, wherein the amino acid sequence comprises CDR1, CDR2 and CDR3 regions consisting of SEQ ID NOs: 56-58, respectively;
(xv) an amino acid sequence which has at least 80%, at least 85%, at least 90%, at least 95%, at least 98%, at least 99%, or 100% identity to SEQ ID NO: 137, wherein the amino acid sequence comprises CDR1, CDR2 and CDR3 regions consisting of SEQ ID NOs: 14-16, respectively, and/or an amino acid sequence which has at least 80%, at least 85%, at least 90%, at least 95%, at least 98%, at least 99%, or 100% identity to SEQ ID NO: 164, wherein the amino acid sequence comprises CDR1, CDR2 and CDR3 regions consisting of SEQ ID NOs: 59-61, respectively;
(xvi) an amino acid sequence which has at least 80%, at least 85%, at least 90%, at least 95%, at least 98%, at least 99%, or 100% identity to SEQ ID NO: 138, wherein the amino acid sequence comprises CDR1, CDR2 and CDR3 regions consisting of SEQ ID NOs: 62-64, respectively, and/or an amino acid sequence which has at least 80%, at least 85%, at least 90%, at least 95%, at least 98%, at least 99%, or 100% identity to SEQ ID NO: 165, wherein the amino acid sequence comprises CDR1, CDR2 and CDR3 regions consisting of SEQ ID NOs: 98-100, respectively;
(xvii) an amino acid sequence which has at least 80%, at least 85%, at least 90%, at least 95%, at least 98%, at least 99%, or 100% identity to SEQ ID NO: 139, wherein the amino acid sequence comprises CDR1, CDR2 and CDR3 regions consisting of SEQ ID NOs: 65-67, respectively, and/or an amino acid sequence which has at least 80%, at least 85%, at least 90%, at least 95%, at least 98%, at least 99%, or 100% identity to SEQ ID NO: 166, wherein the amino acid sequence comprises CDR1, CDR2 and CDR3 regions consisting of SEQ ID NOs: 101-103, respectively;
(xviii) an amino acid sequence which has at least 80%, at least 85%, at least 90%, at least 95%, at least 98%, at least 99%, or 100% identity to SEQ ID NO: 140, wherein the amino acid sequence comprises CDR1, CDR2 and CDR3 regions consisting of SEQ ID NOs: 68-70, respectively, and/or an amino acid sequence which has at least 80%, at least 85%, at least 90%, at least 95%, at least 98%, at least 99%, or 100% identity to SEQ ID NO: 167, wherein the amino acid sequence comprises CDR1, CDR2 and CDR3 regions consisting of SEQ ID NOs: 104-106, respectively;
(xix) an amino acid sequence which has at least 80%, at least 85%, at least 90%, at least 95%, at least 98%, at least 99%, or 100% identity to SEQ ID NO: 141, wherein the amino acid sequence comprises CDR1, CDR2 and CDR3 regions consisting of SEQ ID NOs: 71-73, respectively, and/or an amino acid sequence which has at least 80%, at least 85%, at least 90%, at least 95%, at least 98%, at least 99%, or 100% identity to SEQ ID NO: 168, wherein the amino acid sequence comprises CDR1, CDR2 and CDR3 regions consisting of SEQ ID NOs: 107-109, respectively;
(xx) an amino acid sequence which has at least 80%, at least 85%, at least 90%, at least 95%, at least 98%, at least 99%, or 100% identity to SEQ ID NO: 142, wherein the amino acid sequence comprises CDR1, CDR2 and CDR3 regions consisting of SEQ ID NOs: 74-76, respectively, and/or an amino acid sequence which has at least 80%, at least 85%, at least 90%, at least 95%, at least 98%, at least 99%, or 100% identity to SEQ ID NO: 169, wherein the amino acid sequence comprises CDR1, CDR2 and CDR3 regions consisting of SEQ ID NOs: 110-112, respectively;
(xxi) an amino acid sequence which has at least 80%, at least 85%, at least 90%, at least 95%, at least 98%, at least 99%, or 100% identity to SEQ ID NO: 143, wherein the amino acid sequence comprises CDR1, CDR2 and CDR3 regions consisting of SEQ ID NOs: 77-79, respectively, and/or an amino acid sequence which has at least 80%, at least 85%, at least 90%, at least 95%, at least 98%, at least 99%, or 100% identity to SEQ ID NO: 170, wherein the amino acid sequence comprises CDR1, CDR2 and CDR3 regions consisting of SEQ ID NOs: 113-115, respectively;
(xxii) an amino acid sequence which has at least 80%, at least 85%, at least 90%, at least 95%, at least 98%, at least 99%, or 100% identity to SEQ ID NO: 144, wherein the amino acid sequence comprises CDR1, CDR2 and CDR3 regions consisting of SEQ ID NOs: 80-82, respectively, and/or an amino acid sequence which has at least 80%, at least 85%, at least 90%, at least 95%, at least 98%, at least 99%, or 100% identity to SEQ ID NO: 171, wherein the amino acid sequence comprises CDR1, CDR2 and CDR3 regions consisting of SEQ ID NOs: 116-118, respectively;
(xxiii) an amino acid sequence which has at least 80%, at least 85%, at least 90%, at least 95%, at least 98%, at least 99%, or 100% identity to SEQ ID NO: 145, wherein the amino acid sequence comprises CDR1, CDR2 and CDR3 regions consisting of SEQ ID NOs: 83-85, respectively, and/or an amino acid sequence which has at least 80%, at least 85%, at least 90%, at least 95%, at least 98%, at least 99%, or 100% identity to SEQ ID NO: 172, wherein the amino acid sequence comprises CDR1, CDR2 and CDR3 regions consisting of SEQ ID NOs: 119-121, respectively;
(xxiv) an amino acid sequence which has at least 80%, at least 85%, at least 90%, at least 95%, at least 98%, at least 99%, or 100% identity to SEQ ID NO: 146, wherein the amino acid sequence comprises CDR1, CDR2 and CDR3 regions consisting of SEQ ID NOs: 86-88, respectively, and/or an amino acid sequence which has at least 80%, at least 85%, at least 90%, at least 95%, at least 98%, at least 99%, or 100% identity to SEQ ID NO: 173, wherein the amino acid sequence comprises CDR1, CDR2 and CDR3 regions consisting of SEQ ID NOs: 122-124, respectively;
(xxv) an amino acid sequence which has at least 80%, at least 85%, at least 90%, at least 95%, at least 98%, at least 99%, or 100% identity to SEQ ID NO: 147, wherein the amino acid sequence comprises CDR1, CDR2 and CDR3 regions consisting of SEQ ID NOs: 89-91, respectively, and/or an amino acid sequence which has at least 80%, at least 85%, at least 90%, at least 95%, at least 98%, at least 99%, or 100% identity to SEQ ID NO: 174, wherein the amino acid sequence comprises CDR1, CDR2 and CDR3 regions consisting of SEQ ID NOs: 125-127, respectively;
(xxvi) an amino acid sequence which has at least 80%, at least 85%, at least 90%, at least 95%, at least 98%, at least 99%, or 100% identity to SEQ ID NO: 148, wherein the amino acid sequence comprises CDR1, CDR2 and CDR3 regions consisting of SEQ ID NOs: 92-94, respectively, and/or an amino acid sequence which has at least 80%, at least 85%, at least 90%, at least 95%, at least 98%, at least 99%, or 100% identity to SEQ ID NO: 175, wherein the amino acid sequence comprises CDR1, CDR2 and CDR3 regions consisting of SEQ ID NOs: 128-130, respectively; or
(xxvii) an amino acid sequence which has at least 80%, at least 85%, at least 90%, at least 95%, at least 98%, at least 99%, or 100% identity to SEQ ID NO: 149, wherein the amino acid sequence comprises CDR1, CDR2 and CDR3 regions consisting of SEQ ID NOs: 95-97, respectively, and/or an amino acid sequence which has at least 80%, at least 85%, at least 90%, at least 95%, at least 98%, at least 99%, or 100% identity to SEQ ID NO: 176, wherein the amino acid sequence comprises CDR1, CDR2 and CDR3 regions consisting of SEQ ID NOs: 131-133, respectively.

In some embodiments the antigen recognition domain comprises or consists of:
(i) an amino acid sequence which has at least 80%, at least 85%, at least 90%, at least 95%, at least 98%, at least 99%, or 100% identity to SEQ ID NO: 177, wherein the variable heavy domain comprises CDR1, CDR2 and CDR3 regions consisting of SEQ ID NOs: 5-7, respectively, and wherein the variable light domain comprises CDR1, CDR2 and CDR3 regions consisting of SEQ ID NOs: 17-19, respectively;
(ii) an amino acid sequence which has at least 80%, at least 85%, at least 90%, at least 95%, at least 98%, at least 99%, or 100% identity to SEQ ID NO: 178, wherein the variable heavy domain comprises CDR1, CDR2 and CDR3 regions consisting of SEQ ID NOs: 8-10, respectively, and wherein the variable light domain comprises CDR1, CDR2 and CDR3 regions consisting of SEQ ID NOs: 20-22, respectively;
(iii) an amino acid sequence which has at least 80%, at least 85%, at least 90%, at least 95%, at least 98%, at least 99%, or 100% identity to SEQ ID NO: 179, wherein the variable heavy domain comprises CDR1, CDR2 and CDR3 regions consisting of SEQ ID NOs: 11-13, respectively, and wherein the variable light domain comprises CDR1, CDR2 and CDR3 regions consisting of SEQ ID NOs: 23-25, respectively;
(iv) an amino acid sequence which has at least 80%, at least 85%, at least 90%, at least 95%, at least 98%, at least 99%, or 100% identity to SEQ ID NO: 180, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 26-28, respectively;
(v) an amino acid sequence which has at least 80%, at least 85%, at least 90%, at least 95%, at least 98%, at least 99%, or 100% identity to SEQ ID NO: 181, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 29-31, respectively;
(vi) an amino acid sequence which has at least 80%, at least 85%, at least 90%, at least 95%, at least 98%, at least 99%, or 100% identity to SEQ ID NO: 182, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 32-34, respectively;
(vii) an amino acid sequence which has at least 80%, at least 85%, at least 90%, at least 95%, at least 98%, at least 99%, or 100% identity to SEQ ID NO: 183, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 35-37, respectively;
(viii) an amino acid sequence which has at least 80%, at least 85%, at least 90%, at least 95%, at least 98%, at least 99%, or 100% identity to SEQ ID NO: 184, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 38-40, respectively;
(ix) an amino acid sequence which has at least 80%, at least 85%, at least 90%, at least 95%, at least 98%, at least 99%, or 100% identity to SEQ ID NO: 185, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 41-43, respectively;
(x) an amino acid sequence which has at least 80%, at least 85%, at least 90%, at least 95%, at least 98%, at least 99%, or 100% identity to SEQ ID NO: 186, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 44-46, respectively;
(xi) an amino acid sequence which has at least 80%, at least 85%, at least 90%, at least 95%, at least 98%, at least 99%, or 100% identity to SEQ ID NO: 187, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 47-49, respectively;
(xii) an amino acid sequence which has at least 80%, at least 85%, at least 90%, at least 95%, at least 98%, at least 99%, or 100% identity to SEQ ID NO: 188, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 50-52, respectively;
(xiii) an amino acid sequence which has at least 80%, at least 85%, at least 90%, at least 95%, at least 98%, at least 99%, or 100% identity to SEQ ID NO: 189, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 53-55, respectively;
(xiv) an amino acid sequence which has at least 80%, at least 85%, at least 90%, at least 95%, at least 98%, at least 99%, or 100% identity to SEQ ID NO: 190, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 56-58, respectively;
(xv) an amino acid sequence which has at least 80%, at least 85%, at least 90%, at least 95%, at least 98%, at least 99%, or 100% identity to SEQ ID NO: 191, wherein the variable heavy domain comprises CDR1, CDR2 and CDR3 regions consisting of SEQ ID NOs: 14-16, respectively, and wherein the variable light domain comprises CDR1, CDR2 and CDR3 regions consisting of SEQ ID NOs: 59-61, respectively;
(xvi) an amino acid sequence which has at least 80%, at least 85%, at least 90%, at least 95%, at least 98%, at least 99%, or 100% identity to SEQ ID NO: 192, wherein the variable heavy domain comprises CDR1, CDR2 and CDR3 regions consisting of SEQ ID NOs: 62-64, respectively, and wherein the variable light domain comprises CDR1, CDR2 and CDR3 regions consisting of SEQ ID NOs: 98-100, respectively;
(xvii) an amino acid sequence which has at least 80%, at least 85%, at least 90%, at least 95%, at least 98%, at least 99%, or 100% identity to SEQ ID NO: 193, wherein the variable heavy domain comprises CDR1, CDR2 and CDR3 regions consisting of SEQ ID NOs: 65-67, respectively, and wherein the variable light domain comprises CDR1, CDR2 and CDR3 regions consisting of SEQ ID NOs: 101-103, respectively;
(xviii) an amino acid sequence which has at least 80%, at least 85%, at least 90%, at least 95%, at least 98%, at least 99%, or 100% identity to SEQ ID NO: 194, wherein the variable heavy domain comprises CDR1, CDR2 and CDR3 regions consisting of SEQ ID NOs: 68-70, respectively, and wherein the variable light domain comprises CDR1, CDR2 and CDR3 regions consisting of SEQ ID NOs: 104-106, respectively;
(xix) an amino acid sequence which has at least 80%, at least 85%, at least 90%, at least 95%, at least 98%, at least 99%, or 100% identity to SEQ ID NO: 195, wherein the variable heavy domain comprises CDR1, CDR2 and CDR3 regions consisting of SEQ ID NOs: 71-73, respectively, and wherein the variable light domain comprises CDR1, CDR2 and CDR3 regions consisting of SEQ ID NOs: 107-109, respectively;
(xx) an amino acid sequence which has at least 80%, at least 85%, at least 90%, at least 95%, at least 98%, at least 99%, or 100% identity to SEQ ID NO: 196, wherein the variable heavy domain comprises CDR1, CDR2 and CDR3 regions consisting of SEQ ID NOs: 74-76, respectively, and wherein the variable light domain comprises CDR1, CDR2 and CDR3 regions consisting of SEQ ID NOs: 110-112, respectively;
(xxi) an amino acid sequence which has at least 80%, at least 85%, at least 90%, at least 95%, at least 98%, at least 99%, or 100% identity to SEQ ID NO: 197, wherein the variable heavy domain comprises CDR1, CDR2 and CDR3 regions consisting of SEQ ID NOs: 77-79, respectively, and wherein the variable light domain comprises CDR1, CDR2 and CDR3 regions consisting of SEQ ID NOs: 113-115, respectively;
(xxii) an amino acid sequence which has at least 80%, at least 85%, at least 90%, at least 95%, at least 98%, at least 99%, or 100% identity to SEQ ID NO: 198, wherein the variable heavy domain comprises CDR1, CDR2 and CDR3 regions consisting of SEQ ID NOs: 80-82, respectively, and wherein the variable light domain comprises CDR1, CDR2 and CDR3 regions consisting of SEQ ID NOs: 116-118, respectively;
(xxiii) an amino acid sequence which has at least 80%, at least 85%, at least 90%, at least 95%, at least 98%, at least 99%, or 100% identity to SEQ ID NO: 199, wherein the variable heavy domain comprises CDR1, CDR2 and CDR3 regions consisting of SEQ ID NOs: 83-85, respectively, and wherein the variable light domain comprises CDR1, CDR2 and CDR3 regions consisting of SEQ ID NOs: 119-121, respectively;
(xxiv) an amino acid sequence which has at least 80%, at least 85%, at least 90%, at least 95%, at least 98%, at least 99%, or 100% identity to SEQ ID NO: 200, wherein the variable heavy domain comprises CDR1, CDR2 and CDR3 regions consisting of SEQ ID NOs: 86-88, respectively, and wherein the variable light domain comprises CDR1, CDR2 and CDR3 regions consisting of SEQ ID NOs: 122-124, respectively;
(xxv) an amino acid sequence which has at least 80%, at least 85%, at least 90%, at least 95%, at least 98%, at least 99%, or 100% identity to SEQ ID NO: 201, wherein the variable heavy domain comprises CDR1, CDR2 and CDR3 regions consisting of SEQ ID NOs: 89-91, respectively, and wherein the variable light domain comprises CDR1, CDR2 and CDR3 regions consisting of SEQ ID NOs: 125-127, respectively;
(xxvi) an amino acid sequence which has at least 80%, at least 85%, at least 90%, at least 95%, at least 98%, at least 99%, or 100% identity to SEQ ID NO: 202, wherein the variable heavy domain comprises CDR1, CDR2 and CDR3 regions consisting of SEQ ID NOs: 92-94, respectively, and wherein the variable light domain comprises CDR1, CDR2 and CDR3 regions consisting of SEQ ID NOs: 128-130, respectively; or (xxvii) an amino acid sequence which has at least 80%, at least 85%, at least 90%, at least 95%, at least 98%, at least 99%, or 100% identity to SEQ ID NO: 203, wherein the variable heavy domain comprises CDR1, CDR2 and CDR3 regions consisting of SEQ ID NOs: 95-97, respectively, and wherein the variable light domain comprises CDR1, CDR2 and CDR3 regions consisting of SEQ ID NOs: 131-133, respectively.

Hinge Domain

The CAR may comprise a hinge domain.

The “hinge domain”, also referred to as the “spacer domain”, as used herein refers to the extracellular part of the CAR that separates the antigen binding domain from the transmembrane domain. The hinge may provide flexibility to access the targeted antigen. For example, long spacers provide extra flexibility to the CAR and allow for better access to membrane-proximal epitopes

Suitable hinge domains will be apparent to those of skill in the art (e.g. Guedan, S., et al., 2018. Molecular Therapy-Methods & Clinical Development, 12, 145-156). Suitable hinge domains include, but are not limited to: CD28 hinge domain, a CD8 hinge domain, an IgG hinge domain, and an IgD hinge domain. Preferably the hinge domain is a CD8 or CD28 hinge domain.

Most preferably, the hinge domain is a CD8 hinge domain. Suitably, the hinge domain may comprise the amino acid sequence shown as SEQ ID NO: 222, or a variant which is at least 80% identical to SEQ ID NO: 222.

Illustrative CD8 hinge domain (SEQ ID NO: 222):
TTTPAPRPPTPAPTIASQPLSLRPEACRPAAGGAVHTRGLDFACD

Suitably, the variant may be at least 85%, at least 90%, at least 95%, at least 97%, at least 98% or at least 99% identical to SEQ ID NO: 222.

Suitably the hinge domain is a CD28 hinge domain. Suitably, the hinge domain may comprise the amino acid sequence shown as SEQ ID NO: 221, or a variant which is at least 80% identical to SEQ ID NO: 221.

Illustrative CD28 hinge domain (SEQ ID NO: 221):
IEVMYPPPYLDNEKSNGTIIHVKGKHLCPSPLFPGPSKP

Suitably, the variant may be at least 85%, at least 90%, at least 95%, at least 97%, at least 98% or at least 99% identical to SEQ ID NO: 221.

Suitably, the CAR may encode a tag, such as a c-Myc tag (EQKLISEEDL—SEQ ID NO: 223). Suitably the tag may be incorporated into the extracellular domain of the CAR, for example in the hinge domain of the extracellular domain. An illustrative CD28 hinge domain with an integrated c-Myc tag is shown below. Suitably, the hinge domain may comprise the amino acid sequence shown as SEQ ID NO: 224, or a variant which is at least 80% identical to SEQ ID NO: 224.

Illustrative CD28 hinge domain with an integrated
c-Myc tag (SEQ ID NO: 224):
IEVEQKLISEEDLLDNEKSNGTIIHVKGKHLCPSPLFPGPSKP

Suitably, the variant may be at least 85%, at least 90%, at least 95%, at least 97%, at least 98% or at least 99% identical to SEQ ID NO: 224.

Transmembrane Domain

The CAR may comprise a transmembrane domain.

The “transmembrane domain” as used herein refers to the part of the CAR that anchors the CAR into the cell membrane of the Treg. Thus, the transmembrane domain is capable of spanning or being present within the cell membrane of the Treg. The transmembrane domain may be derived from a protein comprising an extracellular and/or intracellular portions and thus the transmembrane domain as used herein may be attached to extracellular and/or intracellular residues derived from the protein of origin, in addition to the portion within or spanning the cell membrane. For example, the transmembrane domain may be attached to a hinge domain derived from the protein of origin e.g. a transmembrane domain derived from CD8 may be attached to a hinge domain derived from CD8. Further, a transmembrane domain derived from CD8 or CD28 for example, may be attached to a CD8 or CD28 costimulatory domain. It will be appreciated by a skilled person that the transmembrane domain can also be synthetic, e.g. de novo designed and not derived from a protein having a transmembrane domain. The presence of a transmembrane domain within a cell membrane can be assessed using any suitable method known in the art, including fluorescence labelling with fluorescence microscopy.

Suitable transmembrane domains will be apparent to those of skill in the art. The transmembrane domain may comprise the transmembrane sequence from any protein which has a transmembrane domain, including any of the type I, type II or type III transmembrane proteins. The transmembrane domain of the CAR may also comprise an artificial hydrophobic sequence. The transmembrane domain may be selected so as not to dimerize.

Examples of transmembrane (TM) domains used in CAR constructs are: 1) The CD28 TM domain (Pule et al, Mol Ther, 2005, November; 12(5):933-41; Brentjens et al, CCR, 2007, Sep. 15; 13 (18 Pt 1):5426-35; Casucci et al, Blood, 2013, Nov. 14; 122(20):3461-72.); 2) The OX40 TM domain (Pule et al, Mol Ther, 2005, November; 12(5):933-41); 3) The 41BB TM domain (Brentjens et al, CCR, 2007, Sep. 15; 13 (18 Pt 1):5426-35); 4) The CD3 zeta TM domain (Pule et al, Mol Ther, 2005, November; 12(5):933-41; Savoldo B, Blood, 2009, Jun. 18; 113(25):6392-402.); 5) The CD8 alpha TM domain (Maher et al, Nat Biotechnol, 2002, January; 20(1):70-5.; Imai C, Leukemia, 2004, April; 18(4):676-84; Brentjens et al, CCR, 2007, Sep. 15; 13 (18 Pt 1):5426-35; Milone et al, Mol Ther, 2009, August; 17(8):1453-64.); 6) the ICOS TM domain; 7) the CD4 TM domain.

Most preferably, the CAR may comprise a CD8 transmembrane domain. Suitably, the transmembrane domain may comprise the amino acid sequence shown as SEQ ID NO: 225, or a variant which is at least 80% identical to SEQ ID NO: 225.

Illustrative CD8 TM domain (AA 183 to 203) (SEQ
ID NO: 225):
IYIWAPLAGTCGVLLLSLVIT

Suitably, the variant may be at least 85%, at least 90%, at least 95%, at least 97%, at least 98% or at least 99% identical to SEQ ID NO: 225.

Most preferably, the CAR may comprise a CD8 hinge domain and a CD8 transmembrane domain. Suitably, the hinge and transmembrane domain may comprise the amino acid sequence shown as SEQ ID NO: 226, or a variant which is at least 80% identical to SEQ ID NO: 226.

Illustrative CD8 hinge domain and CD8 transmem-
brane domain (SEQ ID NO: 226):
TTTPAPRPPTPAPTIASQPLSLRPEACRPAAGGAVHTRGLDFACDIYIWA
PLAGTCGVLLLSLVIT

Suitably, the variant may be at least 85%, at least 90%, at least 95%, at least 97%, at least 98% or at least 99% identical to SEQ ID NO: 226.

Suitably, the CAR may comprise a CD28 hinge domain and a CD8 transmembrane domain. Suitably, the hinge and transmembrane domain may comprise the amino acid sequence shown as SEQ ID NO: 227, or a variant which is at least 80% identical to SEQ ID NO: 227.

Illustrative CD28 hinge domain and CD8
transmembrane domain (SEQ ID NO: 227):
IEVMYPPPYLDNEKSNGTIIHVKGKHLCPSPLFPGPSKPIYIWAPLAGT
CGVLLLSLVIT

Suitably, the variant may be at least 85%, at least 90%, at least 95%, at least 97%, at least 98% or at least 99% identical to SEQ ID NO: 227.

Suitably, the CAR may comprise the CD28 transmembrane domain. Suitably, the transmembrane domain may comprise the amino acid sequence shown as SEQ ID NO: 228, or a variant which is at least 80% identical to SEQ ID NO: 228.

Illustrative CD28 TMdomain (AA 153 to 179) (SEQ
ID NO: 228):
FWVLVVVGGVLACYSLLVTVAFIIFWV

Suitably, the variant may be at least 85%, at least 90%, at least 95%, at least 97%, at least 98% or at least 99% identical to SEQ ID NO: 228.

It will be appreciated by a skilled person that a variant of a transmembrane domain, must still be capable of being present across or spanning the cell membrane, i.e. of being a transmembrane domain.

Endodomain

The CAR may comprise an endodomain comprising one or more intracellular signalling domains and optionally one or more co-stimulatory domains.

The CAR may comprise one or more intracellular signalling domains.

The “intracellular signalling domain” as used herein refers to the intracellular part of the CAR that participates in transducing the message of the effective CAR binding to a HLA (preferably HLA-A2) into the interior of the Treg to elicit Treg function, e.g. immunosuppressive function.

Suitable intracellular signalling domains will be apparent to those of skill in the art. The intracellular signalling domain is necessary to transduce the effector function signal and direct the Treg to perform its specialized function upon antigen binding. Examples of intracellular signalling domains include, but are not limited to, ζ chain of the T-cell receptor or any of its homologs (e.g., η chain, FcεR1γ and β chains, MB1 (Iga) chain, B29 (Igβ) chain, etc.), CD3 polypeptides (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28 or their signalling domains. The intracellular signalling domain may be human CD3 zeta signalling domain, FcγRIII, FcsRI, cytoplasmic tails of Fc receptors, immunoreceptor tyrosine-based activation motif (ITAM) bearing cytoplasmic receptors or combinations thereof.

Most preferably, the intracellular signalling domain may comprise the intracellular signalling domain of human CD3 zeta signalling domain. Suitably, the intracellular signalling domain may comprise the amino acid sequence shown as SEQ ID NO: 229, or a variant which is at least 80% identical to SEQ ID NO: 229.

Illustrative CD3 zeta signalling domain (SEQ ID
NO: 229):
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

Suitably, the variant may be at least 85%, at least 90%, at least 95%, at least 97%, at least 98% or at least 99% identical to SEQ ID NO: 229.

The intracellular signalling domain of the CAR may comprise the CD28 signalling domain. Suitably, the intracellular signalling domain may comprise the amino acid sequence shown as SEQ ID NO: 230, or a variant which is at least 80% identical to SEQ ID NO: 230.

Illustrative CD28 signalling domain (SEQ ID
NO: 230):
RSKRSRLLHSDYMNMTPRRPGPTRKHYQPYAPPRDFAAYRS

Suitably, the variant may be at least 85%, at least 90%, at least 95%, at least 97%, at least 98% or at least 99% identical to SEQ ID NO: 230.

The intracellular signalling domain of the CAR may comprise the CD27 signalling domain. Suitably, the intracellular signalling domain may comprise the amino acid sequence shown as SEQ ID NO: 231, or a variant which is at least 80% identical to SEQ ID NO: 231.

Illustrative CD27 signalling domain (SEQ ID
NO: 231):
QRRKYRSNKGESPVEPAEPCHYSCPREEEGSTIPIQEDYRKPEPACSP

In one embodiment, the intracellular signalling domain comprises a signalling motif which has at least 85%, at least 90%, at least 95%, at least 97%, at least 98% or at least 99% identity to SEQ ID NO: 231.

Additional intracellular signalling domains will be apparent to those of skill in the art and may be used in connection with alternate embodiments of the invention.

The CAR may also comprise one or more co-stimulatory domains.

A “co-stimulatory domain” as used herein refers to an intracellular part of the CAR that may promote Treg function (e.g. immunosuppressive function), expansion, and/or persistence.

Accordingly, the CAR may comprise a compound endodomain comprising a fusion of the one or more co-stimulatory domains to that of an intracellular signalling domain e.g. CD3. Such a compound endodomain may be referred to as a second generation CAR which can transmit an activating and co-stimulatory signal simultaneously after antigen recognition. The co-stimulatory domain most commonly used is that of CD28. This supplies the most potent co-stimulatory signal—namely immunological signal 2, which triggers Treg proliferation. Suitable co-stimulatory domains will be apparent to those of skill in the art.

Accordingly, the CAR preferably comprises a CD28 co-stimulatory domain. Suitably, the one or more co-stimulatory domains may comprise the amino acid sequence shown as SEQ ID NO: 230 or a variant which is at least 80% (e.g. at least 85%, at least 90%, at least 95%, at least 97%, at least 98% or at least 99%) identical to SEQ ID NO: 230.

Suitably, the one or more co-stimulatory domains may comprise the amino acid sequence shown as SEQ ID NO: 231, or a variant which is at least 80% (e.g. at least 85%, at least 90%, at least 95%, at least 97%, at least 98% or at least 99%) identical to SEQ ID NO: 231.

Suitably, the one or more co-stimulatory domains may comprise one or more TNF receptor family signalling domain, such as the signalling domain of OX40, 4-1 BB, ICOS or TNFRSF25.

Illustrative sequences for OX40, 4-1 BB, ICOS and TNFRSF25 signalling domains are shown below. The one or more co-stimulatory domains may comprise one or more of SEQ ID NOs: 232-235 or a variant which is at least 80% identical to one or more of SEQ ID NOs: 232-235.

Illustrative OX40 signalling domain (SEQ ID
NO: 232):
ALYLLRRDQRLPPDAHKPPGGGSFRTPIQEEQADAHSTLAKI
Illustrative 41BB signalling domain (SEQ ID
NO: 233):
KRGRKKLLYIFKQPFMRPVQTTQEEDGCSCRFPEEEEGGCEL
Illustrative ICOS signalling domain (SEQ ID
NO: 234):
CWLTKKKYSSSVHDPNGEYMFMRAVNTAKKSRLTDVTL
Illustrative TNFRSF25 signalling domain (SEQ ID
NO: 235):
TYTYRHCWPHKPLVTADEAGMEALTPPPATHLSPLDSAHTLLAPPDSSE
KICTVQLVGNSWTPGYPETQEALCPQVTWSWDQLPSRALGPAAAPTLSP
ESPAGSPAMMLQPGPQLYDVMDAVPARRWKEFVRTLGLREAEIEAVEVE
IGRFRDQQYEMLKRWRQQQPAGLGAVYAALERMGLDGCVEDLRSRLQRG
P

The one or more co-stimulatory domains may comprise a variant of one or more of OX40, 4-1 BB, ICOS and TNFRSF25 signalling domains which has at least 85%, at least 90%, at least 95%, at least 97%, at least 98% or at least 99% identity to any one of SEQ ID NOs: 232-235.

The endodomain of the CAR may comprise the CD28 signalling domain and the CD3 zeta signalling domain. Suitably, the endodomain may comprise the amino acid sequence shown as SEQ ID NO: 236, or a variant which is at least 80% identical to SEQ ID NO: 236.

Illustrative CD28 signalling domain and CD3 zeta
signalling domain (SEQ ID NO: 236):
RSKRSRLLHSDYMNMTPRRPGPTRKHYQPYAPPRDFAAYRSRVKFSRSA
DAPAYQQGQNQLYNELNLGRREEYDVLDKRRGRDPEMGGKPRRKNPQEG
LYNELQKDKMAEAYSEIGMKGERRRGKGHDGLYQGLSTATKDTYDALHM
QALPPR

The endodomain may comprise a variant which is at least 85%, at least 90%, at least 95%, at least 97%, at least 98% or at least 99% identical to SEQ ID NO: 236.

A variant of an intracellular signalling domain and/or of a costimulatory domain may have the same or similar function to the comparative wildtype intracellular signalling domain and/or costimulatory domain, e.g. may have at least 40, 50, 60, 70, 80, 90, 95, 100, 110, 120, 130, 140 or 150% of the function of the wildtype domain (e.g. of the signalling ability of the wildtype domain).

Other Domains

In some embodiments the CAR comprises one or more signal peptides.

The CAR may comprise a leader sequence which targets it to the endoplasmic reticulum pathway for expression on the cell surface. An illustrative leader sequence is a CD8 leader sequence. Illustrative leader sequences are shown below as SEQ ID NO: 237 and SEQ ID NO: 241.

Illustrative CD8 leader (SEQ ID NO: 237):
MALPVTALLLPLALLLHAARP
Illustrative leader sequence (SEQ ID NO: 241):
MALPVTALLLPLALLLHAAAP

The leader sequence may comprise or consist of a variant which is at least 80%, at least 85%, at least 90%, at least 95%, at least 97%, at least 98% or at least 99% identical to SEQ ID NO: 237 or SEQ ID NO: 241.

In some embodiments the CAR comprise one or more reporter domains, optionally in combination with a self-cleaving or cleavage domain.

Suitable reporter domains are well known in the art and include, but are not limited to, fluorescent proteins—such as GFP. The use of a reporter domain is advantageous as it allows a Treg in which a polynucleotide or vector of the present invention has been successfully introduced (such that the encoded CAR is expressed) to be selected and isolated from a starting cell population using common methods, e.g. flow cytometry. Suitably, the reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding the CAR and the reporter domain may be separated by a co-expression site which enables expression of each polypeptide as a discrete entity. Suitable co-expression sites are known in the art and include, for example, internal ribosome entry sites (IRES) and self-cleaving peptides. Suitable self-cleaving or cleavage domains include, but are not limited to P2A peptide, T2A peptide, E2A peptide, F2A peptide, and furin site.

Illustrative CAR Constructs

Illustrative CAR constructs for use in the present invention are shown below. The CAR may comprise a sequence which has at least 80% (e.g. at least 85%, at least 90%, at least 95%, at least 97%, at least 98%, at least 99% or 100%) identity to one of SEQ ID NOs: 209 or 210. Preferably any such variant has at least partial functionality as compared to SEQ ID NO: 209 or 210. For example, the variant may have at least 10%, at least 20%, at least 30%, at least 40%, at least 50%, at least 60%, at least 70%, at least 80%, or at least 90% of the function of an amino acid sequence shown as one of SEQ ID NO: 209 or 210. The variant may have functionality similar to or the same level as one of SEQ ID NO: 209 or 210 or may have functionality of a greater level than an amino acid sequence shown as one of SEQ ID NO: 209 or 210 (e.g. increased by at least 10%, at least 20%, at least 30%, at least 40% or at least 50%).

CD8 hinge-CD8 TM domain-CD28 signalling
domain-CD3 zeta signalling domain (SEQ ID
NO: 209):
TTTPAPRPPTPAPTIASQPLSLRPEACRPAAGGAVHTRGLDFACDI
YIWAPLAGTCGVLLLSLVITRSKRSRLLHSDYMNMTPRRPGPTRKH
YQPYAPPRDFAAYRSRVKFSRSADAPAYQQGQNQLYNELNLGRREE
YDVLDKRRGRDPEMGGKPRRKNPQEGLYNELQKDKMAEAYSEIGMK
GERRRGKGHDGLYQGLSTATKDTYDALHMQALPPR
CD28 hinge-CD8 TM domain-CD28 signalling
domain-CD3 zeta signalling domain (SEQ ID
NO: 210):
IEVMYPPPYLDNEKSNGTIIHVKGKHLCPSPLFPGPSKPIYIWAPL
AGTCGVLLLSLVITRSKRSRLLHSDYMNMTPRRPGPTRKHYQPYAP
PRDFAAYRSRVKFSRSADAPAYQQGQNQLYNELNLGRREEYDVLDK
RRGRDPEMGGKPRRKNPQEGLYNELQKDKMAEAYSEIGMKGERRRG
KGHDGLYQGLSTATKDTYDALHMQALPPR

Particularly, SEQ ID NO. 209 or a variant thereof may be used in conjunction with an antigen binding domain comprising: (i) SEQ ID NOs: 8-10 and SEQ ID NOs: 20-22 or derivatives thereof; (ii) SEQ ID NOs: 11-13 and SEQ ID NOs: 23-25 or derivatives thereof; or (iii) SEQ ID NOs: 14-16 and SEQ ID NOs: 26-28, or derivatives thereof.

A vector of the invention may particularly comprise a CAR comprising the domains as shown in the below table.

| Antigen binding domain | Hinge and TM | | Intracellular |
|---|---|---|---|
| comprising CDR SEQ ID Nos: | domains | Costimulatory domain | signalling domain |
| SEQ ID Nos 8-10 and 20-22; | CD8 | CD28 | CD3zeta |
| SEQ ID Nos 11-13 and 23-25; or | | | |
| SEQ ID Nos 14-16 and 26-28 | | | |
| SEQ ID Nos 8-10 and 20-22; | CD8 | CD28; and | CD3zeta |
| SEQ ID Nos 11-13 and 23-25; or | | OX40 or 41BB | |
| SEQ ID Nos 14-16 and 26-28 | | | |
| SEQ ID Nos 8-10 and 20-22; | CD28 | CD28 | CD3zeta |
| SEQ ID Nos 11-13 and 23-25; or | | | |
| SEQ ID Nos 14-16 and 26-28 | | | |
| SEQ ID Nos 8-10 and 20-22; | CD28 | CD28; and | CD3zeta |
| SEQ ID Nos 11-13 and 23-25; or | | OX40 or 41BB | |
| SEQ ID Nos 14-16 and 26-28 | | | |

Illustrative polynucleotide sequences encoding SEQ ID NO: 209 and SEQ ID NO: 210 are shown below. The polynucleotide encoding a HLA-A-specific CAR may comprise a sequence which has at least 70% (e.g. at least 75%, at least 80%, at least 85%, at least 90%, at least 95%, at least 97%, at least 98%, at least 99% or 100%) identity to SEQ ID NO: 211 or SEQ ID NO: 212. Preferably any such variant encodes a HLA-specific CAR that has at least partial functionality as compared to SEQ ID NOs: 209 or 210. For example, the HLA-specific CAR encoded by the variant may have at least 10%, at least 20%, at least 30%, at least 40%, at least 50%, at least 60%, at least 70%, at least 80%, or at least 90% of the function of an amino acid sequence shown as SEQ ID NO: 209 or 210. The HLA-specific CAR encoded by the variant may have functionality similar to or the same level as one of SEQ ID NO: 209 or 210 or may have functionality of a greater level than an amino acid sequence shown as one of SEQ ID NO: 209 or 210 (e.g. increased by at least 10%, at least 20%, at least 30%, at least 40% or at least 50%).

Illustrative polynucleotide encoding SEQ ID NO: 209
(SEQ ID NO: 211)
ACCACCACCCCCGCCCCCCGCCCCCCCACCCCCGCCCCCACCATCGCCAG
CCAGCCCCTGAGCCTGCGCCCCGAGGCCTGCCGCCCCGCCGCCGGCGGCG
CCGTGCACACCCGCGGCCTGGACTTCGCCTGCGACATCTACATCTGGGCC
CCCCTGGCCGGCACCTGCGGCGTGCTGCTGCTGAGCCTGGTGATCACCCG
CAGCAAGCGCAGCCGCCTGCTGCACAGCGACTACATGAACATGACCCCCC
GCCGCCCCGGCCCCACCCGCAAGCACTACCAGCCCTACGCCCCCCCCCGC
GACTTCGCCGCCTACCGCAGCCGCGTGAAGTTCAGCCGCAGCGCCGACGC
CCCCGCCTACCAGCAGGGCCAGAACCAGCTGTACAACGAGCTGAACCTGG
GCCGCCGCGAGGAGTACGACGTGCTGGACAAGCGCCGCGGCCGCGACCCC
GAGATGGGCGGCAAGCCCCGCCGCAAGAACCCCCAGGAGGGCCTGTACAA
CGAGCTGCAGAAGGACAAGATGGCCGAGGCCTACAGCGAGATCGGCATGA
AGGGCGAGCGCCGCCGCGGCAAGGGCCACGACGGCCTGTACCAGGGCCTG
AGCACCGCCACCAAGGACACCTACGACGCCCTGCACATGCAGGCCCTGCC
CCCCCGC
Illustrative polynucleotide encoding SEQ ID NO: 210
(SEQ ID NO: 212)
ATCGAGGTGATGTACCCCCCCCCCTACCTGGACAACGAGAAGAGCAACGG
CACCATCATCCACGTGAAGGGCAAGCACCTGTGCCCCAGCCCCCTGTTCC
CCGGCCCCAGCAAGCCCATCTACATCTGGGCCCCCCTGGCCGGCACCTGC
GGCGTGCTGCTGCTGAGCCTGGTGATCACCCGCAGCAAGCGCAGCCGCCT
GCTGCACAGCGACTACATGAACATGACCCCCCGCCGCCCCGGCCCCACCC
GCAAGCACTACCAGCCCTACGCCCCCCCCCGCGACTTCGCCGCCTACCGC
AGCCGCGTGAAGTTCAGCCGCAGCGCCGACGCCCCCGCCTACCAGCAGGG
CCAGAACCAGCTGTACAACGAGCTGAACCTGGGCCGCCGCGAGGAGTACG
ACGTGCTGGACAAGCGCCGCGGCCGCGACCCCGAGATGGGCGGCAAGCCC
CGCCGCAAGAACCCCCAGGAGGGCCTGTACAACGAGCTGCAGAAGGACAA
GATGGCCGAGGCCTACAGCGAGATCGGCATGAAGGGCGAGCGCCGCCGCG
GCAAGGGCCACGACGGCCTGTACCAGGGCCTGAGCACCGCCACCAAGGAC
ACCTACGACGCCCTGCACATGCAGGCCCTGCCCCCCCGC

Relative Position of the First and Second Polynucleotides

In preferred embodiments the first polynucleotide encoding FOXP3 is upstream of the second polynucleotide encoding a HLA specific CAR. Accordingly, in preferred embodiments the first polynucleotide and the second polynucleotide are operably linked to the same promoter and the first polynucleotide is upstream of the second polynucleotide

As used herein the term “upstream” when referring to polynucleotides means that the “upstream” polynucleotide is 5′ to the “downstream” polynucleotide. In other words, in preferred embodiments the vector may have the orientation of: 5′ FOXP3-HLA specific CAR 3′. In more preferred embodiment the vector may have the structure: 5′ Promoter-FOXP3-HLA specific CAR 3′. Here, FOXP3 expression is directly driven by the promoter for optimal expression.

Importantly, a configuration in which FOXP3 precedes the CAR in the 5′ to 3′ direction ensures that the CAR expression can only occur when (exogenous) FOXP3 has been expressed and that expression of CAR without FOXP3 does not occur. This is a particular advantage in the present context of an engineered Treg, as it reduces the risk of an engineered Treg acquiring an effector phenotype and/or reduces the risk associated with introducing the CAR into a T effector cell present in a starting population.

Cleavage Site and Internal Ribosome Entry Sites

The polynucleotide encoding FOXP3 may be separated from the polynucleotide encoding the HLA specific CAR by a nucleic acid sequence which enables both the nucleic acid sequence encoding FOXP3 and the nucleic acid sequence encoding the CAR to be expressed from the same mRNA transcript.

For example, the vector may comprise an internal ribosome entry site (IRES) between the nucleic acid sequences which encode (i) FOXP3 and (ii) the HLA specific CAR. An IRES is a nucleotide sequence that allows for translation initiation in the middle of a mRNA sequence. Suitably, the vector may have the structure: 5′ Promoter-FOXP3-IRES-HLA specific CAR 3′.

Suitably, the vector may comprise a nucleic acid sequence encoding (i) FOXP3 and (ii) the HLA specific CAR linked by a cleavage domain. Such sequences may either auto-cleave during protein production or may be cleaved by common enzymes present in the cell. preferably, the cleavage domain auto-cleaves. Accordingly, inclusion of a cleavage domain in the polypeptide sequence enables a first and a second polypeptide to be expressed as a single polypeptide, which is subsequently cleaved to provide discrete, separated functional polypeptides. Suitably, the vector may have the structure: 5′ Promoter-FOXP3-cleavage domain-HLA specific CAR 3′. Suitable cleavage domains may include the furin site (e.g. SEQ ID NOs: 238 or 239).

Furin site-cleavage domain:
(SEQ ID NO: 238)
RXXR (preferentially: RRKR (SEQ ID NO: 239))

Preferably, the vector comprises a nucleic acid sequence encoding (i) FOXP3 and (ii) the HLA specific CAR linked by a self-cleaving sequence. Such sequences auto-cleave during protein production. Suitably, the vector may have the structure: 5′ Promoter-FOXP3-self-cleaving sequence-HLA specific CAR 3′.

Preferably, the self-cleaving sequence is a polynucleotide sequence encoding a 2A self-cleaving peptide. Suitably, the vector may have the structure: 5′ Promoter-FOXP3-2A self-cleaving peptide-HLA specific CAR 3′.

Overall, 2A peptides lead to relatively high levels of downstream protein expression compared to other strategies for multi-gene co-expression, and they are small in size thus bearing a lower risk of interfering with the function of co-expressed genes (Liu, Z., et al., 2017. Scientific reports, 7(1), p. 2193).

Moreover, the mechanism of 2A-mediated “self-cleavage” is ribosome skipping the formation of a glycyl-prolyl peptide bond at the C-terminus of the 2A. A highly conserved sequence GDVEXNPGP (SEQ ID NO: 240) is shared by different 2As at the C-terminus, and is essential for the creation of steric hindrance and ribosome skipping. There are three possibilities for a 2A-mediated skipping event: (1) successful skipping and recommencement of translation results in two “cleaved” proteins: the protein upstream of the 2A is attached to the complete 2A peptide except for the C-terminal proline, and the protein downstream of the 2A is attached to one proline at the N-terminus; (2) successful skipping but ribosome fall-off and discontinued translation results in only the protein upstream of 2A; and (3) unsuccessful skipping and continued translation resulting in a fusion protein. Due to the risk of possibility (2), a configuration in which FOXP3 precedes the CAR in the 5′ to 3′ direction ensures that the CAR expression can only occur when FOXP3 has been expressed and that expression of CAR without FOXP3 does not occur.

Suitable self-cleaving peptides include a P2A peptide, a T2A peptide, a E2A peptide, and a F2A peptide.

Suitably, the vector may have the structure:
(i) 5′ Promoter-FOXP3-P2A-HLA specific CAR 3′;
(ii) 5′ Promoter-FOXP3-T2A-HLA specific CAR 3′;
(iii) 5′ Promoter-FOXP3-E2A-HLA specific CAR 3′; or
(iv) 5′ Promoter-FOXP3-F2A-HLA specific CAR 3′.

Preferably, the vector may have the structure: 5′ Promoter-FOXP3-P2A-HLA specific CAR 3′.

Illustrative sequences for a P2A peptide, a T2A peptide, a E2A peptide, and a F2A peptide are shown below. The self-cleaving sequence may comprise or consist of a polynucleotide sequence encoding any of SEQ ID NOs: 213, 215, 217, 219, 242, 244, 246, or 248 or encoding a variant which is at least 80% identical to any of SEQ ID NOs: 213, 215, 217, 219, 242, 244, 246, or 248.

Illustrative P2A peptide-cleavage domain
(SEQ ID NO: 213):
GSGATNFSLLKQAGDVEENPGP
Illustrative T2A peptide-cleavage domain
(SEQ ID NO: 215):
GSGEGRGSLLTCGDVEENPGP
Illustrative E2A peptide-cleavage domain
(SEQ ID NO: 217):
GSGQCTNYALLKLAGDVESNPGP
Illustrative F2A peptide-cleavage domain
(SEQ ID NO: 219):
GSGVKQTLNFDLLKLAGDVESNPGP
Illustrative P2A peptide-cleavage domain
(SEQ ID NO: 242):
ATNFSLLKQAGDVEENPGP
Illustrative T2A peptide-cleavage domain
(SEQ ID NO: 244):
EGRGSLLTCGDVEENPGP
Illustrative E2A peptide-cleavage domain
(SEQ ID NO: 246):
QCTNYALLKLAGDVESNPGP
Illustrative F2A peptide-cleavage domain
(SEQ ID NO: 248):
VKQTLN FDLLKLAG DVESN PG P

The self-cleaving sequence may comprise or consist of a polynucleotide sequence encoding a variant which has at least 85%, at least 90%, at least 95%, at least 97%, at least 98% or at least 99% identity to any one of SEQ ID NOs: 213, 215, 217, 219, 242, 244, 246, or 248.

Illustrative polynucleotide sequences encoding a P2A peptide, a T2A peptide, a E2A peptide, and a F2A peptide are shown below. The self-cleaving sequence may comprise or consist of a polynucleotide selected from any of SEQ ID NOs: 214, 216, 218, 220, 243, 245, 247, or 249 or a variant which is at least 80% identical to any of SEQ ID NOs: 214, 216, 218, 220, 243, 245, 247, or 249.

Illustrative P2A peptide-cleavage domain
(SEQ ID NO: 214):
GGCAGCGGCGCCACCAACTTCAGCCTGCTGAAGCAGGCCGGCGACGTGG
AGGAGAACCCCGGCCCC
Illustrative T2A peptide-cleavage domain
(SEQ ID NO: 216):
GGCAGCGGCGAGGGCCGCGGCAGCCTGCTGACCTGCGGCGACGTGGAGG
AGAACCCCGGCCCC
Illustrative E2A peptide-cleavage domain
(SEQ ID NO: 218);
GGCAGCGGCCAGTGCACCAACTACGCCCTGCTGAAGCTGGCCGGCGACG
TGGAGAGCAACCCCGGCCCC
Illustrative F2A peptide-cleavage domain
(SEQ ID NO: 220):
GGCAGCGGCGTGAAGCAGACCCTGAACTTCGACCTGCTGAAGCTGGCCG
GCGACGTGGAGAGCAACCCCGGCCCC
Illustrative P2A peptide-cleavage domain
(SEQ ID NO: 243):
GCCACCAACTTCAGCCTGCTGAAGCAGGCCGGCGACGTGGAGGAGAACC
CCGGCCCC
Illustrative T2A peptide-cleavage domain
(SEQ ID NO: 245):
GAGGGCCGCGGCAGCCTGCTGACCTGCGGCGACGTGGAGGAGAACCCCG
GCCCC
Illustrative E2A peptide-cleavage domain
(SEQ ID NO: 247):
CAGTGCACCAACTACGCCCTGCTGAAGCTGGCCGGCGACGTGGAGAGCA
ACCCCGGCCCC
Illustrative F2A peptide-cleavage domain
(SEQ ID NO: 249):
GTGAAGCAGACCCTGAACTTCGACCTGCTGAAGCTGGCCGGCGACGTGG
AGAGCAACCCCGGCCCC

The self-cleaving sequence may comprise or consist of a variant which has at least 85%, at least 90%, at least 95%, at least 97%, at least 98% or at least 99% identity to any one of SEQ ID NOs: 214, 216, 218, 220, 243, 245, 247, or 249.

Polynucleotides and Polypeptides

In the present invention, HLA specific cells are generated by introducing into the cells a polynucleotide (sometimes referred to herein as a first polynucleotide) encoding a FOXP3 polypeptide and a polynucleotide (sometimes referred to herein as a second polynucleotide) encoding a HLA specific chimeric antigen receptor (CAR).

The terms “polynucleotide” and “nucleic acid” are intended to be synonymous with each other. A polynucleotide may be any suitable type of nucleotide sequence, such as a synthetic RNA/DNA sequence, a cDNA sequence or a partial genomic DNA sequence.

The term “polypeptide” is synonymous with “protein” and means a series of residues, typically L-amino acids, connected to one another typically by peptide bonds between the α-amino and carboxyl groups of adjacent amino acids.

Numerous different polynucleotides can encode the same polypeptide as a result of the degeneracy of the genetic code. The skilled person may make nucleotide substitutions that do not affect the polypeptide sequence encoded by the polynucleotides to reflect the codon usage of any particular host organism in which the polypeptides are to be expressed.

The polynucleotide may comprise DNA or RNA, may be single-stranded or double-stranded and may include synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. Polynucleotides may be modified by any method in the art. Such modifications may enhance the in vivo activity or life span of the polynucleotide.

The polynucleotide may be in isolated or recombinant form. It may be incorporated into a vector and the vector may be incorporated into a host cell.

The polynucleotide may be codon optimised. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. Suitably, the polynucleotide may be codon optimised for expression in a murine model of disease. Suitably, the polynucleotide may be codon optimised for expression in a human subject.

Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimisation may also involve the removal of mRNA instability motifs and cryptic splice sites.

Variants, Derivatives and Fragments

In addition to the specific polypeptides and polynucleotides mentioned herein, the present invention also encompasses the use of derivatives, variants and fragments thereof.

The term “derivative” as used herein, in relation to proteins or polypeptides of the present invention includes any substitution of, variation of, modification of, replacement of, deletion of and/or addition of one (or more) amino acid residues from or to the sequence providing that the resultant polypeptide retains the desired function (for example, where the derivative or variant is an antigen binding domain, the desired function may be the ability of the antigen binding domain to bind its target antigen, or where the derivative or variant is a signalling domain, the desired function may be the ability of that domain to signal (e.g. activate or inactivate a downstream molecule). The variant or derivative may have at least 10%, at least 20%, at least 30%, at least 40%, at least 50%, at least 60%, at least 70%, at least 80%, or at least 90% function as compared to the corresponding reference sequence; a similar or the same level of function as compared to the corresponding reference sequence; or an increased level of function as compared to the corresponding reference sequence, for example function increased by at least 10%, at least 20%, at least 30%, at least 40%, or at least 50% compared to an unmodified sequence.

Typically, amino acid substitutions may be made, for example from 1, 2 or 3 to 10 or 20 substitutions provided that the modified sequence retains the required activity or ability e.g. at least 10%, at least 20%, at least 30%, at least 40%, at least 50%, at least 60%, at least 70%, at least 80%, or at least 90% activity as compared to the corresponding reference sequence; a similar or the same level of activity as compared to an the corresponding reference sequence; or an increased level of activity as compared to the corresponding reference sequence, for example activity increased by at least 10%, at least 20%, at least 30%, at least 40%, or at least 50% compared to an unmodified sequence. Amino acid substitutions may include the use of non-naturally occurring analogues.

Proteins or peptides used in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

The derivative may be a homolog or variant. The term “homologue” or “variant” as used herein means an entity having a certain homology with the wild type amino acid sequence and the wild type nucleotide sequence. The term “homology” can be equated with “identity”.

A homologous or variant sequence may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to the subject sequence. Typically, the homologues will have similar chemical properties/functions e.g. comprise the same binding sites etc. as the subject amino acid sequence or the amino acid sequence encoded by the subject nucleotide sequence. Although homology can also be considered in terms of similarity (i.e. amino acid residues having similar chemical properties/functions), in the context of the present invention it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence.

Once the software has produced an optimal alignment, it is possible to calculate percentage homology, preferably percentage sequence identity. The software typically does this as part of the sequence comparison and generates a numerical result.

“Fragments” typically refers to a selected region of the polypeptide or polynucleotide that is of interest functionally. “Fragment” thus refers to an amino acid or nucleic acid sequence that is a portion of a full-length polypeptide or polynucleotide, respectively.

Such derivatives, variants and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Vectors

In some embodiments of the invention the polynucleotide (sometimes referred to herein as a first polynucleotide) encoding FOXP3 and/or the polynucleotide (sometimes referred to herein as a second polynucleotide) encoding a HLA specific CAR are a contiguous portion of a vector.

In preferred embodiments the first polynucleotide encoding FOXP3 and the second polynucleotide encoding a HLA specific CAR are present in a single vector.

A vector is a tool that allows or facilitates the transfer of an entity from one environment to another. In accordance with the present invention, and by way of example, some vectors used in recombinant nucleic acid techniques allow entities, such as a segment of nucleic acid (e.g. a heterologous DNA segment, such as a heterologous cDNA segment), to be transferred into a target cell.

Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest. Preferably, the vector is capable of sustained high-level expression in host cells.

Suitably, the vectors used in the present invention may be, for example, plasmid, mRNA or viral vectors. In preferred embodiments, the vector is a viral vector.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

The vector may include a promoter for the expression of the polynucleotide, and optionally one or more regulators of the promoter. In preferred embodiments the first polynucleotide encoding FOXP3 and the second polynucleotide encoding a HLA specific CAR are present in a single vector and the first polynucleotide and the second polynucleotide are operably linked to the same promoter (e.g. a LTR).

Vectors of the invention may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation. Multiple vectors could be used for transduction/transfection.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a polynucleotide of the invention.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14: 556) and combinations thereof.

Viral Vectors

The vector of the present invention may be a viral vector.

The viral vector may be any viral vector known to those of skill in the art. In particular, a number of viral based systems have been developed for gene transfer into mammalian cells.

Suitably the viral vector is a retroviral vector, a lentiviral vector, an adenoviral vector, a pox viral vector, or a vaccinia virus vector. Preferably, the viral vector is a retroviral vector (e.g. a gamma retroviral vector) or a lentiviral vector. More preferably, the viral vector is a lentiviral vector.

Suitably, the vector used in the present invention is a retrovirus-based vector which has been genetically engineered so that it cannot replicate and produce progeny infectious virus particles once the virus has entered the target cell. There are many retroviruses that are widely used for delivery of genes both in tissue culture conditions and in living organisms. Examples include and are not limited to murine leukemia virus (MLV), human immunodeficiency virus (HIV-1), equine infectious anaemia virus (EIAV), mouse mammary tumour virus (MMTV), Rous sarcoma virus (RSV), Fujinami sarcoma virus (FuSV), Moloney murine leukemia virus (Mo-MLV), FBR murine osteosarcoma virus (FBR MSV), Moloney murine sarcoma virus (Mo-MSV), Abelson murine leukemia virus (A-MLV), Avian myelocytomatosis virus-29 (MC29), and Avian erythroblastosis virus (AEV) and all other retroviridiae including lentiviruses. A detailed list of retroviruses may be found in Coffin et al., 1997, “retroviruses”, Cold Spring Harbour Laboratory Press Eds: J M Coffin, S M Hughes, H E Varmus pp 758-763.

The basic structure of a retrovirus genome is a 5′ LTR and a 3′ LTR, between or within which are located a packaging signal to enable the genome to be packaged, a primer binding site, integration sites to enable integration into a host cell genome and gag, pol and env genes encoding the packaging components—these are polypeptides required for the assembly of viral particles. More complex retroviruses have additional features, such as rev and RRE sequences in HIV, which enable the efficient export of RNA transcripts of the integrated provirus from the nucleus to the cytoplasm of an infected target cell.

In the provirus, these genes are flanked at both ends by regions called long terminal repeats (LTRs). The LTRs are responsible for proviral integration, and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are identical sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

In a retroviral vector genome of the present invention gag, pol and env may be absent or not functional. The R regions at both ends of the RNA are repeated sequences. U5 and U3 represent unique sequences at the 5′ and 3′ ends of the RNA genome respectively.

Preferably the envelope is one which allows transduction of human cells, preferably T cells, most preferably Tregs. Examples of suitable env genes include, but are not limited to, VSV-G, a MLV amphotropic env such as the 4070A env, the RD114 feline leukaemia virus env or haemagglutinin (HA) from an influenza virus. The Env protein may be one which is capable of binding to a receptor on a limited number of human cell types and may be an engineered envelope containing targeting moieties. The env and gag-pol coding sequences are transcribed from a promoter and optionally an enhancer active in the chosen packaging cell line and the transcription unit is terminated by a polyadenylation signal. For example, if the packaging cell is a human cell, a suitable promoter-enhancer combination is that from the human cytomegalovirus major immediate early (hCMV-MIE) gene and a polyadenylation signal from SV40 virus may be used. Other suitable promoters and polyadenylation signals are known in the art.

In preferred embodiments, the vector of the present invention is a lentiviral vector. Lentivirus vectors are part of a larger group of retroviral vectors. A detailed list of lentiviruses may be found in Coffin et al (“Retroviruses” 1997 Cold Spring Harbour Laboratory Press Eds: J M Coffin, S M Hughes, H E Varmus pp 758-763). In brief, lentiviruses can be divided into primate and non-primate groups. Examples of primate lentiviruses include but are not limited to: the human immunodeficiency virus (HIV), the causative agent of human acquired-immunodeficiency syndrome (AIDS), and the simian immunodeficiency virus (SIV). The non-primate lentiviral group includes the prototype “slow virus” visna/maedi virus (VMV), as well as the related caprine arthritis-encephalitis virus (CAEV), equine infectious anaemia virus (EIAV) and the more recently described feline immunodeficiency virus (FIV) and bovine immunodeficiency virus (BIV).

A distinction between the lentivirus family and other types of retroviruses is that lentiviruses have the capability to infect both dividing and non-dividing cells. In contrast, other retroviruses are unable to infect non-dividing or slowly dividing cells such as those that make up, for example, muscle, brain, lung and liver tissue. As lentiviruses are able to transduce terminally differentiated/primary cells, the use of a lentiviral screening strategy allows library selection in a primary target non-dividing or slowly dividing host cell.

The vector of the present invention may be packaged into a viral particle. Methods of packaging viral particles are well known to those of skill in the art. For example, methods of producing and packaging retroviral vectors are described in Merten, O. W., 2004. The Journal of Gene Medicine: A cross-disciplinary journal for research on the science of gene transfer and its clinical applications, 6(S1), pp. S105-S124. For example, methods of producing and packaging lentiviral particles are described in in Merten, O. W., et al., 2016. Molecular Therapy-Methods & Clinical Development, 3, p. 16017 and Zufferey, R., 2002. Production of lentiviral vectors. In Lentiviral Vectors (pp. 107-121). Springer, Berlin, Heidelberg.

Illustrative Vector Constructs

Illustrative vectors for use in the present invention are described below.

Suitably, the vector may comprise (5′ to 3′):
(i) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 3, or a functional fragment thereof, a cleavage domain and/or an IRES, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211;
(ii) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 3, or a functional fragment thereof, a cleavage domain and/or an IRES, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 212;
(iii) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 4, or a functional fragment thereof, a cleavage domain and/or an IRES, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211; or
(iv) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 4, or a functional fragment thereof, a cleavage domain and/or an IRES, and a second polynucleotide which comprises a polynucleotide sequence which has at least 70% (e.g. at least 75%, at least 80%, at least 85%, at least 90%, at least 95%, at least 98%, at least 99%, or 100% identity) identity to SEQ ID NO: 212.

The first polynucleotide and the second polynucleotide may be operably linked to the same promoter. Suitably the vector is a viral vector, preferably a retroviral vector or a lentiviral vector.

Suitably, the vector may comprise (5′ to 3′):
(i) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 3, or a functional fragment thereof, a self-cleaving sequence which has at least 70% (e.g. at least 75%, at least 80%, at least 85%, at least 90%, at least 95%, at least 98%, at least 99%, or 100% identity) identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211;
(ii) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 3, or a functional fragment thereof, a self-cleaving sequence which has at least 70% (e.g. at least 75%, at least 80%, at least 85%, at least 90%, at least 95%, at least 98%, at least 99%, or 100% identity) identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 212;
(iii) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 4, a self-cleaving sequence which has at least 70% (e.g. at least 75%, at least 80%, at least 85%, at least 90%, at least 95%, at least 98%, at least 99%, or 100% identity) identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211; or
(iv) a first polynucleotide which comprises or consists of a polynucleotide sequence which has at least 70% identity (e.g. at least 75%, at least 80%, at least 85%, at least 90%, at least 95%, at least 98%, at least 99%, or 100% identity) to SEQ ID NO: 4, or a functional fragment thereof, a self-cleaving sequence which has at least 70% (e.g. at least 75%, at least 80%, at least 85%, at least 90%, at least 95%, at least 98%, at least 99%, or 100% identity) identity to SEQ ID NO: 214, and a second polynucleotide which comprises a polynucleotide sequence which has at least 70% (e.g. at least 75%, at least 80%, at least 85%, at least 90%, at least 95%, at least 98%, at least 99%, or 100% identity) identity to SEQ ID NO: 212.

The first polynucleotide and the second polynucleotide may be operably linked to the same promoter. Suitably the vector is a viral vector, preferably a retroviral vector or a lentiviral vector.

Suitably, the vector may comprise (5′ to 3′):
(i) a first polynucleotide which comprises or consists of a polynucleotide sequence which has at least 95% identity to SEQ ID NO: 3, or a functional fragment thereof, a self-cleaving sequence which has at least 95% identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211;
(ii) a first polynucleotide which comprises or consists of a polynucleotide sequence which has at least 95% identity to SEQ ID NO: 3, or a functional fragment thereof, a self-cleaving sequence which has at least 95% identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 212;
(iii) a first polynucleotide which comprises or consists of a polynucleotide sequence which has at least 95% identity to SEQ ID NO: 4, a self-cleaving sequence which has at least 95% identity to SEQ ID NO: 214, and a second polynucleotide which comprises a polynucleotide sequence which has at least 70% identity (e.g. at least 75%, at least 80%, at least 85%, at least 90%, at least 95%, at least 98%, at least 99%, or 100% identity) to SEQ ID NO: 211; or
(iv) a first polynucleotide which comprises or consists of a polynucleotide sequence which has at least 95% identity to SEQ ID NO: 4, or a functional fragment thereof, a self-cleaving sequence which has at least 95% identity to SEQ ID NO: 214, and a second polynucleotide which comprises a polynucleotide sequence which has at least 70% (e.g. at least 75%, at least 80%, at least 85%, at least 90%, at least 95%, at least 98%, at least 99%, or 100% identity) identity to SEQ ID NO: 212.

The first polynucleotide and the second polynucleotide may be operably linked to the same promoter. Suitably the vector is a viral vector, preferably a retroviral vector or a lentiviral vector.

Regulators of Gene Expression

The vector of the invention may include a promoter for the expression of the polynucleotide(s). When the first polynucleotide encoding FOXP3 and the second polynucleotide encoding a HLA specific CAR are present in the same vector, the first polynucleotide and the second polynucleotide may be operably linked to the same promoter.

A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person.

In one embodiment, the promoter may be a LTR, for example the LTR of the vector (e.g. a retroviral LTR or a lentiviral LTR).

Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals for gene expression are found in LTRs: enhancer, promoter (can have both transcriptional enhancers or regulatory elements), transcription initiation (such as capping), transcription terminator and polyadenylation signal.

Suitably, the vector of the invention may include a 5′LTR and a 3′LTR. When the first polynucleotide encoding FOXP3 and the second polynucleotide encoding a HLA specific CAR are present in the same vector, the first polynucleotide and the second polynucleotide may be operably linked to the same LTR.

The vector of the invention may comprise one or more additional regulatory sequences which may act pre- or post-transcriptionally. “Regulatory sequences” are any sequences which facilitate expression of the polypeptides, e.g. act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g. operably linked to the promoter. Suitably, the vector may comprise the nucleotide sequence shown as SEQ ID NO: 250, or a variant which is at least 80% identical to SEQ ID NO: 250. Suitably, the variant may be at least 85%, at least 90%, at least 95%, at least 97%, at least 98% or at least 99% identical to SEQ ID NO: 250.

Illustrative WPRE (SEQ ID NO: 250):
AATCAACCTCTGGATTACAAAATTTGTGAAAGATTGACTGGTATTCTTA
ACTATGTTGCTCCTTTTACGCTATGTGGATACGCTGCTTTAATGCCTTT
GTATCATGCTATTGCTTCCCGTATGGCTTTCATTTTCTCCTCCTTGTAT
AAATCCTGGTTGCTGTCTCTTTATGAGGAGTTGTGGCCCGTTGTCAGGC
AACGTGGCGTGGTGTGCACTGTGTTTGCTGACGCAACCCCCACTGGTTG
GGGCATTGCCACCACCTGTCAGCTCCTTTCCGGGACTTTCGCTTTCCCC
CTCCCTATTGCCACGGCGGAACTCATCGCCGCCTGCCTTGCCCGCTGCT
GGACAGGGGCTCGGCTGTTGGGCACTGACAATTCCGTGGTGTTGTCGGG
GAAATCATCGTCCTTTCCTTGGCTGCTCGCCTATGTTGCCACCTGGATT
CTGCGCGGGACGTCCTTCTGCTACGTCCCTTCGGCCCTCAATCCAGCGG
ACCTTCCTTCCCGCGGCCTGCTGCCGGCTCTGCGGCCTCTTCCGCGTCT
TCGCCTTCGCCCTCAGACGAGTCGGATCTCCCTTTGGGCCGCCTCCCCG
C

Cells

In one aspect the present invention provides a cell comprising a vector according to the present invention. Suitably, the cell is a T cell or a T cell progenitor.

A T cell is a type of lymphocyte which develops in the thymus gland and plays a central role in the immune response. T cells can be distinguished from other lymphocytes by the presence of a T-cell receptor on the cell surface. These immune cells originate as precursor cells, derived from bone marrow, and develop into several distinct types of T cells once they have migrated to the thymus gland. T cell differentiation may continue after they have left the thymus.

T cells are grouped into a series of subsets based on their function. CD4 and CD8 T cells are selected in the thymus, but undergo further differentiation in the periphery to specialized cells which have different functions. T cell subsets were initially defined by function, but also have associated gene or protein expression patterns. Conventional adaptive T cells include helper CD4+ T cells, cytotoxic CD8+ T cells, memory T cells, and regulatory CD4+ T cells. Innate-like T cells include natural killer T cells, mucosal associated invariant T cells and gamma delta T cells.

Regulatory T Cells (Tregs)

Regulatory T cells (Treg) are immune cells with suppressive function that control cytopathic immune responses and are essential for the maintenance of immunological tolerance.

In one aspect the present invention provides a Treg comprising a vector according to the present invention. In other words, the present invention provides an engineered Treg.

An “engineered Treg” as used herein means a Treg which has been modified to comprise or express a polynucleotide which is not naturally encoded by the cell, in particular a polynucleotide encoding a FOXP3 polypeptide and/or a polynucleotide encoding a HLA specific CAR as described herein. Methods for engineering Tregs are known in the art and include, but are not limited to, genetic modification of Tregs e.g. by transduction such as retroviral or lentiviral transduction, transfection (such as transient transfection, DNA or RNA based) including lipofection, polyethylene glycol, calcium phosphate and electroporation. Any suitable method may be used to introduce a nucleic acid sequence into a Treg.

As used herein, the term “Treg” refers to a T cell with immunosuppressive function.

Suitably, “immunosuppressive function” may refer to the ability of the Treg to reduce or inhibit one or more of a number of physiological and cellular effects facilitated by the immune system in response to a stimulus such as a pathogen, antigen, e.g. an alloantigen, or an autoantigen. Examples of such effects include increased proliferation of conventional T cells (Tconv) and secretion of pro-inflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs of the present invention may proliferate 5% less, 10% less, 20% less, 30% less, 40% less, 50% less, 60% less, 70% less, 90% less, 95% less or 99% less than the same Tconv cells cultured in the absence of Tregs of the invention.

Antigen-specific Tconv cells co-cultured with Tregs of the invention may express at least 10%, at least 20%, at least 30%, at least 40%, at least 50%, or at least 60% less effector cytokine than corresponding Tconv cells cultured in the absence of Tregs of the invention.

The effector cytokine may be selected from IL-2, IL-17, TNFα, GM-CSF, IFN-γ, IL-4, IL-5, IL-9, IL-10 and IL-13.

Suitably the effector cytokine may be selected from IL-2, IL-17, TNFα, GM-CSF and IFN-γ.

Suitably, the Treg is a T cell which expresses the markers CD4, CD25 and FOXP3 (CD4+CD25+FOXP3+).

Marker levels can be determined by any method known to those of skill in the art, for example flow cytometry.

Tregs may also express CTLA-4 (cytotoxic T-lymphocyte associated molecule-4) and/or GITR (glucocorticoid-induced TNF receptor). Treg cells are present in the peripheral blood, lymph nodes, and tissues.

Suitably, the Treg may be identified using the cell surface markers CD4 and CD25 in the absence of or in combination with low-level expression of the surface protein CD127 (CD4⁺CD25⁺CD127⁻ or CD4⁺CD25⁺CD127^{low} or CD4⁺CD25^{hi}CD127⁻ or CD4⁺CD25^{hi}CD127^{low}). The use of such markers to identify Tregs is known in the art and described in Liu et al. (JEM; 2006; 203; 7(10); 1701-1711), for example.

The Treg may be a CD4⁺CD25⁺FOXP3⁺ T cell or a CD4⁺CD25^{hi}FOXP3⁺ T cell.

The Treg may be a CD4⁺CD25⁺CD127⁻ T cell or a CD4⁺CD25^{hi}CD127⁻ T cell.

The Treg may be a CD4⁺CD25⁺FOXP3⁺CD127⁻ T cell or a CD4⁺CD25^{hi}FOXP3+CD127⁻ T cell.

The Treg may have a demethylated Treg-specific demethylated region (TSDR). The TSDR is an important methylation-sensitive element regulating FOXP3 expression (Polansky, J. K., et al., 2008. European journal of immunology, 38(6), pp. 1654-1663).

The Treg may be natural or thymus-derived, adaptive or peripherally-derived, or in vitro-induced (Abbas, A. K., et al., 2013. Nature immunology, 14(4), p. 307-308). Suitably, the Treg may be CD4⁺CD25⁺FOXP3⁺Helios⁺Neuropilin 1⁺. Preferably the Treg is a natural Treg.

Further suitable Tregs include, but are not limited to, Tr1 cells, CD8⁺FOXP3⁺ T cells; and γδ FOXP3⁺ T cells.

Suitably, the Treg is isolated from peripheral blood mononuclear cells (PBMCs) obtained from a subject. Suitably the subject is a mammal, preferably a human.

Suitably, the Treg is matched (e.g. HLA-matched) or is autologous to a subject to whom the engineered Treg is to be administered. Suitably, the subject to whom the engineered Treg is to be administered is a mammal, preferably a human. The Treg may be generated ex vivo either from a patient's own peripheral blood (1st party), or in the setting of a haematopoietic stem cell transplant from donor peripheral blood (2nd party), or peripheral blood from an unconnected donor (3rd party). Suitably the Treg is autologous to the subject to whom the engineered Treg is to be administered.

In a preferred embodiment, the Treg is isolated from peripheral blood mononuclear cells (PBMCs) obtained from a subject and is matched (e.g. HLA-matched) or is autologous to the subject to whom the engineered Treg is to be administered.

Suitably, the Treg is part of a population of Tregs. Suitably, the population of Tregs comprises at least 70% Tregs, such as at least 75%, at least 85%, at least 90%, at least 95%, at least 97%, at least 98% or at least 99% Tregs. Such a population may be referred to as an “enriched Treg population” or an “enriched Treg sample”.

As used herein, the term “conventional T cell” or Icon means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. An engineered Treg may be generated from a Icon by in vitro culture of CD4⁺CD25⁻FOXP3⁻ cells in the presence of IL-2 and TGF-β.

The Treg of the present invention may be derived from a stem cell. In particular, the Treg of the present invention may be derived from a stem cell in vitro. The Treg may be derived from ex-vivo differentiation of inducible progenitor cells or embryonic progenitor cells to the Treg. A polynucleotide or vector of the invention may introduced into the inducible progenitor cells or embryonic progenitor cells prior to, or after, differentiation to a Treg.

As used herein, the term “stem cell” means an undifferentiated cell which is capable of indefinitely giving rise to more stem cells of the same type, and from which other, specialised cells may arise by differentiation. Stem cells are multipotent. Stem cells may be for example, embryonic stem cells or adult stem cells.

As used herein, the term “progenitor cell” means a cell which is able to differentiate to form one or more types of cells but has limited self-renewal in vitro.

Suitably, the cell is capable of being differentiated into a T cell, such as a Treg.

Suitably, the cell may be an embryonic stem cell (ESC). Suitably, the cell is a haematopoietic stem cell or haematopoietic progenitor cell. Suitably, the cell is an induced pluripotent stem cell (iPSC). Suitably, the cell may be obtained from umbilical cord blood. Suitably, the cell may be obtained from adult peripheral blood.

In some aspects, hematopoietic stem and progenitor cell (HSPCs) may be obtained from umbilical cord blood. Cord blood can be harvested according to techniques known in the art (e.g., U.S. Pat. Nos. 7,147,626 and 7,131,958 which are incorporated herein by reference).

In one aspect, HSPCs may be obtained from pluripotent stem cell sources, e.g., induced pluripotent stem cells (iPSCs) and embryonic stem cells (ESCs).

As used herein, the term “hematopoietic stem and progenitor cell” or “HSPC” refers to a cell which expresses the antigenic marker CD34 (CD34+) and populations of such cells. In particular embodiments, the term “HSPC” refers to a cell identified by the presence of the antigenic marker CD34 (CD34+) and the absence of lineage (lin) markers. The population of cells comprising CD34+ and/or Lin(−) cells includes haematopoietic stem cells and hematopoietic progenitor cells.

HSPCs can be obtained or isolated from bone marrow of adults, which includes femurs, hip, ribs, sternum, and other bones. Bone marrow aspirates containing HSPCs can be obtained or isolated directly from the hip using a needle and syringe. Other sources of HSPCs include umbilical cord blood, placental blood, mobilized peripheral blood, Wharton's jelly, placenta, fetal blood, fetal liver, or fetal spleen. In particular embodiments, harvesting a sufficient quantity of HSPCs for use in therapeutic applications may require mobilizing the stem and progenitor cells in the subject.

As used herein, the term “induced pluripotent stem cell” or “iPSC” refers to a non-pluripotent cell that has been reprogrammed to a pluripotent state. Once the cells of a subject have been reprogrammed to a pluripotent state, the cells can then be programmed to a desired cell type, such as a hematopoietic stem or progenitor cell (HSC and HPC respectively).

As used herein, the term “reprogramming” refers to a method of increasing the potency of a cell to a less differentiated state.

As used herein, the term “programming” refers to a method of decreasing the potency of a cell or differentiating the cell to a more differentiated state.

The invention also provides an engineered Treg having higher FOXP3 expression than a non-engineered Treg and an engineered Treg having higher FOXP3 expression than a corresponding, non-engineered Treg.

“Higher FOXP3 expression” means levels of FOXP3 mRNA or protein in the engineered Treg are higher than they were before the Treg was manipulated by human intervention to alter its gene expression.

The “higher FOXP3 expression” may be defined and determined as described herein.

Suitably, the level of FOXP3 mRNA and/or protein in an engineered Treg according to the present invention (or a population of such Tregs) may be increased to at least 1.5-fold greater, 2-fold greater, or 5-fold greater than the level in a corresponding non-engineered Treg (or population of such Tregs).

Suitably, the level of CD25 mRNA and/or protein in an engineered Treg according to the present invention (or a population of such Tregs) may be increased to at least 1.5-fold greater, 2-fold greater, or 5-fold greater than the level in a corresponding non-engineered Treg (or population of such Tregs).

Suitably, the level of CTLA-4 mRNA and/or protein in an engineered Treg according to the present invention (or a population of such Tregs) may be increased to at least 1.5-fold greater, 2-fold greater, or 5-fold greater than the level in a corresponding non-engineered Treg (or population of such Tregs).

The engineered Treg of the present invention may comprise an exogenous polynucleotide encoding a FOXP3 polypeptide. An “exogenous polynucleotide” is a polynucleotide that originates outside the Treg.

T Effector Cells

The present invention may reduce the risk of generating an engineered T effector cell e.g. during generation of engineered Tregs.

T effector cells are relatively short-lived activated cells that defend the body in an immune response. T effector cells include cytotoxic T cells and helper T cells, which carry out cell-mediated responses. Accordingly, T effector cells may be cytotoxic T cells or helper T cells. T effector cells may express low levels of FOXP3, such that they are FOXP3^{low} w or FOXP3−. Preferably, T effector cells have no immunosuppressive function.

Most cytotoxic T cells express a subset of surface markers such as CD8, CD45 and CD54. Suitably, a cytotoxic T cell may be a CD8⁺FOXP3^{low} cell or a CD8⁺FOXP3⁻ cell.

Suitably, a cytotoxic T cell may be a CD8⁺CD45⁺FOXP3^{low} cell or a CD8⁺CD45⁺FOXP3⁻ cell.

Suitably, a cytotoxic T cell may be a CD8⁺CD54⁺FOXP3^{low} cell or a CD8⁺CD54⁺FOXP3⁻ cell.

Suitably, a cytotoxic T cell may be a CD8⁺CD45⁺CD54⁺FOXP3^{low} cell or a CD8⁺CD45⁺CD54⁺FOXP3⁻ cell.

Helper T cells (also known as T helper cells, Th cells or CD4+ cells), are a type of T cell that play an important role in the immune system, particularly in the adaptive immune system. They help the activity of other immune cells by releasing T cell cytokines. They are essential in B cell antibody class switching, in the activation and growth of cytotoxic T cells, and in maximizing bactericidal activity of phagocytes such as macrophages. T helper subtypes include Th1, Th2, Th9, Th17, Th22 and Tfh cells. Preferably, a helper T cell is a CD4+ cell without immunosuppressive function. Suitably, a helper T cell may be a CD4⁺FOXP3^{low} cell or a CD4⁺FOXP3⁻ cell.

Method of Making a Cell

Engineered Tregs of the present invention may be generated by introducing a polynucleotide (sometimes referred to herein as a first polynucleotide) encoding a FOXP3 polypeptide and/or a polynucleotide (sometimes referred to herein as a second polynucleotide) encoding a HLA specific CAR as described herein.

The term “introduce” refers to methods for inserting foreign DNA into a cell, including both transfection and transduction methods. Transfection is the process of introducing nucleic acids into a cell by non-viral methods. Transduction is the process of introducing foreign DNA into a cell via a viral vector.

The engineered Treg of the invention may be made by introducing to a Treg (e.g. by transduction or transfection) the polynucleotide(s) or vector as defined herein.

Suitably, the Treg may be from a sample isolated from a subject. The Treg may be further separated from the sample by any suitable method, for example magnetic separation.

The engineered Treg of the present invention may be generated by a method comprising the following steps:
(i) isolation of a cell-containing sample (e.g. from a subject) or provision of a cell-containing sample; and
(ii) transduction or transfection of the cell-containing sample with a polynucleotide encoding a FOXP3 polypeptide and/or a polynucleotide encoding a HLA specific CAR as described herein, or a vector as described herein (e.g. a vector encoding 5′ FOXP3-HLA specific CAR 3′), to provide a population of engineered cells.

Suitably the cell-containing sample comprises or consists of PBMCs.

Suitably, a Treg-enriched sample may be isolated from, enriched, and/or generated from the cell-containing sample prior to and/or after step (ii) of the method. For example, isolation, enrichment and/or generation of Tregs may be performed prior to and/or after step (ii) to isolate, enrich or generate a Treg-enriched sample. Isolation and/or enrichment may be performed after step (ii) to enrich for cells and/or Tregs comprising the CAR, the polynucleotide(s), and/or the vector of the present invention.

A Treg-enriched sample may be isolated or enriched by any method known to those of skill in the art, for example by FACS and/or magnetic bead sorting.

Suitably, the cell is a Treg as defined herein.

Suitably, the engineered Treg of the present invention may be generated by a method comprising the following steps:
(i) isolation of a Treg-enriched sample (e.g. from a subject) or provision of a Treg-enriched sample; and
(ii) transduction or transfection of the Treg-enriched sample with a first polynucleotide encoding a FOXP3 polypeptide and/or a second polynucleotide encoding a HLA specific CAR as described herein, or a vector as described herein (e.g. a vector encoding 5′ FOXP3-HLA specific CAR 3′), to provide a population of engineered Treg cells according to the present invention.

The cells and/or Tregs may be activated and/or expanded prior to, or after, the introduction of the polynucleotide(s) or vector, for example by treatment with an anti-CD3 monoclonal antibody or both anti-CD3 and anti-CD28 monoclonal antibodies.

The Tregs may also be expanded in the presence of anti-CD3 and anti-CD28 monoclonal antibodies in combination with IL-2. Suitably, IL-2 may be substituted with IL-15. Other components which may be used in a Treg expansion protocol include, but are not limited to rapamycin, all-trans retinoic acid (ATRA) and TGFβ.

As used herein “activated” means that a cell or population of cells has been stimulated, causing the cell(s) to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The Tregs may be washed after each step of the method, in particular after expansion.

The population of engineered Tregs may be further enriched by any method known to those of skill in the art, for example by FACS and/or magnetic bead sorting.

The steps of the method of production may be performed in a closed and sterile cell culture system.

Enhancing Engineered Treg Immunosuppression

Introducing a polynucleotide encoding a FOXP3 polypeptide (e.g. in a vector according to the present invention) into a Treg may increase FOXP3 expression and therefore enhance the ability of the Treg to suppress an immune response.

Accordingly, the present invention provides a polynucleotide encoding a FOXP3 polypeptide as described herein for use in enhancing the ability of an engineered HLA-specific Treg to suppress an immune response. Preferably, the engineered HLA-specific Treg is an engineered HLA-A2-specific Treg.

The present invention provides use of a polynucleotide encoding a FOXP3 polypeptide as described herein for enhancing the ability of an engineered HLA-specific Treg to suppress an immune response. Preferably, the engineered HLA-specific Treg is an engineered HLA-A2-specific Treg.

The present invention provides a first polynucleotide encoding a FOXP3 polypeptide as described herein and a second polynucleotide encoding a chimeric antigen receptor (CAR) as described herein for use in enhancing the ability of an engineered HLA-specific Treg to suppress an immune response, preferably an immune response against a cell expressing the HLA. The CAR may comprise a single chain antibody (scFv) antigen recognition domain as described herein, which specifically binds to a human leukocyte antigen (HLA). The first polynucleotide and the second polynucleotide may be operably linked to the same promoter. The first polynucleotide may be upstream of the second polynucleotide. The scFv antigen recognition domain may specifically bind to HLA-A2.

The present invention provides a vector as described herein, for use in enhancing the ability of an engineered HLA-specific Treg to suppress an immune response. Preferably, the engineered HLA-specific Treg is an engineered HLA-A2-specific Treg.

The present invention provides a vector as described herein for use in enhancing the ability of an engineered HLA-specific Treg to suppress an immune response, wherein the vector comprises a first polynucleotide encoding a FOXP3 polypeptide as described herein and a second polynucleotide encoding a chimeric antigen receptor (CAR) as described herein, wherein the CAR comprises a single chain antibody (scFv) antigen recognition domain which specifically binds to a human leukocyte antigen (HLA), wherein the first polynucleotide and the second polynucleotide are operably linked to the same promoter, and wherein the first polynucleotide is upstream of the second polynucleotide. The antigen recognition domain may specifically bind to HLA-A2.

The present invention provides a vector as described herein for use in enhancing the ability of an engineered HLA-specific Treg to suppress an immune response, wherein the vector comprises a first polynucleotide encoding a FOXP3 polypeptide as described herein and a second polynucleotide encoding a chimeric antigen receptor (CAR) as described herein, wherein the CAR comprises an antigen recognition domain which specifically binds to a human leukocyte antigen (HLA), wherein the first polynucleotide and the second polynucleotide are operably linked to the same promoter, wherein the first polynucleotide is upstream of the second polynucleotide and wherein the vector further comprises a polynucleotide encoding a cleavage site as described herein between the first polynucleotide and the second polynucleotide and/or an internal ribosome entry site (IRES) as described herein between the first polynucleotide and the second polynucleotide. The antigen recognition domain may specifically bind to HLA-A2.

The present invention provides use of a vector as described herein for enhancing the ability of an engineered HLA-specific Treg to suppress an immune response. Preferably, the engineered HLA-specific Treg is an engineered HLA-A2-specific Treg.

The present invention provides a method for enhancing the ability of an engineered HLA-specific Treg to suppress an immune response comprising introducing a vector as described herein into the Treg.

The vector may comprise a first polynucleotide encoding a FOXP3 polypeptide as described herein and a second polynucleotide encoding a chimeric antigen receptor (CAR) as described herein, wherein the CAR comprises a single chain antibody (scFv) antigen recognition domain which specifically binds to a human leukocyte antigen (HLA), wherein the first polynucleotide and the second polynucleotide are operably linked to the same promoter, and wherein the first polynucleotide is upstream of the second polynucleotide. The antigen recognition domain may specifically bind to HLA-A2.

The vector may comprise a first polynucleotide encoding a FOXP3 polypeptide as described herein and a second polynucleotide encoding a chimeric antigen receptor (CAR) as described herein, wherein the CAR comprises an antigen recognition domain which specifically binds to a human leukocyte antigen (HLA), wherein the first polynucleotide and the second polynucleotide are operably linked to the same promoter, wherein the first polynucleotide is upstream of the second polynucleotide and wherein the vector further comprises a polynucleotide encoding a cleavage site as described herein between the first polynucleotide and the second polynucleotide and/or an internal ribosome entry site (IRES) as described herein between the first polynucleotide and the second polynucleotide. The antigen recognition domain may specifically bind to HLA-A2.

The present invention provides a method for enhancing the ability of an engineered Treg to suppress an immune response comprising introducing a first polynucleotide encoding a FOXP3 polypeptide as described herein and a second polynucleotide encoding a HLA-specific CAR as described herein into the Treg. Preferably, the HLA-specific CAR is a HLA-A2-specific CAR. The HLA-specific CAR may comprise a single chain antibody (scFv) antigen recognition domain.

The present invention provides a method for enhancing the ability of an engineered HLA-specific Treg to suppress an immune response comprising introducing a first polynucleotide encoding a FOXP3 polypeptide as described herein and a second polynucleotide encoding a HLA-specific CAR as described herein into a cell-containing sample, wherein:
(a) the cell-containing sample comprises or consists of Tregs; and/or
(b) the cell-containing sample comprises or consists of peripheral blood mononuclear cells (PBMCs) and Tregs are enriched from the cell-containing sample prior to or after introducing the first polynucleotide and/or second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the engineered HLA-specific Treg is an engineered HLA-A2-specific Treg and the HLA-specific CAR is a HLA-A2-specific CAR. The HLA-specific CAR may comprise a single chain antibody (scFv) antigen recognition domain.

Suitably, the first polynucleotide and/or the second polynucleotide are introduced by viral transduction, preferably retroviral or lentiviral transduction.

Preferably, the first polynucleotide and the second polynucleotide are introduced in a single vector, wherein the first polynucleotide and the second polynucleotide may be operably linked to the same promoter. The vector may be a vector according to the present invention.

In other words, the present invention provides a method for enhancing the ability of an engineered HLA-specific Treg (preferably a HLA-A2-specific Treg) to suppress an immune response comprising introducing a vector as described herein into a cell-containing sample, wherein:
(a) the cell-containing sample comprises or consists of Tregs; and/or
(b) the cell-containing sample comprises or consists of peripheral blood mononuclear cells (PBMCs) and Tregs are enriched from the cell-containing sample prior to or after introducing the vector; and/or
(c) the cell-containing sample comprises or consists of PBMCs and Tregs are generated from the cell-containing sample prior to or after introducing the vector; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the vector comprises an HLA-specific CAR. The HLA-specific CAR may be a HLA-A2-specific CAR. The HLA-specific CAR may comprise a single chain antibody (scFv) antigen recognition domain.

The expression “enhancing the ability to suppress immune responses” means to increase the suppressive effect of a Treg (or population of such Tregs) on an immune response in comparison to the suppressive effect of a corresponding Treg (or population of such Tregs) which has not been modified by introducing a first polynucleotide encoding a FOXP3 polypeptide as described herein, a second polynucleotide encoding a HLA specific CAR as described herein, and/or a vector as described herein. Preferably, the immune response is an immune response against a cell expressing HLA, more preferably HLA-A2. The increase in suppressive effect may be at least 10, 20, 30, 40, 50, 60, 70, 80 or 90% and may be measured by various means, including by measuring a decrease in the production of IL-2 by T effector cells (e.g. a decrease of at least 10, 20, 30, 40, 50, 60, 70, 80 or 90%), or an increase in the production of Treg associated cytokines, such as ID 0 (e.g. an increase of at least 10, 20, 30, 40, 50, 60, 70, 80 or 90%).

The term “immune response” refers to a number of physiological and cellular effects facilitated by the immune system in response to a stimulus such as a pathogen or an autoantigen. Examples of such effects include increased proliferation of Tconv cells and secretion of cytokines. Any such effects may be used as indicators of the strength of an immune response. A relatively weaker immune response by Tconv in the presence of modified Tregs compared to non-modified Treg would indicate a relative enhancement of the modified Tregs to suppress immune responses. For example, a relative decrease in cytokine secretion would be indicative of a weaker immune response, and thus an enhancement of the ability of Tregs to suppress immune responses.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with engineered Tregs of the present invention (i.e. having increased FOXP3 expression) may proliferate 5% less, 10% less, 15% less, 20% less, 25% less, 30% less, 35% less or 40% less than the same Tconv cells co-cultured with corresponding non-engineered Tregs (i.e. that do not have increased FOXP3 expression). Preferably, the Tconv cells are HLA-specific Tconv cells. More preferably, the Tconv cells are HLA-A2-specific Tconv cells.

Antigen-specific Tconv cells co-cultured with engineered Tregs of the present invention (i.e. having increased FOXP3 expression) may show a reduction of effector cytokine that is at least 10%, at least 20%, at least 30%, at least 40%, at least 50%, or at least 60% greater than corresponding Tconv cells co-cultured with corresponding non-engineered Tregs (i.e. that do not have increased FOXP3 expression). Preferably, the Tconv cells are HLA-specific Tconv cells. More preferably, the Tconv cells are HLA-A2-specific Tconv cells.

Antigen-specific Tconv cells co-cultured with engineered Tregs of the present invention (i.e. having increased FOXP3 expression) may produce 10% or less, 20% or less, 30% or less, 40% or less, 50% or less, or 60% or less effector cytokine than corresponding Tconv cells co-cultured with corresponding non-engineered Tregs (i.e. that do not have increased FOXP3 expression). Preferably, the Tconv cells are HLA-specific Tconv cells. More preferably, the Tconv cells are HLA-A2-specific Tconv cells.

The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Antigen-specific Tconv cells co-cultured with engineered Tregs of the present invention (i.e. having increased FOXP3 expression) may achieve suppression of IL-2 production at ½, ¼, ⅛, 1/10 or 1/20 the cell number of corresponding non-engineered Tregs (i.e. that do not have increased FOXP3 expression). Preferably, the Tconv cells are HLA-specific Tconv cells. More preferably, the Tconv cells are HLA-A2-specific Tconv cells.

Reducing the Risk of an Engineered Treg Acquiring an Effector Phenotype

Introducing a polynucleotide encoding a FOXP3 polypeptide (e.g. in a vector according to the present invention) into a Treg may increase FOXP3 expression and therefore reduce the risk of an engineered Treg acquiring an effector phenotype. Moreover, a vector according to the present invention in which FOXP3 precedes a HLA specific CAR in the 5′ to 3′ direction ensures that the HLA specific CAR expression can only occur when FOXP3 has been expressed and that expression of HLA specific CAR without FOXP3 does not occur. This may further reduce the risk of an engineered HLA-specific Treg acquiring an effector phenotype.

Accordingly, the present invention provides a polynucleotide encoding a FOXP3 polypeptide as described herein for use in reducing the risk of an engineered HLA-specific Treg acquiring an effector phenotype. Preferably, the engineered HLA-specific Treg is an engineered HLA-A2-specific Treg.

The present invention provides use of a polynucleotide encoding a FOXP3 polypeptide as described herein for reducing the risk of an engineered HLA-specific Treg acquiring an effector phenotype. Preferably, the engineered HLA-specific Treg is an engineered HLA-A2-specific Treg.

The present invention provides use of a first polynucleotide encoding a FOXP3 polypeptide as described herein and a second polynucleotide encoding a chimeric antigen receptor (CAR) as described herein for reducing the risk of an engineered HLA-specific Treg acquiring an effector phenotype. The CAR may comprise a single chain antibody (scFv) antigen recognition domain as described herein, which specifically binds to a human leukocyte antigen (HLA). The first polynucleotide and the second polynucleotide may be operably linked to the same promoter. The first polynucleotide may be upstream of the second polynucleotide. The scFv antigen recognition domain may specifically bind to HLA-A2.

The present invention provides a vector as described herein for use in reducing the risk of an engineered HLA-specific Treg acquiring an effector phenotype. Preferably, the engineered HLA-specific Treg is an engineered HLA-A2-specific Treg.

The present invention provides use of a vector as described herein for reducing the risk of an engineered HLA-specific Treg acquiring an effector phenotype. Preferably, the engineered HLA-specific Treg is an engineered HLA-A2-specific Treg.

The present invention provides a method for reducing the risk of an engineered HLA-specific Treg acquiring an effector phenotype comprising introducing a vector as described herein into the Treg.

The vector may comprise a first polynucleotide encoding a FOXP3 polypeptide as described herein and a second polynucleotide encoding a chimeric antigen receptor (CAR) as described herein, wherein the CAR comprises a single chain antibody (scFv) antigen recognition domain which specifically binds to a human leukocyte antigen (HLA), wherein the first polynucleotide and the second polynucleotide are operably linked to the same promoter, and wherein the first polynucleotide is upstream of the second polynucleotide. The antigen recognition domain may specifically bind to HLA-A2.

The vector may comprise a first polynucleotide encoding a FOXP3 polypeptide as described herein and a second polynucleotide encoding a chimeric antigen receptor (CAR) as described herein, wherein the CAR comprises an antigen recognition domain which specifically binds to a human leukocyte antigen (HLA), wherein the first polynucleotide and the second polynucleotide are operably linked to the same promoter, wherein the first polynucleotide is upstream of the second polynucleotide and wherein the vector further comprises a polynucleotide encoding a cleavage site as described herein between the first polynucleotide and the second polynucleotide and/or an internal ribosome entry site (IRES) as described herein between the first polynucleotide and the second polynucleotide. The antigen recognition domain may specifically bind to HLA-A2.

The present invention provides a method for reducing the risk of an engineered HLA-specific Treg acquiring an effector phenotype comprising introducing a first polynucleotide encoding a FOXP3 polypeptide as described herein and a second polynucleotide encoding a HLA-specific CAR as described herein into the Treg. Preferably, the engineered HLA-specific Treg is an engineered HLA-A2-specific Treg and the HLA-specific CAR is a HLA-A2-specific CAR. The HLA-specific CAR may comprise a single chain antibody (scFv) antigen recognition domain.

The present invention provides a method for reducing the risk of an engineered HLA-specific Treg acquiring an effector phenotype comprising introducing a first polynucleotide encoding a FOXP3 polypeptide as described herein and a second polynucleotide encoding a HLA-specific CAR as described herein into a cell-containing sample, wherein:
(a) the cell-containing sample comprises or consists of Tregs; and/or
(b) the cell-containing sample comprises or consists of peripheral blood mononuclear cells (PBMCs) and Tregs are enriched from the cell-containing sample prior to or after introducing the first polynucleotide and/or second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the engineered HLA-specific Treg is an engineered HLA-A2-specific Treg and the HLA-specific CAR is a HLA-A2-specific CAR. The HLA-specific CAR may comprise a single chain antibody (scFv) antigen recognition domain.

Suitably, the first polynucleotide and/or the second polynucleotide are introduced by viral transduction, preferably retroviral or lentiviral transduction.

Preferably, the first polynucleotide and the second polynucleotide are introduced in a single vector, wherein the first polynucleotide and the second polynucleotide may be operably linked to the same promoter. The vector may be a vector according to the present invention.

Accordingly, the present invention provides a method for reducing the risk of an engineered HLA-specific Treg acquiring an effector phenotype comprising introducing a vector according the present invention into the Treg. Preferably, the engineered HLA-specific Treg is an engineered HLA-A2-specific Treg.

The present invention provides a method for reducing the risk of an engineered HLA-specific Treg (e.g. a HLA-A2 specific Treg) acquiring an effector phenotype comprising introducing a vector according to the present invention into a cell-containing sample, wherein:
(a) the cell-containing sample comprises or consists of Tregs; and/or
(b) the cell-containing sample comprises or consists of PBMCs and Tregs are enriched from the cell-containing sample prior to or after introducing the vector; and/or
(c) the cell-containing sample comprises or consists of PBMCs and Tregs are generated from the cell-containing sample prior to or after introducing the vector; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the vector comprises an HLA-specific CAR. The HLA-specific CAR may be a HLA-A2-specific CAR. The HLA-specific CAR may comprise a single chain antibody (scFv) antigen recognition domain.

The expression “reducing the risk of an engineered Treg acquiring an effector phenotype” may mean to reduce the chance or rate of a Treg (or population of such Tregs) acquiring an effector phenotype in comparison to the chance or rate of a corresponding Treg (or population of such Tregs) which has not been modified by introducing a polynucleotide encoding a FOXP3 polypeptide as described herein and a polynucleotide encoding a HLA specific CAR as described herein (e.g. a HLA-A2 specific CAR), and/or a vector as described herein. Preferably, the Treg is a HLA specific Treg, more preferably a HLA-A2 specific Treg. Suitably, the chance is reduced by at least 10%, at least 20%, at least 30%, at least 40%, at least 50%, at least 60%, at least 70%, at least 80%, at least 90%, or 100%. Suitably, the rate is slowed by at least 1.5-fold, at least 2-fold, at least 5-fold, at least 10-fold, at least 20-fold, at least 50-fold, or at least 100-fold.

The expression “acquiring an effector phenotype” refers to the Treg acquiring a phenotype associated with T effector cells and/or losing a phenotype associated with Tregs.

Suitably, a T cell acquiring an effector phenotype may have reduced levels of FOXP3, CD25 and/or CTLA-4, preferably FOXP3. Methods described herein may be used for determining levels of FOXP3, CD25 and/or CTLA-4 mRNA and/or protein.

Suitably, a T cell acquiring an effector phenotype has reduced levels of FOXP3 after 1 week or more, or 2 weeks or more, or 3 weeks or more, or 4 weeks or more, or 5 weeks or more, or 6 weeks or more, or 7 weeks or more, or 8 weeks or more, preferably 7 weeks or more. Suitably, in a T cell acquiring an effector phenotype the level of FOXP3 mRNA and/or protein may be reduced by at least 1.5-fold, at least 2-fold, or at least 5-fold or greater.

Suitably, a T cell acquiring an effector phenotype has reduced levels of CD25 after 1 week or more, or 2 weeks or more, or 3 weeks or more, or 4 weeks or more, or 5 weeks or more, or 6 weeks or more, or 7 weeks or more, or 8 weeks or more, preferably 7 weeks or more. Suitably, in a T cell acquiring an effector phenotype the level of CD25 mRNA and/or protein may be reduced by at least 1.5-fold, at least 2-fold, or at least 5-fold or greater.

Suitably, a T cell acquiring an effector phenotype has reduced levels of CTLA-4 after 1 week or more, or 2 weeks or more, or 3 weeks or more, or 4 weeks or more, or 5 weeks or more, or 6 weeks or more, or 7 weeks or more, or 8 weeks or more, preferably 7 weeks or more. Suitably, in a T cell acquiring an effector phenotype the level of CTLA-4 mRNA and/or protein may be reduced by at least 1.5-fold, at least 2-fold, or at least 5-fold or greater.

Reducing the Risk of Generating an Engineered T Effector Cell

Introducing a polynucleotide encoding a FOXP3 polypeptide (e.g. in a vector according to the present invention) into a T effector cell may increase FOXP3 expression and therefore reduce the risk of generating an engineered T effector cell e.g. during generation of engineered Tregs. Moreover, a vector according to the present invention in which FOXP3 precedes a HLA specific CAR in the 5′ to 3′ direction ensures that the HLA specific CAR expression can only occur when FOXP3 has been expressed and that expression of HLA specific CAR without FOXP3 does not occur. This may further reduce the risk of generating an engineered HLA-specific T effector cell e.g. during generation of engineered Tregs.

Accordingly, the present invention provides a polynucleotide encoding a FOXP3 polypeptide as described herein for use in reducing the risk of generating an engineered T effector cell, preferably during generation of engineered Tregs, for example an engineered HLA specific T effector cell, preferably during generation of engineered HLA specific Tregs. More preferably, the engineered HLA-specific T effector cell is an engineered HLA-A2-specific T effector cell and the engineered HLA-specific Treg is an engineered HLA-A2-specific Treg.

The present invention provides use of a polynucleotide encoding a FOXP3 polypeptide as described herein for reducing the risk of generating an engineered T effector cell, preferably during generation of engineered Tregs, for example an engineered HLA specific T effector cell, preferably during generation of engineered HLA specific Tregs. More preferably, the engineered HLA-specific T effector cell is an engineered HLA-A2-specific T effector cell and the engineered HLA-specific Treg is an engineered HLA-A2-specific Treg.

The present invention provides use of a first polynucleotide encoding a FOXP3 polypeptide as described herein and a second polynucleotide encoding a chimeric antigen receptor (CAR) as described herein for reducing the risk of generating an engineered T effector cell, preferably during generation of engineered Tregs, for example an engineered HLA specific T effector cell, preferably during generation of engineered HLA specific Tregs. More preferably, the engineered HLA-specific T effector cell is an engineered HLA-A2-specific T effector cell and the engineered HLA-specific Treg is an engineered HLA-A2-specific Treg. The CAR may comprise a single chain antibody (scFv) antigen recognition domain as described herein, which specifically binds to a human leukocyte antigen (HLA). The first polynucleotide and the second polynucleotide may be operably linked to the same promoter. The first polynucleotide may be upstream of the second polynucleotide. The scFv antigen recognition domain may specifically bind to HLA-A2.

The present invention provides a vector as described herein for use in reducing the risk of generating an engineered T effector cell, preferably during generation of engineered Tregs. Preferably, the engineered T effector cell is an engineered HLA-specific T effector cell and the engineered Tregs are engineered HLA-specific Tregs. More preferably, the engineered T effector cell is an engineered HLA-A2-specific T effector cell and the engineered HLA-specific Tregs are engineered HLA-A2-specific Tregs.

The present invention provides use of a vector as described herein for reducing the risk of generating an engineered T effector cell, preferably during generation of engineered Tregs. Preferably, the engineered T effector cell is an engineered HLA-specific T effector cell and the engineered Tregs are engineered HLA-specific Tregs. More preferably, the engineered T effector cell is an engineered HLA-A2-specific T effector cell and the engineered HLA-specific Tregs are engineered HLA-A2-specific Tregs.

The present invention provides a method for reducing the risk of generating an engineered T effector cell, preferably during generation of engineered Tregs, comprising introducing a vector as described herein into the T effector cell. Preferably, the engineered T effector cell is an engineered HLA-specific T effector cell and the engineered Tregs are engineered HLA-specific Tregs. More preferably, the engineered T effector cell is an engineered HLA-A2-specific T effector cell and the engineered HLA-specific Tregs are engineered HLA-A2-specific Tregs.

The vector may comprise a first polynucleotide encoding a FOXP3 polypeptide as described herein and a second polynucleotide encoding a chimeric antigen receptor (CAR) as described herein, wherein the CAR comprises a single chain antibody (scFv) antigen recognition domain which specifically binds to a human leukocyte antigen (HLA), wherein the first polynucleotide and the second polynucleotide are operably linked to the same promoter, and wherein the first polynucleotide is upstream of the second polynucleotide. The antigen recognition domain may specifically bind to HLA-A2.

The vector may comprise a first polynucleotide encoding a FOXP3 polypeptide as described herein and a second polynucleotide encoding a chimeric antigen receptor (CAR) as described herein, wherein the CAR comprises an antigen recognition domain which specifically binds to a human leukocyte antigen (HLA), wherein the first polynucleotide and the second polynucleotide are operably linked to the same promoter, wherein the first polynucleotide is upstream of the second polynucleotide and wherein the vector further comprises a polynucleotide encoding a cleavage site as described herein between the first polynucleotide and the second polynucleotide and/or an internal ribosome entry site (IRES) as described herein between the first polynucleotide and the second polynucleotide. The antigen recognition domain may specifically bind to HLA-A2.

The present invention provides a method for reducing the risk of generating an engineered HLA-specific T effector cell, preferably during generation of engineered HLA-specific Tregs, comprising introducing a first polynucleotide encoding a FOXP3 polypeptide as described herein and a second polynucleotide encoding a HLA-specific CAR as described herein into the T effector cell. Preferably, the engineered HLA-specific T effector cell is an engineered HLA-A2-specific T effector cell, the engineered HLA-specific Tregs are engineered HLA-A2-specific Tregs, and the HLA-specific CAR is a HLA-A2 specific CAR. The HLA-specific CAR may comprise a single chain antibody (scFv) antigen recognition domain.

The present invention provides a method for reducing the risk of generating an engineered HLA-specific T effector cell, preferably during generation of engineered HLA-specific Tregs, comprising introducing a first polynucleotide encoding a FOXP3 polypeptide as described herein and a second polynucleotide encoding a HLA-specific CAR as described herein into a cell-containing sample, wherein:
(a) the cell-containing sample comprises T effector cells and/or Tregs; and/or
(b) the cell-containing sample comprises or consists of peripheral blood mononuclear cells (PBMCs) and Tregs are enriched from the cell-containing sample prior to or after introducing the first polynucleotide and/or second polynucleotide; and/or
(c) the cell-containing sample comprises or consists of PBMCs and Tregs are generated from the cell-containing sample prior to or after introducing the first polynucleotide and/or the second polynucleotide; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the engineered HLA-specific T effector cell is an engineered HLA-A2-specific T effector cell, the engineered HLA-specific Tregs are engineered HLA-A2-specific Tregs, and the HLA-specific CAR is a HLA-A2 specific CAR. The HLA-specific CAR may comprise a single chain antibody (scFv) antigen recognition domain.

Suitably, the first polynucleotide and/or the second polynucleotide are introduced by viral transduction, preferably retroviral or lentiviral transduction.

Preferably, the first polynucleotide and the second polynucleotide are introduced in a single vector, wherein the first polynucleotide and the second polynucleotide may be operably linked to the same promoter. The vector may be a vector according to the present invention.

Accordingly, the present invention provides a method for reducing the risk of generating an engineered HLA-specific T effector cell, preferably during generation of engineered HLA-specific Tregs comprising introducing a vector according the present invention into the T effector cell. Preferably, the engineered HLA-specific T effector cell is an engineered HLA-A2-specific T effector cell and the engineered HLA-specific Tregs are engineered HLA-A2-specific Tregs.

The present invention provides a method for reducing the risk of generating an engineered HLA-specific T effector cell (e.g. an engineered HLA-A2-specific T effector cell), preferably during generation of engineered HLA-specific Tregs (e.g. engineered HLA-A2-specific Tregs) comprising introducing a vector according to the present invention into a cell-containing sample, wherein:
(a) the cell-containing sample comprises or consists of T effector cells and/or Tregs; and/or
(b) the cell-containing sample comprises or consists of PBMCs and Tregs are enriched from the cell-containing sample prior to or after introducing the vector; and/or
(c) the cell-containing sample comprises or consists of PBMCs and Tregs are generated from the cell-containing sample prior to or after introducing the vector; and/or
(d) the cell-containing sample comprises or consists of pluripotent stem cells (PSCs) (e.g. induced pluripotent stem cells (iPSCs) or human embryonic stem cells (hESCs) and Tregs are differentiated from the PSCs prior to or after introducing the first polynucleotide and/or the second polynucleotide.

Preferably, the vector comprises an HLA-specific CAR. The HLA-specific CAR may be a HLA-A2-specific CAR. The HLA-specific CAR may comprise a single chain antibody (scFv) antigen recognition domain.

The expression “reducing the risk of generating an engineered T effector cell” may mean to reduce the chance or rate of generating an engineered T effector cell (e.g. a HLA-specific T effector cell or a HLA-A2-specific T effector cell) in comparison to the chance or rate of generating a corresponding engineered T effector cell (e.g. a HLA-specific T effector cell or a HLA-A2-specific T effector cell) which has not been modified by introducing a polynucleotide encoding a FOXP3 polypeptide as described herein and/or a vector as described herein. Preferably the chance or rate is reduced during the generation of engineered Tregs (e.g. a HLA-specific Treg or a HLA-A2-specific Treg). Suitably, the chance is reduced by at least 10%, at least 20%, at least 30%, at least 40%, at least 50%, at least 60%, at least 70%, at least 80%, at least 90%, or 100%. Suitably, the rate is slowed by at least 1.5-fold, at least 2-fold, at least 5-fold, at least 10-fold, at least 20-fold, at least 50-fold, or at least 100-fold.

Suitably, the number of engineered T effector cells (e.g. HLA-specific T effector cells or HLA-A2-specific T effector cells) generated during generation of engineered Tregs (e.g. HLA-specific Tregs or HLA-A2-specific Tregs) when introducing a polynucleotide encoding a FOXP3 polypeptide or a vector as described herein, may be reduced in comparison to the number of corresponding engineered T effector cells (e.g. HLA-specific T effector cells or HLA-A2-specific T effector cells) generated during generation of corresponding engineered Tregs (e.g. a HLA-specific Tregs or HLA-A2-specific Tregs) when using only a polynucleotide encoding a CAR (e.g. a HLA-specific CAR or a HLA-A2-specific CAR) and not a polynucleotide encoding FOXP3 or vector of the present invention. Suitably, the number of engineered T effector cells (e.g. HLA-specific T effector cells or HLA-A2-specific T effector cells) generated during generation of engineered Tregs (e.g. HLA-specific Tregs or HLA-A2-specific Tregs) may be reduced by at least 10%, at least 20%, at least 30%, at least 40%, at least 50%, at least 60%, at least 70%, at least 80%, at least 90%, or by 100%.

Pharmaceutical Composition

There is also provided a pharmaceutical composition comprising a cell of the invention (e.g. an engineered Treg of the invention), or a vector of the invention.

A pharmaceutical composition is a composition that comprises or consists of a therapeutically effective amount of a pharmaceutically active agent i.e. the vector and/or Treg. It preferably includes a pharmaceutically acceptable carrier, diluent or excipient (including combinations thereof).

By “pharmaceutically acceptable” is included that the formulation is sterile and pyrogen free. The carrier, diluent, and/or excipient must be “acceptable” in the sense of being compatible with the Treg or vector and not deleterious to the recipients thereof. Typically, the carriers, diluents, and excipients will be saline or infusion media which will be sterile and pyrogen free, however, other acceptable carriers, diluents, and excipients may be used.

Acceptable carriers, diluents, and excipients for therapeutic use are well known in the pharmaceutical art. The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The Tregs or pharmaceutical compositions according to the present invention may be administered in a manner appropriate for treating and/or preventing the disease described herein. The quantity and frequency of administration will be determined by such factors as the condition of the subject, and the type and severity of the subject's disease, although appropriate dosages may be determined by clinical trials. The pharmaceutical composition may be formulated accordingly.

The Treg or pharmaceutical composition as described herein can be administered parenterally, for example, intravenously, or they may be administered by infusion techniques. The Treg or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise Tregs of the invention in infusion media, for example sterile isotonic solution. The pharmaceutical composition may be enclosed in ampoules, disposable syringes or multiple dose vials made of glass or plastic.

The Treg or pharmaceutical composition may be administered in a single or in multiple doses. Particularly, the Treg or pharmaceutical composition may be administered in a single, one off dose. The pharmaceutical composition may be formulated accordingly.

The Treg or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for Tregs of the invention, doses of 5×10⁷ to 3×10⁹ cells, or 10⁸ to 2×10⁹ cells per subject may be administered.

The Treg may be appropriately modified for use in a pharmaceutical composition. For example, Tregs may be cryopreserved and thawed at an appropriate time, before being infused into a subject.

The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, IL18).

The invention further includes the use of kits comprising the Treg, polynucleotides, vector and/or pharmaceutical composition of the present invention. Preferably said kits are for use in the methods and uses as described herein, e.g., the therapeutic methods as described herein. Preferably said kits comprise instructions for use of the kit components.

Methods for Treating and/or Preventing Disease

Organ Transplant

The present invention provides a method of inducing tolerance to a transplant, which comprises the step of administering an engineered Treg or a pharmaceutical composition of the invention to a subject. Suitably, the subject is mammal, preferably human.

Inducing tolerance to a transplant reduces the level of a recipient's immune response to a donor transplant.

Accordingly, the present invention provides a method of treating and/or preventing transplant rejection, which comprises the step of administering an engineered Treg or a pharmaceutical composition of the invention to a subject.

The subject may be a transplant recipient wherein the transplant is selected from liver, kidney, heart, lung, pancreas, intestine, stomach, bone marrow, vascularized composite tissue graft and skin transplant. Suitably, the transplant is a liver transplant.

The engineered Tregs may be administered to a subject who has not yet rejected the transplant in order to prevent or reduce the likelihood of transplant rejection. The likelihood of transplant rejection may be reduced by at least 10%, at least 20%, at least 30%, at least 40%, or at least 50% compared to a subject in which the engineered Tregs are not administered, or the transplant rejection may be completely prevented.

The engineered Tregs may be administered to a subject who is not showing any symptoms of transplant rejection in order to reduce the likelihood of appearance of one symptom of transplant rejection such as pain or tenderness at the site of the transplant, flu-like symptoms, fever, weight-changes (e.g. weight gain), and fatigue. The at least one symptom may be reduced by at least 10%, at least 20%, at least 30%, at least 40%, or at least 50% compared to a subject in which the engineered Tregs are not administered, or the at least one symptom may be completely prevented.

The engineered Tregs or pharmaceutical composition may be administered to a subject with transplant rejection in order to reverse the rejection or slow down progression of the transplant rejection, or to lessen, reduce, or improve at least one symptom of transplant rejection such as pain or tenderness at the site of the transplant, flu-like symptoms, fever, weight-changes (e.g. weight gain), and fatigue. The at least one symptom may be lessened, reduced, or improved by at least 10%, at least 20%, at least 30%, at least 40%, or at least 50%, or the at least one symptom may be completely alleviated.

The subject may be a transplant recipient undergoing immunosuppression therapy. Suitably, the present invention may reduce the amount of immunosuppressive drugs that a transplant recipient requires, or may enable the discontinuation of immunosuppressive drugs.

Graft-Versus-Host Disease

The present invention provides a method of treating and/or preventing graft-versus-host disease (GvHD), which comprises the step of administering an engineered Treg or a pharmaceutical composition of the invention to a subject. Suitably, the subject is mammal, preferably human.

Suitably, the subject is a transplant recipient. The subject may be a transplant recipient wherein the transplant is selected from liver, kidney, heart, lung, pancreas, intestine, stomach, bone marrow, vascularized composite tissue graft and skin transplant. Suitably, the transplant is bone marrow transplant.

GvHD is a common complication following the receipt of transplanted tissue from a genetically different person. GvHD is commonly associated with stem cell transplants such as those that occur with bone marrow transplants. GvHD also applies to other forms of transplanted tissues such as liver transplants. White blood cells of the donor's immune system which remain within the donated tissue (the graft) recognize the recipient (the host) as foreign (non-self). The white blood cells present within the transplanted tissue then attack the recipient's body's cells, which leads to GvHD. The GvHD may be acute or chronic. In the classical sense, acute graft-versus-host-disease is characterized by selective damage to the liver, skin, mucosa, and the gastrointestinal tract.

Suitably, the HLA specific CAR may comprise an antigen binding domain which is capable of specifically binding to a HLA that is present in the recipient but not in the graft (transplant) donor.

The subject may be undergoing immunosuppression therapy.

The engineered Tregs may be administered to a subject with GvHD in order to slow down, reduce, or block the progression of the disease. The progression of the disease may be slowed down, reduced, or blocked by at least 10%, at least 20%, at least 30%, at least 40%, or at least 50% compared to a subject in which the engineered Tregs are not administered, or progression of the disease may be completely stopped.

The engineered Tregs or pharmaceutical composition may be administered to a subject with GvHD in order to lessen, reduce, or improve at least one symptom of GvHD such as skin rashes or itchy skin, jaundice, nausea, vomiting, diarrhea, abdominal cramping, dry or irritated eyes, dry mouth, shortness of breath, difficulty swallowing, weight loss, fatigue and muscle weakness or pain. The at least one symptom may be lessened, reduced, or improved by at least 10%, at least 20%, at least 30%, at least 40%, or at least 50%, or the at least one symptom may be completely alleviated.

The engineered Tregs may be administered to a subject who has not yet contracted and/or who is not showing any symptoms of GvHD in order to prevent or reduce the likelihood of contracting GvHD. The engineered Tregs reduce the likelihood or prevent at least one symptom of GvHD such as skin rashes or itchy skin, jaundice, nausea, vomiting, diarrhea, abdominal cramping, dry or irritated eyes, dry mouth, shortness of breath, difficulty swallowing, weight loss, fatigue and muscle weakness or pain. The at least one symptom may be reduced by at least 10%, at least 20%, at least 30%, at least 40%, or at least 50% compared to a subject in which the engineered Tregs are not administered, or the at least one symptom may be completely prevented.

Suitably, the therapeutic methods of the invention may comprise the step of administering an engineered Treg according to the invention, or obtainable (e.g. obtained) by a method according to the present invention.

Suitably, the present methods for treating and/or preventing a disease may comprise administering an engineered Treg according to the present invention (for example in a pharmaceutical composition as described herein) to a subject.

The method may involve the steps of:
(i) isolating a cell-containing sample or providing a cell-containing sample;
(ii) introducing polynucleotide(s) or a vector as defined herein to the cells; and
(iii) administering the cells from (ii) to a subject.

Suitably, the cell is a Treg as defined herein.

Suitably, an enriched Treg population may be isolated from and/or generated from the cell-containing sample prior to and/or after step (ii) of the method. For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide(s), and/or the vector of the present invention.

Suitably, the polynucleotide(s) or vector may be introduced by transduction and/or transfection.

Suitably, the cell may be autologous and/or allogenic.

Suitably, the engineered Treg may be administered is combination with one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4lg), and/or drugs inhibiting specific cytokines (IL-6, IL-17, TNFalpha, IL18). The engineered Treg may be administered simultaneously with or sequentially with (i.e. prior to or after) the one or more other therapeutic agents.

### EXAMPLES

The invention will now be further described by way of Examples, which are meant to serve to assist one of ordinary skill in the art in carrying out the invention and are not intended in any way to limit the scope of the invention.

### Example 1A—Isolation of Natural Tregs

CD4+ T cells were isolated using a CD4+ Positive selection kit. Cells were subsequently stained with flow cytometry antibodies CD4, CD25 and CD127 before FACS sorting using the BD ARIA. CD4+CD25hiCD127− Treg and CD4+CD25-CD127+ Tconv were collected in polypropylene tubes. Purity of cell sorting was determined by addition of FOXP3 PE antibody. Purity of CD4+CD25+CD127-FOXP3+ cells was routinely >70%.

### Example 1B—Transduction of Natural Tregs with FOXP3

On day 0 FACS sorted Treg and Tconv were activated separately for 48 hours by culturing 1:1 with anti-CD3 and anti-CD28 beads. On day 2 cells were counted and resuspended in complete RPMI (Tconv) or Texmacs media (Treg) at 1×10⁶/mL. Non-tissue culture-treated 24-well plates were pre-prepared by coating with retronectin then subsequently blocked with 2 bovine serum albumin in PBS and washed ×2 with PBS. Final concentration of IL-2 was 300 μ/ml for Tconv and 1000 μ/ml for Treg. Cells were incubated overnight at 37° C. before removing supernatant and supplementing with fresh complete media and IL-2. Media was changed on alternate days.

Tconv cells were grown in RPMI-1640 (Gibco) supplemented with 10% heat inactivated foetal bovine serum; 100 Units/mL penicillin; 100 μg/mL streptomycin; 2 mM L-glutamine. Regulatory T cells were cultured in Texmacs media (Miltenyi) supplemented with 100 Units/mL penicillin; 100 μg/mL streptomycin.

Flow cytometric analysis was performed at day 7-10 to assess level of transduction through expression of murine TCR constant regions and FOXP3.

### Example 1C—Proliferation and IL-2 Production of Stimulated Tconv Cells in the Presence of FOXP3-Transduced Natural Tregs

On day 10 Chinese Hamster Ovary (CHO) cells transduced with human HLA-DR4 and CD80 or CD86 were loaded with (10 μM/ml) of MBP111-129 (LSRFSWGAEGQRPGFGYGG). Suspensions were incubated for 2 hours at standard tissue culture conditions before being irradiated, washed and re-suspended at appropriate concentration.

Transduced responder T cells were stained with CFSE cell trace dye in warmed PBS at 37° C. for 3 minutes before addition of equal volumes of warm FBS and a further 3 minute incubation. Cells were washed in 5× volume of complete RPMI media before counting and resuspension at 1×10⁶ transduced cells/ml. Regulatory T cells were removed from culture, washed and re-suspended at 1×10⁶ transduced cells/ml in complete RPMI. Cells were plated 1 Treg: 0.1 CHO cells: varying ratios of Tconv for 4 days.

On day 4 cells were stained with a viability dye and analysed by flow cytometry. Percentage proliferation was determined by gating on ‘live’ cells and then the population of cells which had lower CFSE fluorescence relative to cells that were cultured without peptide.

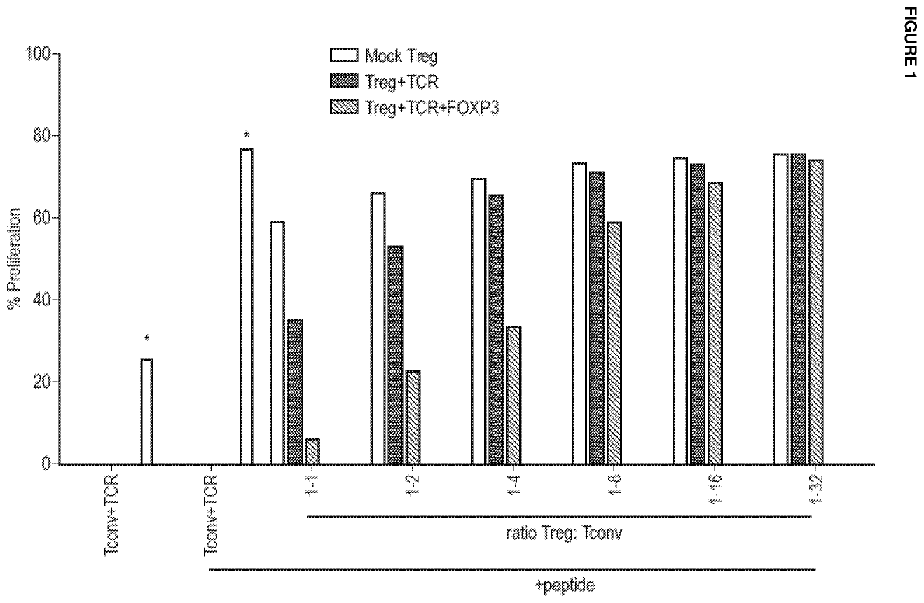

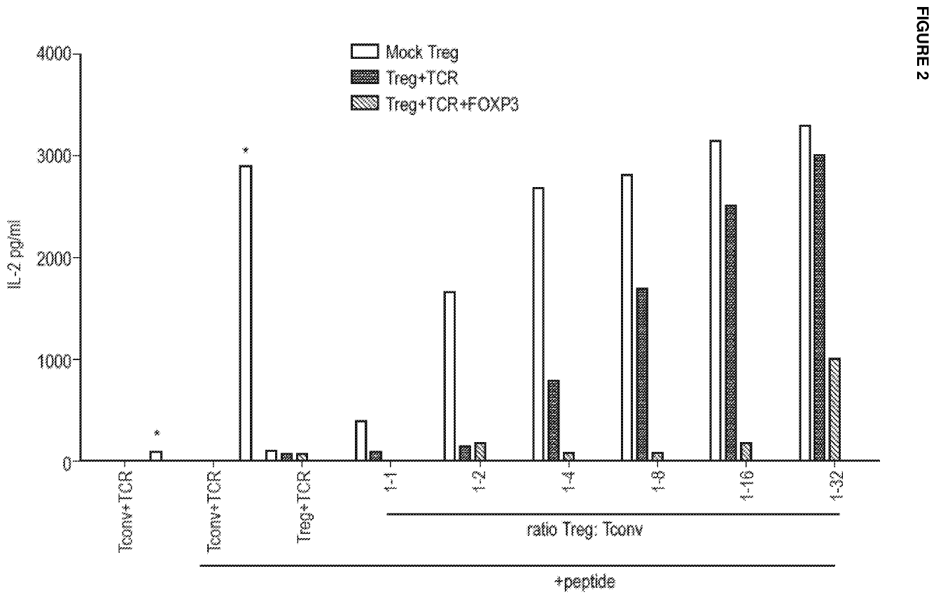

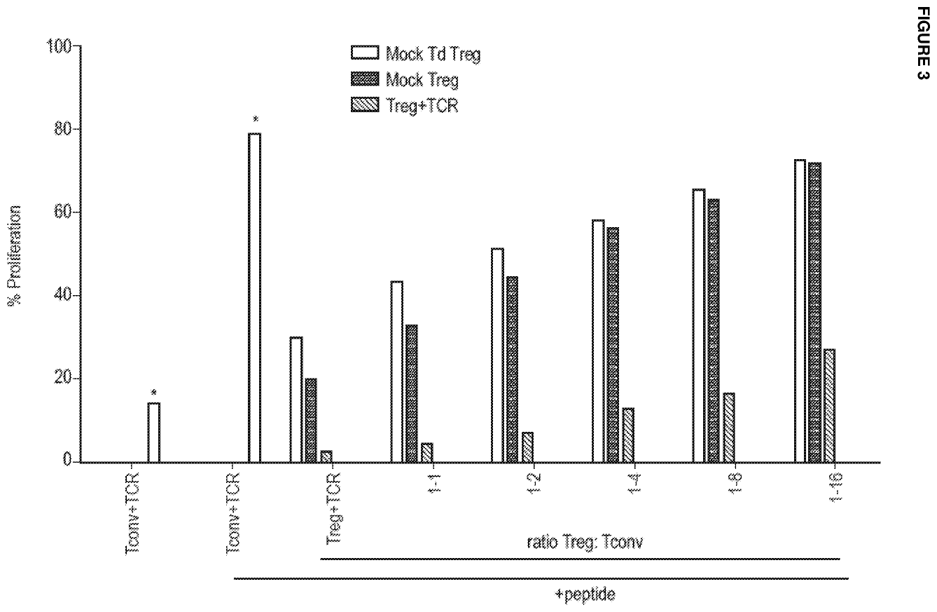

FIG. 1 shows the proliferation of TCR transduced Tconv cells with and without peptide (blue bars) and the proliferation of the same cells in the presence of Mock Treg (white bars), TCR transduced Treg or TCR+FOXP3 transduced Treg.

On day 4 supernatant was collected and assayed for IL-2 production by ELISA.

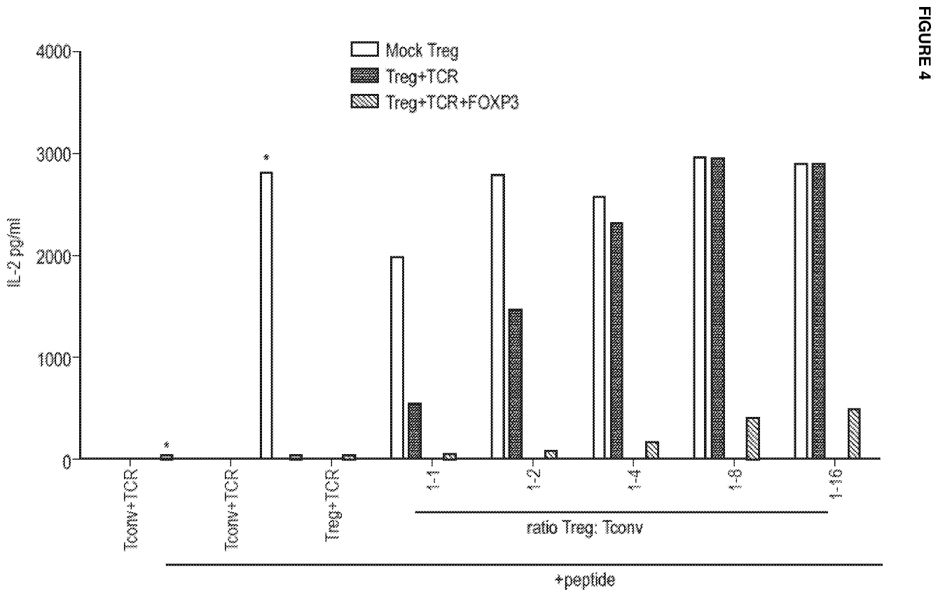

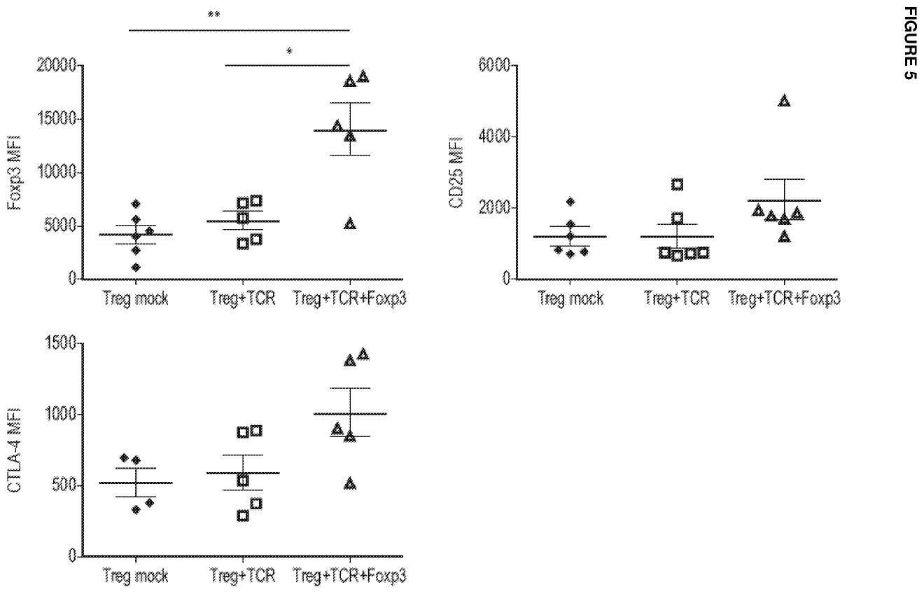

FIG. 2 shows the IL-2 production of TCR transduced Tconv cells with and without peptide (blue bars) and the proliferation of the same cells in the presence of Mock Treg (white bars), TCR transduced Treg or TCR+FOXP3 transduced Treg.

### Example 2—T Cells from a Different Donor

The experiment described in Example 1 was repeated using T cells from a different donor.

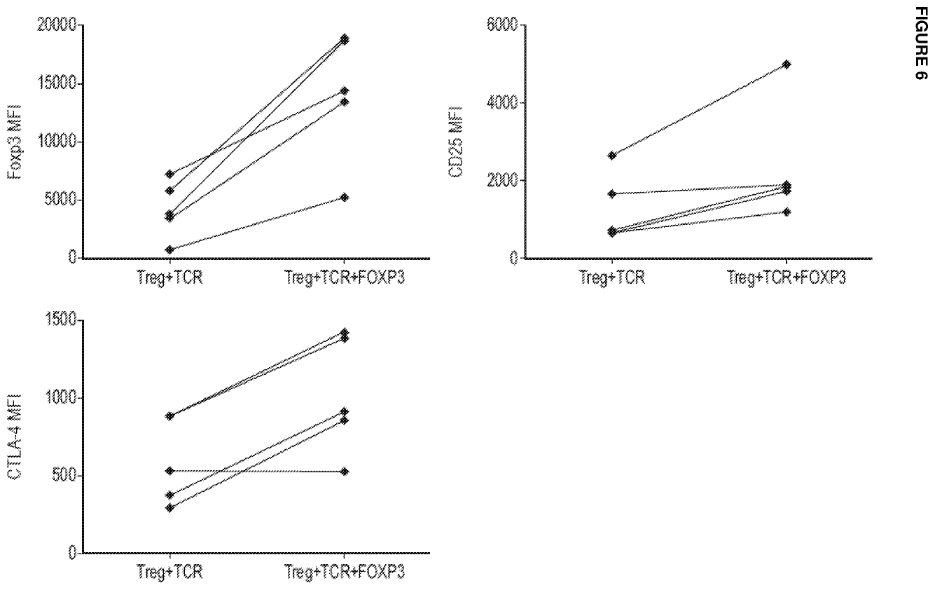

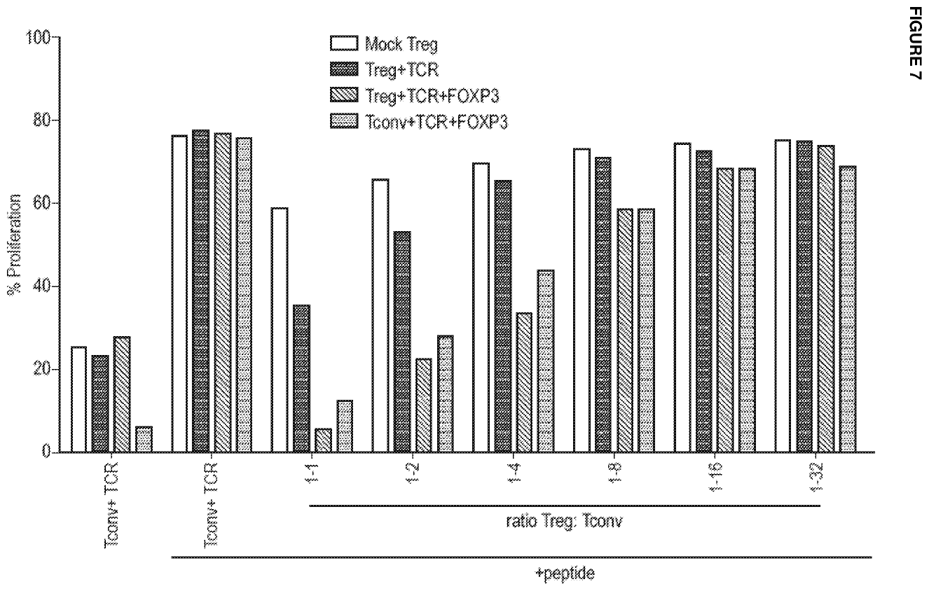

FIG. 3 shows percentage proliferation of TCR transduced T cells.

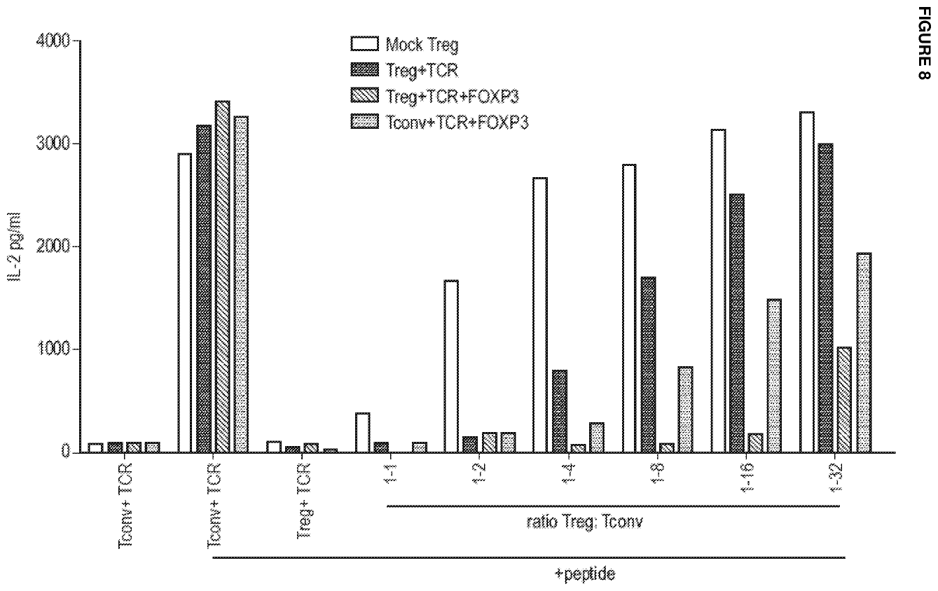

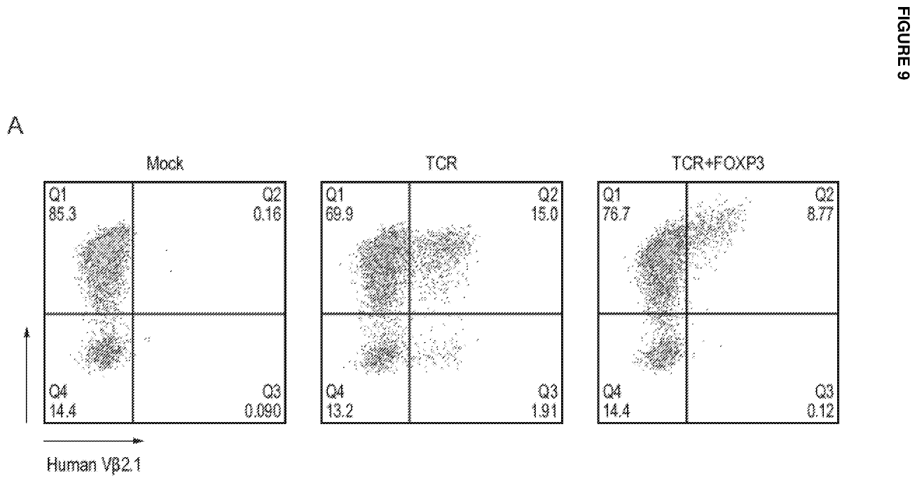

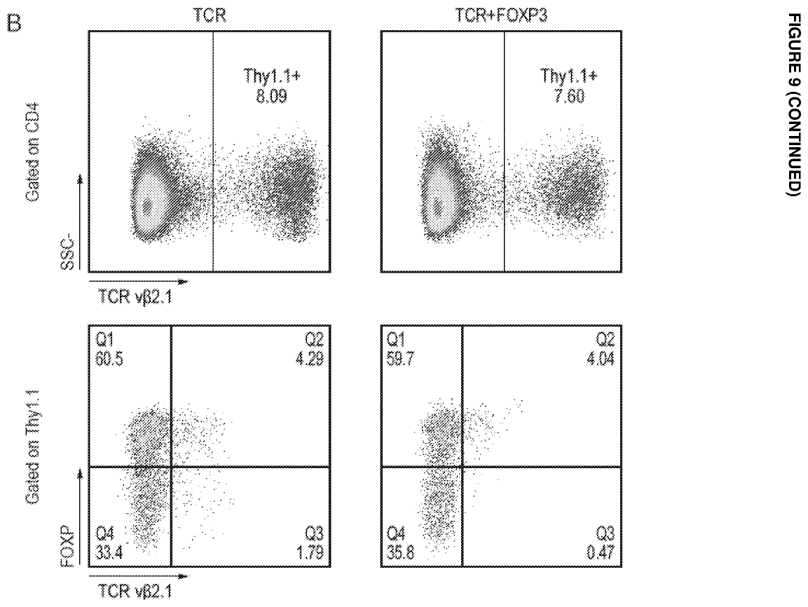

FIG. 4 shows the concentration of IL-2 in supernatants collected from co-culture experiments.

### Example 3—Expression of Treg Markers in Transduced Natural Tregs

Mock-transduced Tregs or Tregs transduced with TCR or TCR+FOXP3 were analysed by flow cytometry for the expression of Treg markers (FOXP3, CD25 and CTLA-4) at day 7-10.

FIG. 5 shows the mean fluorescence intensity (MFI) of each marker. Dots are representative of individual experiments. 1-way ANOVA was used for statistical analysis p<0.05*, p<0.005**.

FIG. 6 represents the same data differently. Each line represents a single experiment showing MFI of markers on the same Treg transduced with TCR or TCR+FOXP3.

### Example 4—Transduced Natural Tregs Compared to Induced Tregs

CD80⁺CD86⁺DR4⁺ CHO cells were loaded with peptide and irradiated as described in Example 10 before being re-suspended at 0.1×10⁶ cells/ml. Transduced responder T cells were stained with CFSE cell trace dye in warmed PBS at 37° C. for 3 minutes before addition of equal volumes of warm FBS and a further 3 minute incubation.

Cells were washed in 5× volume of complete media before being counted and resuspended at 1×10⁶ transduced cells/ml. The transduction efficiency of Tconv and Treg were determined by flow cytometry. Tregs were removed from culture, washed and resuspended at 1×10⁶ transduced cells/ml in complete RPMI. Cells were plated 1 Treg: 0.1 CHO cells: varying parts Tconv. Proliferation was determined by analysing dilution of carboxyfluorescein succinimidyl ester (CFSE)-stained Tconv.

The data in FIG. 7 show that TCR+FOXP3-transduced natural Tregs suppress proliferation more effectively than TCR+FOXP3-transduced Tconv cells (i.e. induced Tregs).

Supernatants were collected from the culture media and were assayed for IL-2 by ELISA. The data presented in FIG. 8 show that TCR+FOXP3-transduced natural Tregs suppress IL-2 production more effectively than TCR+FOXP3-transduced Tconv cells (i.e. induced Tregs).

### Example 5A—Treg Expressing Exogenous FOXP3 Engraft, Persist and Retain FoxP3, CD25 and TCR Expression

Thy1.1+CD4+CD25+ or CD45.1+CD4+CD25+ Treg were isolated from lymph nodes and splenocytes of HLA-DRB*0401 transgenic mice by bead sort. CD45.1+ Treg were transduced with TCR and Thy1.1+ Treg were transduced with TCR+murine FOXP3.1 day after transduction TCR or TCR+FOXP3 transduced cells were injected in a 1:1 ratio into HLA-DRB*0401 transgenic hosts conditioned with 4Gy irradiation. FACS plots show the ratio of CD45.1:Thy1.1 of injected cells and their respective FOXP3 expression.

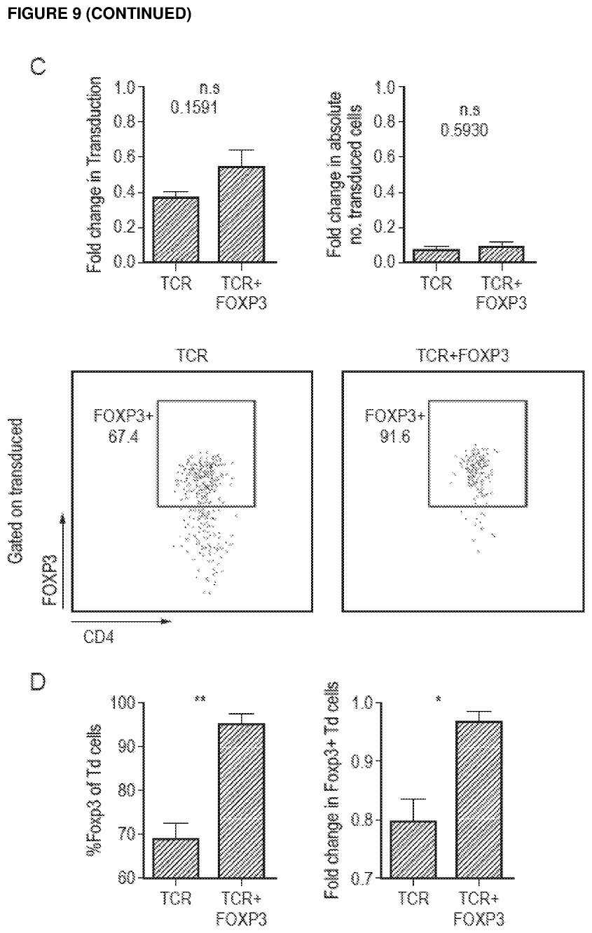

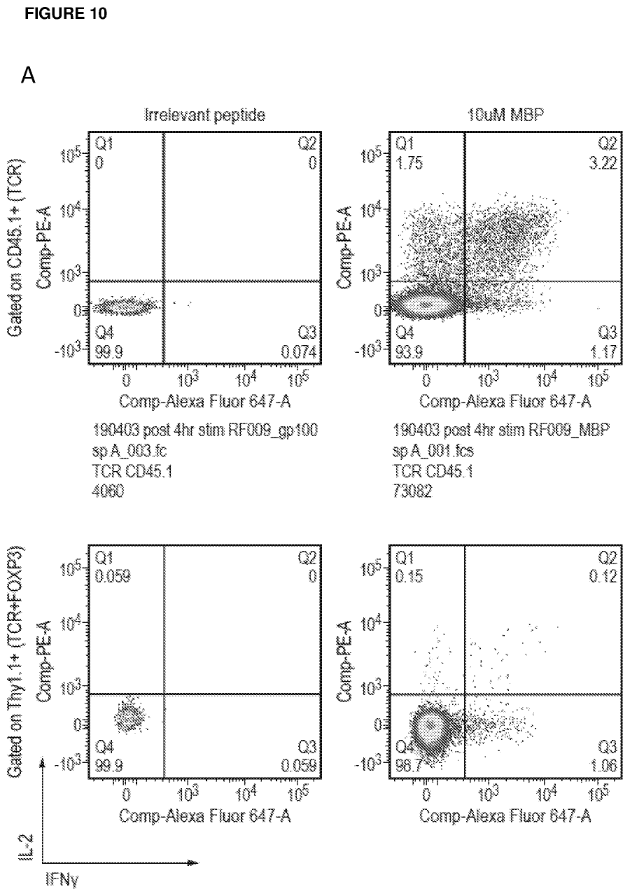

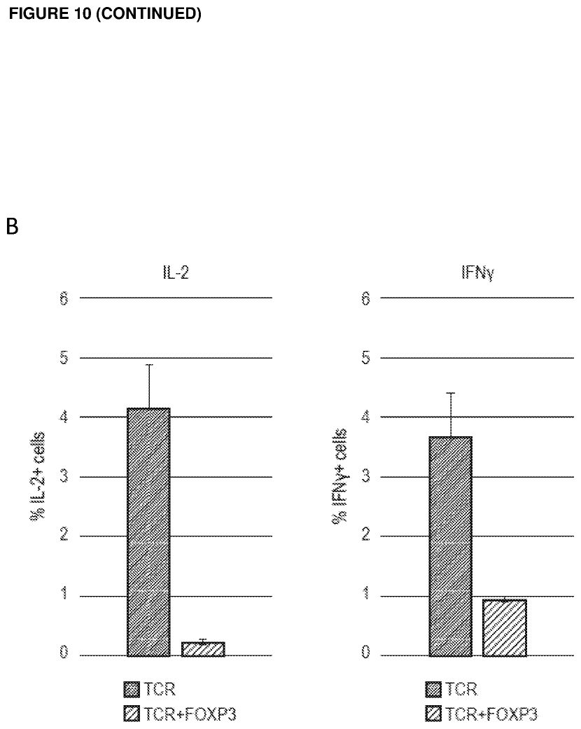

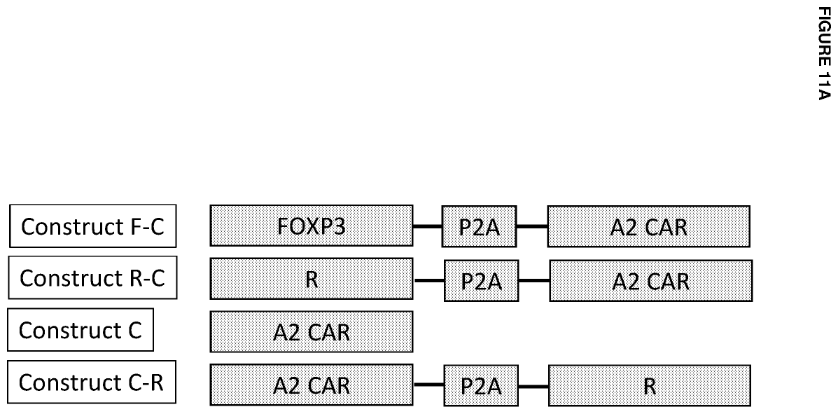

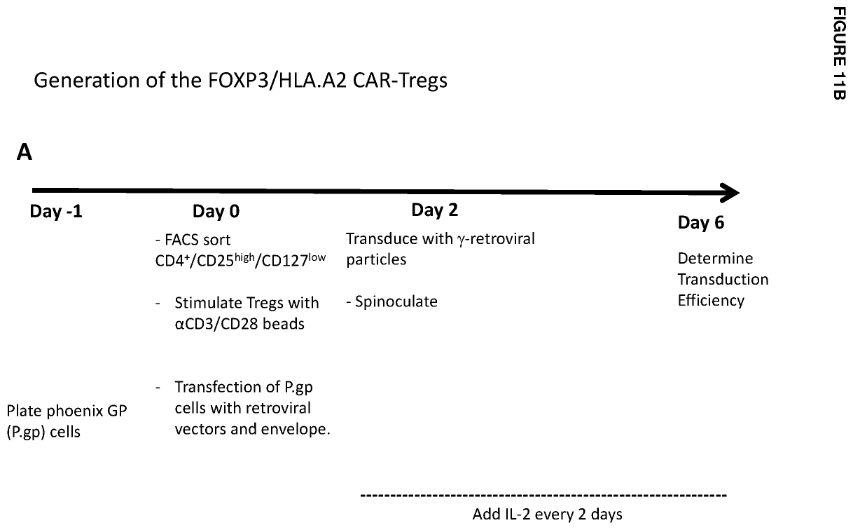

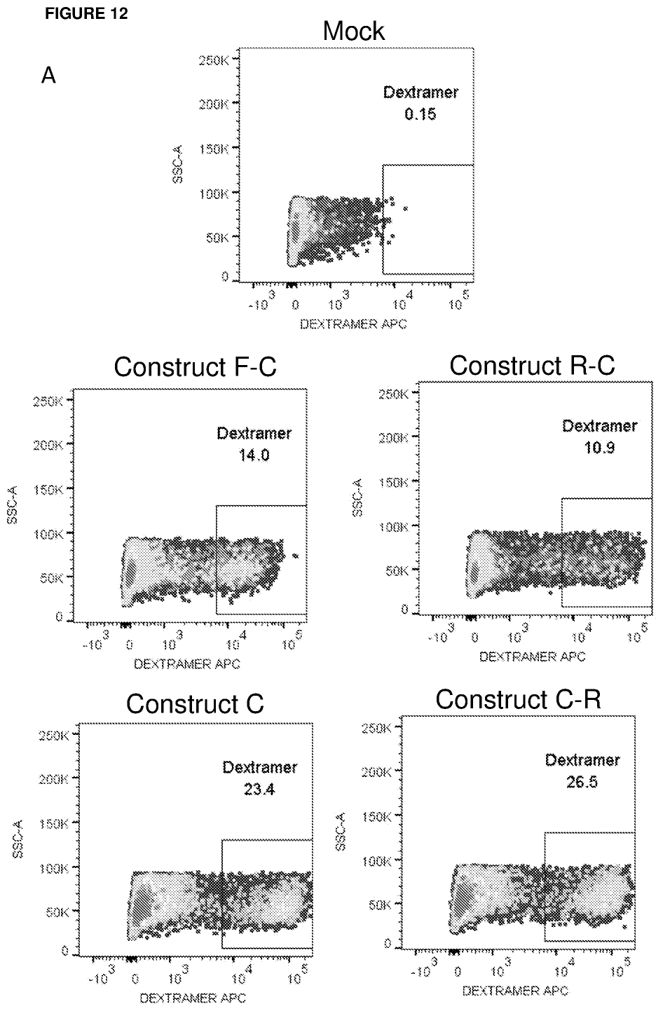

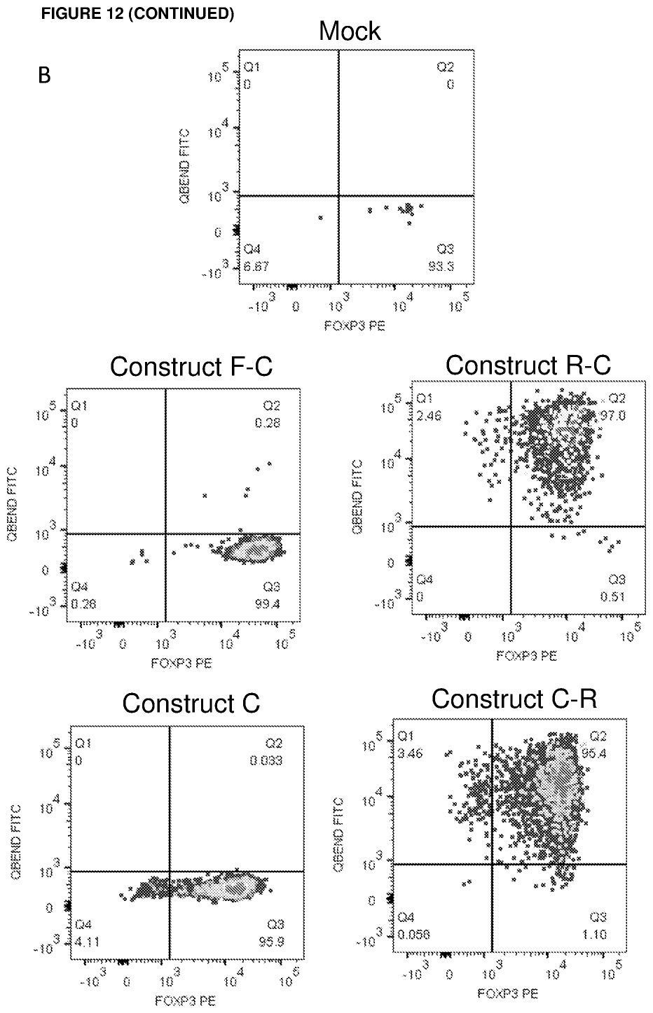

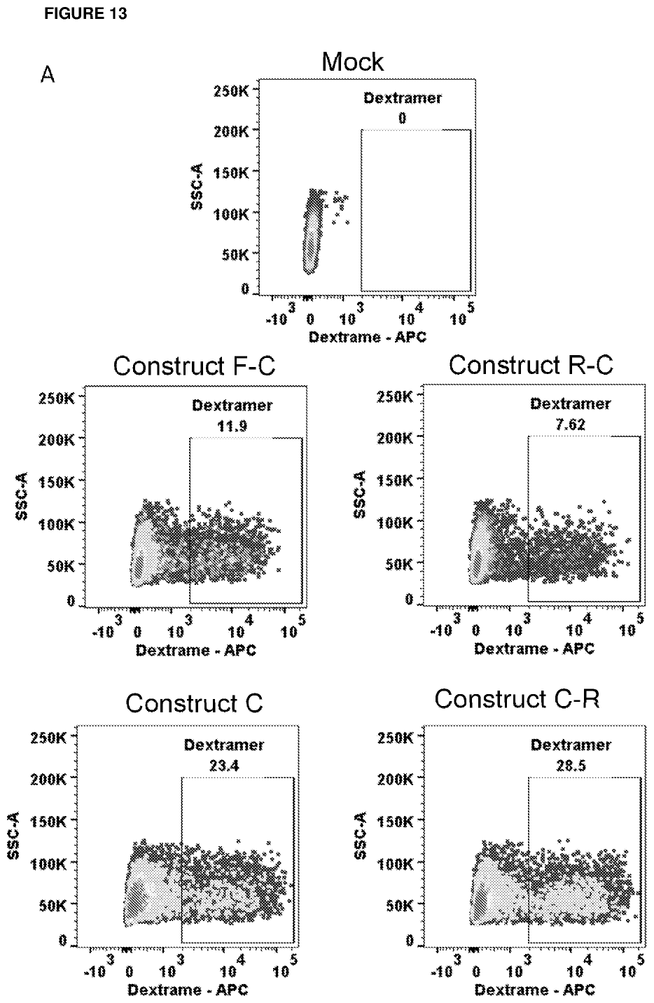

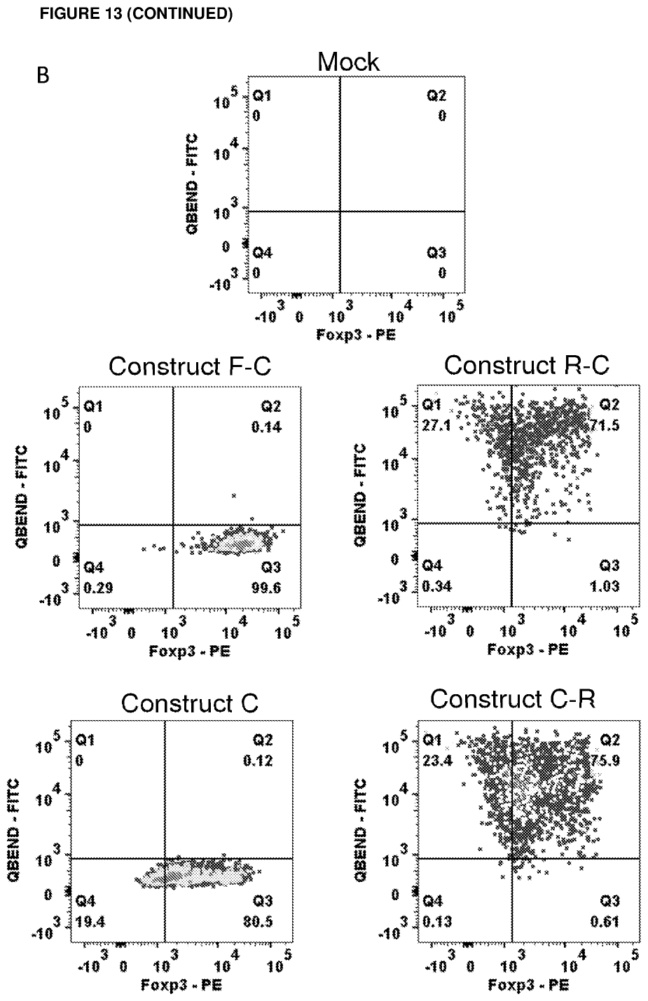

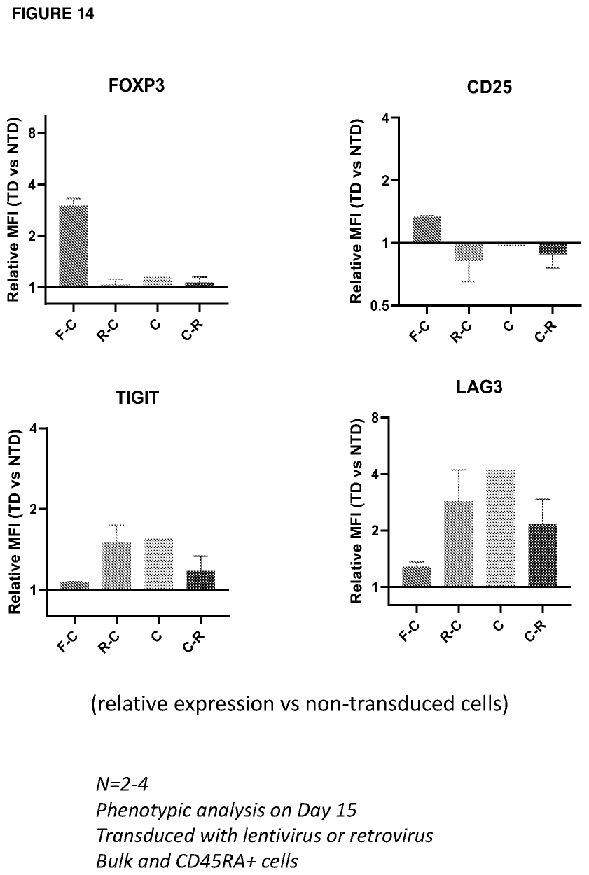

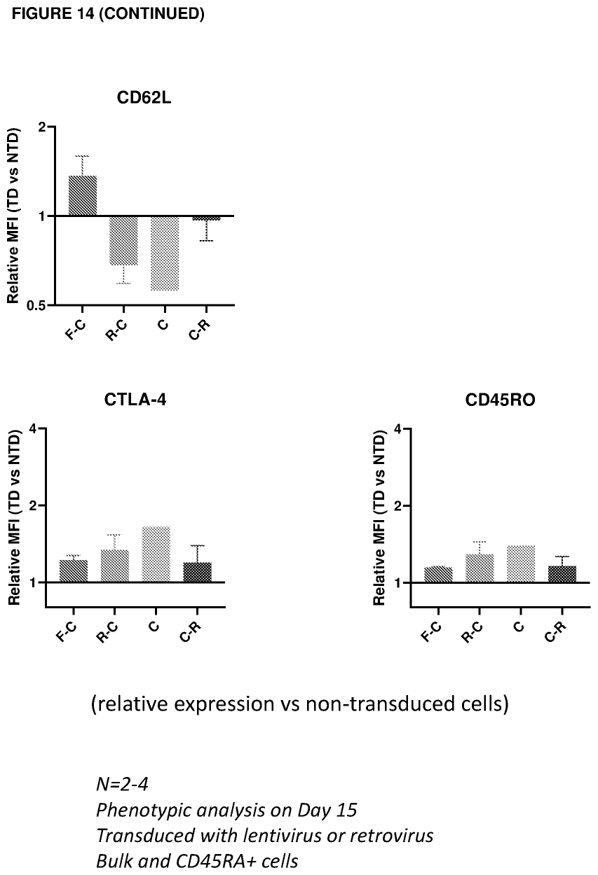

After 7 weeks flow cytometry was used to identify engrafted cells by staining for TCR. The ratio of CD45.1:Thy1.1 within the TCR+ population was determined and the phenotype of engrafted CD45.1 (Treg transduced with TCR) or Thy1.1 (Treg transduced with TCR+FOXP3) cells was examined by staining for FOXP3 and CD25. Thy1.1+CD4+CD25+ Treg were isolated from lymph nodes and splenocytes of HLA-DRB*0401 transgenic mice by bead sort. Treg were transduced TCR, TCR+murine FOXP3 or cultured with virus-free supernatant (mock). 1 day after transduction TCR or TCR+FOXP3 transduced cells were injected into HLA-DRB*0401 transgenic hosts conditioned with 4Gy irradiation. 7 weeks later flow cytometry was used to determine the engraftment of transduced Treg FIG. 9, A shows the transduction efficiency determined through expression of human variable 2.1 and murine Foxp3 on dl post-transduction. FIG. 9, B shows splenocytes from mice that received Treg transduced with TCR or TCR+FOXP3 stained with Thy1.1 to identify transferred cells (top panel) and FOXP3 and TCR (bottom panel). FIG. 9, C shows cumulative data showing fold change in transduction efficiency (left panel) and fold change in absolute number of transduced cells (right panel) relative to day of injection for Treg transduced with TCR or TCR+FOXP3. FIG. 9, D shows a representative expression of FOXP3 within transduced cells 7 weeks after transfer. Graphs show cumulative of percentage FOXP3+ cells within the transduced population at week 7 (left) and the fold change in FOXP3+ cells relative to the day of injection.

### Example 5B—Treg Expressing Exogenous FOXP3 Retain Treg Functionality after 7 Weeks In Vivo Whilst Tregs not Expressing Exogenous FOXP3 Acquire the Ability to Produce Effector Cytokines

Splenocytes were cultured for 4 hours with CD86+HLA-DR4+CHO cells pulsed with irrelevant peptide or 10 uM MBP. Treg expressing exogenous FOXP3 retain Treg functionality after 7 weeks in vivo as demonstrated by lack of effector cytokine production, whilst Tregs not expressing exogenous FOXP3 acquire the ability to produce effector cytokines (FIG. 10).

Although Examples 1-5 were exemplified using a TCR, the results are broadly applicable, for example to other TCR constructs or CAR constructs, e.g. comprising an antigen-binding domain which targets HLA-A2.

### Example 6A—Tregs Transduced with a Construct Encoding FOXP3 and an HLA-A2 Specific CAR Express Both Genes and Express FOXP3 at Noticeably Higher Levels than Tregs with Endogenous FOXP3 Only

Tregs were isolated from PBMCs by CD4+ and CD25+ enrichment. The enriched cells were stained for CD4, CD25, CD127 and CD45RA and sorted by FACS.

Enriched Tregs were transduced with one of four constructs. FIG. 11A shows a schematic diagram of the constructs used: Construct F-C: illustrates a construct encoding 5′-FOXP3-P2A-A2 CAR-3′; Construct R-C: illustrates a construct encoding 5′-R-P2A-A2 CAR-3′, where R is another gene; Construct C: illustrates a construct encoding the A2 CAR only; Construct C-R: illustrates a construct encoding 5′-A2 CAR-P2A-R-3′, where R is another gene.

FIG. 11B shows a schematic of the transduction method.

The enriched and transduced cells were expanded using the following protocol:
Day 0, 0.25×10⁶ per ml in TexMACS™ with Gibco™ Dynabeads™ Human T-Activator CD3/CD28
Day 2, 0.1×10⁶ per ml in TexMACS™ on retronectin coated plates with IL-2 and virus
Day 4-9, resuspension to 0.25×10⁶ per ml in TexMACS™ with IL-2 in T25 flasks/6 well plates
Day 15, resuspension to 0.25×10⁶ per ml in TexMACS™ with IL-2 in T25 flasks

FIG. 12 shows the HLA-A2-specific CAR (A2 CAR) expression levels, FOXP3 expression levels and the expression levels of another gene, R, in Tregs transduced with Construct F-C, Construct R-C, Construct C, and Construct C-R, compared to mock control Tregs, as determined by flow cytometry.

FIG. 12A shows that Tregs transduced with each construct expressed the HLA-A2-specific CAR. The HLA-A2-specific CAR was expressed from both constructs in which the CAR was downstream (Construct R-C) and when the CAR was upstream (Construct C-R).

FIG. 12B shows that FOXP3 was expressed in all Tregs, but was noticeably higher in Construct F-C, particularly when compared to expression of the HLA-A2-specific CAR alone (Construct C).

### Example 6B—Tregs Transduced with a Construct Encoding FOXP3 and an HLA-A2 Specific CAR Retain FOXP3 Expression

FIG. 13 shows the HLA-A2-specific CAR (A2 CAR) expression levels, FOXP3 expression levels and the expression levels of another gene, R, in extended expanded Tregs transduced with Construct F-C, Construct R-C, Construct C, and Construct C-R, compared to mock control Tregs, as determined by flow cytometry.

FIG. 13A shows that Tregs transduced with each construct still expressed the HLA-A2-specific CAR after extended expansion.

FIG. 13B shows that FOXP3 expression decreased in Tregs transduced with Construct R-C, Construct C, and Construct C-R after extended expansion. In contrast, FOXP3 expression did not decrease in Tregs transduced Construct F-C after extended expansion. Consequently, FOXP3 expression was substantially higher in Construct F-C after extended expansion, particularly when compared to expression of the HLA-A2-specific CAR alone (Construct C).

### Example 7—Tregs Transduced with a Construct Encoding FOXP3 and an HLA-A2 Specific CAR Maintain Treg Phenotypic Lineage while Enhancing FOXP3 Expression

CD4⁺CD25⁺CD127⁻ Tregs were FACs sorted, activated, transduced and expanded as described in Example 6A, except that the media used in the expansion protocol was switched to X-VIVO-15™ with 5% AB serum (rather than TexMACS™) and the concentration of cells at day 0 was 0.2×10⁶ per ml (rather than 0.25×10⁶ per ml).

T cells were then removed from culture and stained for the HLA-A2 specific CAR and live cells using the dextramer and LIVE/DEAD™ Fixable Near-IR. Consequently, surface staining of the cells was performed in Brilliant stain buffer (BD) containing the following antibodies; anti-CD25 PE-Cy7, anti-CD62L PE-CF594, anti-TIGIT BV605, anti-CD45RO BUV395, and anti-CD223 BV711 for 20-30 minutes at 4° C. in the dark. Cells were then washed with FACS buffer and resuspended in fixation/permeabilization solution. The cells were incubated at 4° C. for 30 min in the dark. Permeabilised cells were then washed with 1×permeabilization buffer and resuspended in 50 μL of 1× permeabilization buffer containing anti-CTLA-4 BV421 and anti-Foxp3 PE for 30 min in the dark at 4° C. Cells were then washed with 1× permeabilization buffer, resuspended in FACS buffer and analysed by flow cytometry.

In each sample, transduced (TD) cells were identified as dextramer+ and the remaining cells were considered non-transduced (NTD); mean fluorescence intensity (MFI) for each phenotypic lineage marker was determined in the TD and NTD cells. 2⁰ represents no change, 2¹ represents two-fold increased expression.

FIG. 14 shows phenotypic lineage marker expression in Tregs transduced with Construct F-C, Construct R-C, Construct C, and Construct C-R. Tregs transduced with construct F-C maintain Treg phenotypic lineage while enhancing FOXP3 expression.

All publications mentioned in the above specification are herein incorporated by reference. Various modifications and variations of the disclosed methods, cells, compositions and uses of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

1-51. (canceled)

51. A vector comprising a first polynucleotide encoding a FOXP3 polypeptide and a second polynucleotide encoding a chimeric antigen receptor (CAR), wherein the CAR comprises an antigen recognition domain which specifically binds to a human leukocyte antigen (HLA), wherein the first polynucleotide and the second polynucleotide are operably linked to the same promoter, and wherein the first polynucleotide is upstream of the second polynucleotide.

52. The vector according to claim 51, wherein the antigen recognition domain specifically binds to HLA-A2.

53. The vector according to claim 51, wherein:
(a) the vector comprises a polynucleotide encoding a cleavage site between the first polynucleotide and the second polynucleotide and/or an internal ribosome entry site (IRES) between the first polynucleotide and the second polynucleotide; and/or
(b) the vector comprises a self-cleaving sequence between the first polynucleotide and the second polynucleotide, preferably wherein the self-cleaving sequence is a polynucleotide sequence encoding a 2A self-cleaving peptide, optionally wherein the 2A self-cleaving peptide is selected from the group consisting of: P2A peptide, T2A peptide, E2A peptide, and F2A peptide; and/or
(c) the vector is a viral vector, preferably a retroviral vector or a lentiviral vector; and/or
(d) the promoter is a long terminal repeat (LTR).

54. The vector according to claim 51, wherein:
(a) the FOXP3 polypeptide comprises or consists of an amino acid sequence which has at least 70%, 80%, 85%, 90%, 95%, 98%, 99% or 100% identity to SEQ ID NOs: 1 or 2, or a functional fragment thereof; and/or
(b) the first polynucleotide comprises or consists of a polynucleotide sequence which has at least 70%, 80%, 85%, 90%, 95%, 98%, 99% or 100% identity to SEQ ID NOs: 3 or 4, or a functional fragment thereof; and/or
(c) the second polynucleotide comprises a polynucleotide sequence which has at least 70%, 80%, 85%, 90%, 95%, 98%, 99% or 100% identity to SEQ ID NO: 211 or SEQ ID NO: 212.

55. The vector according to claim 51, wherein the antigen recognition domain:
(a) is an antibody, an antibody fragment, or derived from an antibody; and/or
(b) is an antigen-binding fragment (Fab), a single chain antibody (scFv), or a single-domain antibody (sdAb); and/or
(c) comprises one or more CDR regions selected from SEQ ID NOs: 5-133 or derivatives thereof comprising one, two, or three amino acid substitutions; and/or
(d) comprises CDR1, CDR2 and CDR3 regions comprising or consisting of: SEQ ID NOs: 5-7, respectively; SEQ ID NOs: 8-10, respectively; SEQ ID NOs: 11-13, respectively; SEQ ID NOs: 14-16, respectively; SEQ ID NOs: 17-19, respectively; SEQ ID NOs: 20-22, respectively; SEQ ID NOs: 23-25, respectively; SEQ ID NOs: 26-28, respectively; SEQ ID NOs: 29-31, respectively; SEQ ID NOs: 32-34, respectively; SEQ ID NOs: 35-37, respectively; SEQ ID NOs: 38-40, respectively; SEQ ID NOs: 41-43, respectively; SEQ ID NOs: 44-46, respectively; SEQ ID NOs: 47-49, respectively; SEQ ID NOs: 50-52, respectively; SEQ ID NOs: 53-55, respectively; SEQ ID NOs: 56-58, respectively; SEQ ID NOs: 59-61, respectively; SEQ ID NOs: 62-64, respectively; SEQ ID NOs: 65-67, respectively; SEQ ID NOs: 68-70, respectively; SEQ ID NOs: 71-73, respectively; SEQ ID NOs: 74-76, respectively; SEQ ID NOs: 77-79, respectively; SEQ ID NOs: 80-82, respectively; SEQ ID NOs: 83-85, respectively; SEQ ID NOs: 86-88, respectively; SEQ ID NOs: 89-91, respectively; SEQ ID NOs: 92-94, respectively; SEQ ID NOs: 95-97, respectively; SEQ ID NOs: 98-100, respectively; SEQ ID NOs: 101-103, respectively; SEQ ID NOs: 104-106, respectively; SEQ ID NOs: 107-109, respectively; SEQ ID NOs: 110-112, respectively; SEQ ID NOs: 113-115, respectively; SEQ ID NOs: 116-118, respectively; SEQ ID NOs: 119-121, respectively; SEQ ID NOs: 122-124, respectively; SEQ ID NOs: 125-127, respectively; SEQ ID NOs: 128-130, respectively; and/or SEQ ID NOs: 131-133, respectively.

56. The vector according to claim 51, wherein the antigen recognition domain:
(a) comprises:
(i) a variable heavy domain which comprises CDR1, CDR2 and CDR3 regions comprising or consisting of SEQ ID NOs: 5-7, respectively, and a variable light domain which comprises CDR1, CDR2 and CDR3 regions comprising or consisting of SEQ ID NOs: 17-19, respectively;
(ii) a variable heavy domain which comprises CDR1, CDR2 and CDR3 regions comprising or consisting of SEQ ID NOs: 8-10, respectively, and a variable light domain which comprises CDR1, CDR2 and CDR3 regions comprising or consisting of SEQ ID NOs: 20-22, respectively;
(iii) a variable heavy domain which comprises CDR1, CDR2 and CDR3 regions comprising or consisting of SEQ ID NOs: 11-13, respectively, and a variable light domain which comprises CDR1, CDR2 and CDR3 regions comprising or consisting of SEQ ID NOs: 23-25, respectively;
(iv) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 26-28, respectively;
(v) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 29-31, respectively;
(vi) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 32-34, respectively;
(vii) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 35-37, respectively;
(viii) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 38-40, respectively;
(ix) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 41-43, respectively;
(x) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 44-46, respectively;
(xi) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 47-49, respectively;
(xii) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 50-52, respectively;
(xiii) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 53-55, respectively;
(xiv) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 56-58, respectively;
(xv) a variable heavy domain which comprises CDR1, CDR2 and CDR3 regions comprising or consisting of SEQ ID NOs: 14-16, respectively, and a variable light domain which comprises CDR1, CDR2 and CDR3 regions comprising or consisting of SEQ ID NOs: 59-61, respectively;
(xvi) a variable heavy domain which comprises CDR1, CDR2 and CDR3 regions comprising or consisting of SEQ ID NOs: 62-64, respectively, and a variable light domain which comprises CDR1, CDR2 and CDR3 regions comprising or consisting of SEQ ID NOs: 98-100, respectively;
(xvii) a variable heavy domain which comprises CDR1, CDR2 and CDR3 regions comprising or consisting of SEQ ID NOs: 65-67, respectively, and a variable light domain which comprises CDR1, CDR2 and CDR3 regions comprising or consisting of SEQ ID NOs: 101-103, respectively;
(xviii) a variable heavy domain which comprises CDR1, CDR2 and CDR3 regions comprising or consisting of SEQ ID NOs: 68-70, respectively, and a variable light domain which comprises CDR1, CDR2 and CDR3 regions comprising or consisting of SEQ ID NOs: 104-106, respectively;
(xix) a variable heavy domain which comprises CDR1, CDR2 and CDR3 regions comprising or consisting of SEQ ID NOs: 71-73, respectively, and a variable light domain which comprises CDR1, CDR2 and CDR3 regions comprising or consisting of SEQ ID NOs: 107-109, respectively;
(xx) a variable heavy domain which comprises CDR1, CDR2 and CDR3 regions comprising or consisting of SEQ ID NOs: 74-76, respectively, and a variable light domain which comprises CDR1, CDR2 and CDR3 regions comprising or consisting of SEQ ID NOs: 110-112, respectively;
(xxi) a variable heavy domain which comprises CDR1, CDR2 and CDR3 regions comprising or consisting of SEQ ID NOs: 77-79, respectively, and a variable light domain which comprises CDR1, CDR2 and CDR3 regions comprising or consisting of SEQ ID NOs: 113-115, respectively;
(xxii) a variable heavy domain which comprises CDR1, CDR2 and CDR3 regions comprising or consisting of SEQ ID NOs: 80-82, respectively, and a variable light domain which comprises CDR1, CDR2 and CDR3 regions comprising or consisting of SEQ ID NOs: 116-118, respectively;
(xxiii) a variable heavy domain which comprises CDR1, CDR2 and CDR3 regions comprising or consisting of SEQ ID NOs: 83-85, respectively, and a variable light domain which comprises CDR1, CDR2 and CDR3 regions comprising or consisting of SEQ ID NOs: 119-121, respectively;
(xxiv) a variable heavy domain which comprises CDR1, CDR2 and CDR3 regions comprising or consisting of SEQ ID NOs: 86-88, respectively, and a variable light domain which comprises CDR1, CDR2 and CDR3 regions comprising or consisting of SEQ ID NOs: 122-124, respectively;
(xxv) a variable heavy domain which comprises CDR1, CDR2 and CDR3 regions comprising or consisting of SEQ ID NOs: 89-91, respectively, and a variable light domain which comprises CDR1, CDR2 and CDR3 regions comprising or consisting of SEQ ID NOs: 125-127, respectively;
(xxvi) a variable heavy domain which comprises CDR1, CDR2 and CDR3 regions comprising or consisting of SEQ ID NOs: 92-94, respectively, and a variable light domain which comprises CDR1, CDR2 and CDR3 regions comprising or consisting of SEQ ID NOs: 128-130, respectively; or
(xxvii) a variable heavy domain which comprises CDR1, CDR2 and CDR3 regions comprising or consisting of SEQ ID NOs: 95-97, respectively, and a variable light domain which comprises CDR1, CDR2 and CDR3 regions comprising or consisting of SEQ ID NOs: 131-133, respectively; and/or
(b) comprises a variable heavy domain which has at least about 80%, 85%, 90%, 95%, 98%, 99%, or 100% identity to one or more of SEQ ID NOs: 134-149 and/or comprises a variable light domain which has at least about 80%, 85%, 90%, 95%, 98%, 99%, or 100% identity to one or more of SEQ ID NOs: 150-176; and/or
(c) comprises or consists of an amino acid sequence which has at least 80%, 85%, 90%, 95%, 98%, 99% or 100% identity to one or more of SEQ ID NOs: 177-203.

57. The vector according to claim 51, wherein:
(a) the CAR comprises a transmembrane (TM) domain and an intracellular signalling domain, and optionally wherein the CAR comprises a hinge domain and/or one or more co-stimulatory domains; and/or
(b) the CAR comprises one or more hinge domains selected from the group consisting of a CD28 hinge domain, a CD8 hinge domain, an IgG hinge domain, and an IgD hinge domain, preferably wherein the CAR comprises a CD8 hinge domain; and/or
(c) the CAR comprises one or more TM domains selected from the group consisting of a CD28 TM domain, an ICOS TM domain, a CD8 TM domain, a CD4 TM domain, an OX40 TM domain, a 4-1BB TM domain, and a CD3 zeta TM domain, preferably wherein the CAR comprises a CD8 TM domain; and/or
(d) the CAR comprises one or more co-stimulatory domains selected from the group consisting of a CD28 signalling domain, an ICOS signalling domain, an OX40 signalling domain, a 4-1BB signalling domain, a CD27 signalling domain, or a TNFRSF25 signalling domain, preferably wherein the CAR comprises a CD28 signalling domain; and/or
(e) the CAR comprises one or more intracellular signalling domains selected from the group consisting of a CD3 zeta signalling domain or any of its homologs, a CD3 polypeptide, a syk family tyrosine kinase, a src family tyrosine kinase, a CD2 signalling domain, a CD5 signalling domain and a CD8 signalling domain, preferably wherein the CAR comprises a CD3 zeta signalling domain; and/or
(f) the CAR comprises: a CD8 hinge domain; a CD8 TM domain; a CD28 signalling domain; and a CD3 zeta signalling domain; and/or
(g) the CAR comprises an amino acid sequence which has at least 80%, 85%, 90%, 95%, 98%, 99% or 100% identity to SEQ ID NO: 209 or SEQ ID NO: 210.

58. The vector according to claim 51, wherein the vector comprises:
(i) a first polynucleotide which comprises or consists of a polynucleotide sequence which has at least 70% identity to SEQ ID NO: 3, or a functional fragment thereof, optionally a self-cleaving sequence which has at least 70% identity to SEQ ID NO: 214, and a second polynucleotide which comprises a polynucleotide sequence which has at least 70% identity to SEQ ID NO: 211;
(ii) a first polynucleotide which comprises or consists of a polynucleotide sequence which has at least 70% identity to SEQ ID NO: 3, or a functional fragment thereof, optionally a self-cleaving sequence which has at least 70% identity to SEQ ID NO: 214, and a second polynucleotide which comprises a polynucleotide sequence which has at least 70% identity to SEQ ID NO: 212;
(iii) a first polynucleotide which comprises or consists of a polynucleotide sequence which has at least 70% identity to SEQ ID NO: 4, or a functional fragment thereof, optionally a self-cleaving sequence which has at least 70% identity to SEQ ID NO: 214, and a second polynucleotide which comprises a polynucleotide sequence which has at least 70% identity to SEQ ID NO: 211; or
(iv) a first polynucleotide which comprises or consists of a polynucleotide sequence which has at least 70% identity to SEQ ID NO: 4, or a functional fragment thereof, optionally a self-cleaving sequence which has at least 70% identity to SEQ ID NO: 214, and a second polynucleotide which comprises a polynucleotide sequence which has at least 70% identity to SEQ ID NO: 212.

59. An engineered T cell comprising a vector according to claim 51.

60. The engineered T cell according to claim 59, wherein the engineered T cell is an engineered regulatory T cell (Treg).

61. A method for enhancing the ability of an engineered HLA-specific Treg to suppress an immune response which comprises the step of introducing into the Treg a polynucleotide encoding a FOXP3 polypeptide or a vector according to claim 51, wherein the immune response is preferably an immune response against a cell expressing the HLA, preferably wherein the HLA is HLA-A2.

62. A method for enhancing the ability of an engineered HLA-specific Treg to suppress an immune response comprising introducing a first polynucleotide encoding a FOXP3 polypeptide and a second polynucleotide encoding a HLA-specific CAR into:
(a) the Treg or
(b) a cell-containing sample, wherein:
(i) the cell-containing sample comprises or consists of Tregs; and/or
(ii) the cell-containing sample comprises or consists of peripheral blood mononuclear cells (PBMCs) and Tregs are enriched from the cell-containing sample prior to or after introducing the first polynucleotide and/or second polynucleotide; and/or
(iii) the cell-containing sample comprises or consists of PBMCs and Tregs are generated from the cell-containing sample prior to or after introducing the first polynucleotide and/or the second polynucleotide,
preferably wherein the HLA is HLA-A2.

63. The method according to claim 62, wherein:
(a) the first polynucleotide and/or the second polynucleotide are introduced by viral transduction, preferably retroviral or lentiviral transduction; and/or
(b) the first polynucleotide and the second polynucleotide are introduced in a single vector, optionally wherein the first polynucleotide and the second polynucleotide are operably linked to the same promoter; and/or
(c) the method comprises introducing a vector comprising the first and second polynucleotides, wherein the first polynucleotide and the second polynucleotide are operably linked to the same promoter, and wherein the first polynucleotide is upstream of the second polynucleotide.

64. A method of reducing the risk of (i) an engineered HLA-specific Treg acquiring an effector phenotype and/or (ii) generating an engineered HLA-specific T effector cell during generation of engineered HLA-specific Tregs, which method comprises a step of introducing into the Treg a polynucleotide encoding a FOXP3 polypeptide or a vector according to claim 51, preferably wherein the HLA is HLA-A2.

65. A method for reducing the risk of (i) an engineered HLA-specific Treg acquiring an effector phenotype; and/or (ii) generating an engineered HLA-specific T effector cell during generation of engineered HLA-specific Tregs, comprising introducing a vector according to claim into a cell-containing sample, wherein:
(a) the cell-containing sample comprises or consists of Tregs and/or effector cells; and/or
(b) the cell-containing sample comprises or consists of PBMCs and Tregs are enriched from the cell-containing sample prior to or after introducing the vector; and/or
(c) the cell-containing sample comprises or consists of PBMCs and Tregs are generated from the cell-containing sample prior to or after introducing the vector, preferably wherein the HLA is HLA-A2.

66. An engineered Treg obtainable or obtained by a method according to claim 62.

67. A pharmaceutical composition comprising an engineered Treg according to claim 66.

68. A method of inducing tolerance to a transplant in a subject, or treating and/or preventing of transplant rejection or GvHD in a subject, comprising administering an engineered Treg according to claim 66 to the subject.

69. The method of claim 68, wherein the method comprises the following steps:
(i) isolation or provision of a cell-containing sample from the subject, wherein the cell-containing sample comprises or consists of PBMCs; and
(ii) introducing a vector into the cell-containing sample, wherein the vector comprises a first polynucleotide encoding a FOXP3 polypeptide and a second polynucleotide encoding a chimeric antigen receptor (CAR), wherein the CAR comprises an antigen recognition domain which specifically binds to a human leukocyte antigen (HLA), wherein the first polynucleotide and the second polynucleotide are operably linked to the same promoter, and wherein the first polynucleotide is upstream of the second polynucleotide;
wherein the cell-containing sample comprises or consists of Tregs; and/or wherein Tregs are enriched from the cell-containing sample prior to or after introducing the vector; and/or wherein Tregs are generated from the cell-containing sample prior to or after introducing the vector, and optionally wherein the subject is human.
